SEQUENCE LISTING (11 sequences)

SEQ ID NO: 1 - protein, 5 aa, synthetic construct
GYGMH

SEQ ID NO: 2 - skipped

SEQ ID NO: 3 - protein, 17 aa, synthetic construct
VIWYDGSKKYYVDSVKG

SEQ ID NO: 4 - protein, 9 aa, synthetic construct
QMGYWHFDL

SEQ ID NO: 5 - protein, 11 aa, synthetic construct
RASQSVSSYLA

SEQ ID NO: 6 - protein, 7 aa, synthetic construct
DASNRAT

SEQ ID NO: 7 - protein, 10 aa, synthetic construct
QQRSNWPPLT

SEQ ID NO: 8 - protein, 118 aa, synthetic construct [sequence omitted]

SEQ ID NO: 9 - protein, 108 aa, synthetic construct [sequence omitted]

SEQ ID NO: 10 - protein, 448 aa, synthetic construct
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 215 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0176359 A1
(43) Pub. Date: Jun. 25, 2026

(54) METHODS OF SUPPRESSING MICROGLIAL ACTIVATION

(71) Applicants: The Brigham and Women's Hospital, Inc., Boston, MA (US); Tiziana Life Sciences PLC, London (GB)

(72) Inventors: Howard L. WEINER, Brookline, MA (US); Tanuja CHITNIS, Chestnut Hill, MA (US); Tarun SINGHAL, Cambridge, MA (US); Kunwar SHAILUBHAI, Line Lexington, PA (US)

(21) Appl. No.: 19/534,143

(22) Filed: Feb. 9, 2026

Related U.S. Application Data
(63) Continuation of application No. 18/700,972.
(60) Provisional application No. 63/255,809, filed on Oct. 14, 2021.
(60) Provisional application No. 63/315,331, filed on Mar. 1, 2022.
(60) Provisional application No. 63/349,422, filed on Jun. 6, 2022.

(51) Int. Cl. C07K 16/28; A61K 9/00; A61K 39/00; A61P 9/10; A61P 25/28

(52) U.S. Cl. CPC C07K 16/2809; A61K 9/007; A61P 9/10; A61P 25/28; A61K 2039/505; C07K 2317/21; C07K 2317/565; C07K 2317/71

(57) ABSTRACT

The present disclosure provides methods of suppressing the activation of microglial cells, methods of ameliorating or treating the neurological effects of cerebral ischemia or cerebral inflammation, and methods of ameliorating or treating specific diseases that affect the CNS by nasally administering an anti-CD3 antibody.

### CROSS-REFERENCE TO RELATED APPLICATIONS

The present application is a continuation of U.S. patent application Ser. No. 18/700,972, filed Apr. 12, 2024, which is a U.S. National Phase application, filed under 35 U.S.C. § 371, of International Application No. PCT/US2022/078174, which claims the benefit of U.S. Provisional Patent Application No. 63/255,809 filed Oct. 14, 2021, U.S. Provisional Patent Application No. 63/315,331, filed Mar. 1, 2022, and U.S. Provisional Patent Application No. 63/349,422, filed Jun. 6, 2022, each of which is incorporated herein by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “TIZI-034_C03US_SeqList_ST26.xml”. The XML file is 10,764 bytes, created on Feb. 9, 2026, and is being submitted electronically via USPTO Patent Center.

### FIELD OF THE INVENTION

The present invention relates to generally to methods of ameliorating or treating the neurological effects of microglial activation and methods of ameliorating or treating specific diseases that affect the CNS by administering an anti-CD3 antibody.

### BACKGROUND OF THE INVENTION

Human CD3 antigen consists of a minimum of four invariant polypeptide chains, which are non-covalently associated with the T-cell receptors on the surface of T-cells, and is generally now referred to as the CD3 antigen complex. It is intimately involved in the process of T-cell activation in response to antigen recognition by the T-cell receptors.

Due to the fundamental nature of CD3 in initiating an anti-antigen response, monoclonal antibodies against this receptor have been proposed as being capable of blocking or at least modulating the immune process and thus as agents for the treatment of inflammatory and/or autoimmune disease.

The Central Nervous System (CNS) has long been considered to be a site of relative immune privilege. However, it is increasingly recognized that CNS tissue injury in acute and chronic neurological disease may be mediated by the CNS inflammatory response. The CNS inflammatory response is primarily mediated by inflammatory cytokines.

There is a need in the art for a more specific therapeutic targeting system to control microglial activation and neuroinflammation.

### SUMMARY OF THE INVENTION

In one aspect, provided herein is a method of treating or alleviating a sign or symptom of a disease associated with microglial activation in a subject, comprising intra-nasally administering to a subject a daily dose of about 10 μg-200 μg of an anti-CD3 antibody. In some embodiments, the disease associated with microglial activation is a neurodegenerative disorder, an ischemic related disease or injury, traumatic brain injury or a lysosomal storage disease. In some embodiments, the neurodegenerative disease is Multiple Sclerosis (MS), Alzheimer's disease (AD), Lewy Body Disease, Parkinson's Disease (PD), Parkinson's Disease (PD) Huntington's Disease (HD), Amyotrophic Lateral Sclerosis (ALS), epilepsy, HIV-associated encephalopathy or AIDS related dementia. In some embodiments, the ischemic related disease is a ischemic-reperfusion injury, stroke, myocardial infarction. In some embodiments, the ischemic-reperfusion injury is in lung tissue, cardiac, tissue and neuronal tissue. In some embodiments, the traumatic brain injury is a concussion or whiplash. In some embodiments, the concussion is a repetitive concussive injury. In some embodiments, the lysosomal storage disease is Neimann-Pick disease. In some embodiments, the sign or symptom of a disease associated with microglial activation is amyloid plaque formation.

In some embodiments, the anti-CD3 antibody is a monoclonal or polyclonal antibody. In some embodiments, the anti-CD3 antibody is a fully human, humanized or chimeric. In some embodiments, the anti-CD3 antibody comprises a heavy chain complementarity determining region 1 (CDRH1) comprising the amino acid sequence GYGMH (SEQ ID NO: 1), a heavy chain complementarity determining region 2 (CDRH2) comprising the amino acid sequence VIWYDGSKKYYVDSVKG (SEQ ID NO: 3), a heavy chain complementarity determining region 3 (CDRH3) comprising the amino acid sequence QMGYWHFDL (SEQ ID NO: 4), a light chain complementarity determining region 1 (CDRL1) comprising the amino acid sequence RASQSVSSYLA (SEQ ID NO: 5), a light chain complementarity determining region 2 (CDRL2) comprising the amino acid sequence DASNRAT (SEQ ID NO: 6), and a light chain complementarity determining region 3 (CDRL3) comprising the amino acid sequence QQRSNWPPLT (SEQ ID NO: 7). In some embodiments, the anti-CD3 antibody comprises a variable heavy chain amino acid sequence comprising the amino acid sequence of SEQ ID NO: 8 and a variable light chain amino acid sequence comprising the amino acid sequence of SEQ ID NO: 9. In some embodiments, the anti-CD3 antibody comprises a heavy chain amino acid sequence comprising the amino acid sequence of SEQ ID NO: 10 and a light chain amino acid sequence comprising the amino acid sequence of SEQ ID NO: 11.

In some embodiments, the daily doses is administered once a day. In some embodiments, the daily dose is 50 μg. In some embodiments, the daily dose is split equally between each nostril. In some embodiments, the daily dose is administered three times a week. In some embodiments, the daily doses is administered to the subject in at least one cycle, where the cycle is once daily three times a week for two weeks. In some embodiments, the cycle is repeated 2 to 10 times. In some embodiments, the cycle is followed by a drug holiday. In some embodiments, the drug holiday is a week.

In some embodiments, the method results in an improvement in EDSS scores in the subject of at least 10%, at least 20%, at least 30%, at least 40%, or at least 50%, compared to the EDSS scores prior to the administration of the anti-CD3 antibody. In some embodiments, the method results in an improvement in pyramidal scores in the subject of at least 10%, at least 20%, at least 30%, at least 40%, or at least 50%, compared to the pyramidal scores prior to the administration of the anti-CD3 antibody. In some embodiments, the method results in an improvement in the ability to walk as measured by the 25-foot timed walk test in the subject of at least 2 seconds, at least 3 seconds, at least 5 seconds, at least 10 seconds, at least 15 seconds, or at least 20 seconds compared to the ability to walk prior to the administration of the anti-CD3 antibody. In some embodiments, the method results in a reduction in microglial activation as measured by PET scan in the subject of at least 10%, at least 20%, at least 30%, at least 40%, or at least 50%, compared to the levels of microglial activation prior to the administration of the anti-CD3 antibody. In some embodiments, the method results in a reduction in the levels of IL-6, IL-1B, IFN-γ, and/or IL-18 in the subject of at least 10%, at least 20%, at least 30%, at least 40%, or at least 50%, compared to the levels prior to the administration of the anti-CD3 antibody. In some embodiments, the method results in an increase in the levels of CD8 naïve cells and/or a decrease in CD8 effector cells in the subject of at least 10%, at least 20%, at least 30%, at least 40%, or at least 50%, compared to the levels prior to the administration of the anti-CD3 antibody.

In another aspect, provided herein is a method of treating or alleviating a sign or symptom of a disease associated with neural inflammation in a subject, comprising intra-nasally administering to a subject a daily dose of about 10 μg-200 μg of an anti-CD3 antibody. In some embodiments, the disease is Multiple Sclerosis (MS), Alzheimer's disease (AD), Lewy Body Disease, Parkinson's Disease (PD), Parkinson's Disease (PD), or Amyotrophic Lateral Sclerosis (ALS).

In some embodiments, the anti-CD3 antibody is a monoclonal or polyclonal antibody. In some embodiments, the anti-CD3 antibody is a fully human, humanized or chimeric. In some embodiments, the anti-CD3 antibody comprises a heavy chain complementarity determining region 1 (CDRH1) comprising the amino acid sequence GYGMH (SEQ ID NO: 1), a heavy chain complementarity determining region 2 (CDRH2) comprising the amino acid sequence VIWYDGSKKYYVDSVKG (SEQ ID NO: 3), a heavy chain complementarity determining region 3 (CDRH3) comprising the amino acid sequence QMGYWHFDL (SEQ ID NO: 4), a light chain complementarity determining region 1 (CDRL1) comprising the amino acid sequence RASQSVSSYLA (SEQ ID NO: 5), a light chain complementarity determining region 2 (CDRL2) comprising the amino acid sequence DASNRAT (SEQ ID NO: 6), and a light chain complementarity determining region 3 (CDRL3) comprising the amino acid sequence QQRSNWPPLT (SEQ ID NO: 7). In some embodiments, the anti-CD3 antibody comprises a variable heavy chain amino acid sequence comprising the amino acid sequence of SEQ ID NO: 8 and a variable light chain amino acid sequence comprising the amino acid sequence of SEQ ID NO: 9. In some embodiments, the anti-CD3 antibody comprises a heavy chain amino acid sequence comprising the amino acid sequence of SEQ ID NO: 10 and a light chain amino acid sequence comprising the amino acid sequence of SEQ ID NO: 11.

In some embodiments, the daily doses is administered once a day. In some embodiments, the daily dose is 50 μg. In some embodiments, the daily dose is split equally between each nostril. In some embodiments, the daily dose is administered three times a week. In some embodiments, the daily doses is administered to the subject in at least one cycle, where the cycle is once daily three times a week for two weeks. In some embodiments, the cycle is repeated 2 to 10 times. In some embodiments, the cycle is followed by a drug holiday. In some embodiments, the drug holiday is a week. In some embodiments, the method results in a reduction of neural inflammation in the subject of at least 10%, at least 20%, at least 30%, at least 40%, or at least 50%, compared to the levels of neural inflammation prior to the administration of the anti-CD3 antibody.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 shows pre- and post-Foralumab treatment PET images.

FIG. 2 shows PET images showing a marked reduction in [F-18]PBR06 uptake in a high-affinity binder SPMS subject after 3 months of nasal Foralumab treatment (Top row). In comparison, test-retest [F-18]PBR06-PET scans performed in another high-affinity binder secondary-progressive multiple sclerosis (SPMS) subject (without any therapeutic intervention in between the scans) did not show significant difference on visual interpretation (Bottom row)

FIG. 3 shows PET images showing sustained reduction in [F-18]PBR06 uptake in a high-affinity binder SPMS subject after a 7 week drug holiday following 3 months of nasal Foralumab treatment.

FIG. 4 shows bar charts shown standardized [F-18]PBR06 uptake values (SUV) in whole brain, cortex, thalamus, white matter and cerebellum at baseline, 3 months and at 4.7 months.

FIG. 5 shows bar charts shown standardized [F-18]PBR06 uptake value ratio (SUVR) in whole brain, cortex, thalamus, white matter and cerebellum at baseline, 3 months and at 4.7 months.

FIG. 6 shows charts showing SUV and SUVR in whole brain, cortex, thalamus, white matter and cerebellum at baseline, 3 months and at 4.7 months compared to a pseudo-reference region.

FIGS. 7A-7G. Foralumab stimulation of PBMCs in vitro. FIG. 7A shows a representation of FACS data showing frequency of T cells that are CD4+ or CD4− that have undergone cell trace dilution after a 5 day peripheral blood mononuclear cell (PBMC) stimulation with UCHT1 or Foralumab (1 μg/ml) and IL-2 (5 U/ml) with or without anti-CD28 (0.5 μg/ml). FIG. 7B shows bar plots demonstrating the percent of the expanded T cells from PBMC cultures stimulated with UCHT1 or Foralumab that express CD4 (data from three healthy donors). FIG. 7C shows bar plot comparison of the percent of T cells that proliferated (i.e., gave cell trace dilution) within CD4 vs. CD8 T cells in the indicated 5-day PBMC cultures. FIGS. 7D-7F show representative FACS plots showing proliferation (cell trace dilution) from Foralumab or UCHT1 stimulated cultures of CD4 T cells only (FIG. 7D) or CD4 and CD8 T cells (Pan T cells, FIG. 7E) derived from the same PBMCs. All were stimulated in the presence of irradiated T cell depleted PBMCs as APCs and IL-2 (5 U/ml).

FIG. 7F shows a demonstration of potential co-expression of IFNγ and IL-17 in CD4 T cells from a different healthy donor stimulated with the indicated anti-CD3 mAbs and IL-2 or IL-2/anti-CD28 is shown. Purified CD4 T cells or CD4 and CD8 T cells (derived by Pan T cell isolation of PBMCs) were stimulated with Foralumab or UCHT1 (1 μg/ml) and irradiated T-cell depleted PBMCs as APCs in the indicated presence or absence of anti-CD28 (0.5 μg/ml CD28.2 mAb, BD) and IL-2 (5 U/ml) (FIG. 7G). Significance by One Way ANOVA (with Sidak's correction for multiple comparisons, *p<0.05, **p<0.001).

FIGS. 8A-8D. Surface CD3 and Foralumab dosing Longitudinal PBMCs were stained for CD3 and measured for changes in frequency of CD3⁺ cells or the intensity of CD3 (MFI) as compared to baseline (T1) levels (FIG. 8A). FIG. 8B shows lineage and differentiation markers in 10 μg, 50 μg, 250 μg and placebo groups. CD8⁺ refers to CD8⁺CD45RA⁻CD27⁻CD8 Tem, CD4⁺CD45RA⁺CD27⁺ naïve CD4 T and, CD3⁺CD4⁻CD8⁻ DN. C, D. Representative dot plots of CD8 TEMRA, naïve CD8 T cells, and CD8 T cell GzmB expression in all cohorts. FIG. 8C shows changes in frequency of naïve CD8 T cells (CD45RA⁺CD27⁺) and CD8 TEMRA cells (CD45RA⁺CD27−) are shown. FIG. 8D shows changes in the frequency of ex vivo CD8 T cell expression of GzmB is shown. In each group, the change with time was estimated using a linear mixed effects model with a fixed categorical effect of time and a random intercept. *p<0.05,**p<0.001).

FIGS. 9A-9G. RNA-seq analysis on PBMCs from healthy volunteers treated with 50 μg of Foralumab. FIG. 9A is a graphical depiction of the single cell analysis of the CD8+ population isolated from PBMCs showing the cell types that were defined by the clusters based on unbiased DEG the changes in CD8 maturational state subsets derived from the scRNA data. FIG. 9B shows the data in aggregated bar graphs or line graph analyses. Different maturational subsets of CD8 T cells show unique DEG between baseline (T1) and T2. FIGS. 9C-9E are heatmap presentation of the genes that exhibited increased or decreased expression from baseline after Foralumab treatment. Gene expression values were used to separate the cells into naïve CD8 T cells, naïve-like CD8 T cells (differed from naïve in expression of top group of genes), intermediate CD8 T cells (exhibited features of both naïve and memory cells), effector memory, and TEMRA (FIG. 9C). Different maturational subsets of CD4 T cells Gene expression values were used to separate the cells into naïve CD4 T cells, intermediate CD4 T cells (exhibited features of both naïve and memory cells), memory CD4, and memory CD4 with strong GALS1 gene expression (FIG. 9D). Different functional subsets of monocytes. Gene expression values were used to separate the cells into classical, non-classical and intermediate subsets (FIG. 9E). FIG. 9F is a series of violin plots showing changes in expression of TIGIT, TGFb1 and KIR3DL2 in CD8 effector memory and CD8 TEMRA cells. FIG. 9G is a series of violin plots showing changes in expression of CTLA4, KLRG1, and TGFb1 in naïve CD4 T cells and memory CD4 T cells. Unpaired two-sided T-test against timepoint 1 (T1vT2, T1vT3, T1vT4) was used to get the posted significance score if it changed from the baseline. *p<0.05, **p<0.001, ***p<0.0001).

FIGS. 10A and B. Serum IgG and IgM antibody reactivity in patients treated with 50 μg Foralumab. Heatmap representing the mean IgG (FIG. 10A) and IgM (FIG. 10B) antibody reactivity in serum samples from patients. FIG. 10C is a volcano plot representing differential IgG and IgM antibody reactivity. Cut-off criteria was defined as p-value<0.05 and log 2 fold change>1 or <−1.

FIG. 11 shows the patient disposition.

FIG. 12 shows a Treg heat map. The DEG found between baseline (T1) and T2 in CD4+CD25 hi CD127 low Tregs are shown for naïve Tregs and activated (memory) Tregs.

FIGS. 13A-13D show the relationship of differentially expressed genes in CD8+ TEMRA to immune function. The functional properties of the DEGs identified were sourced from the literature and assigned to one of twelve groups (FIGS. 13A and 13B). FIG. 13C shows genes that were downregulated from T1 to T2 after treatment. FIG. 13D shows genes that were upregulated from T1 to T2 after treatment. Dark grey indicates a pro inflammatory role for a specific immune function. Light grey indicates an anti-inflammatory role for a specific immune function. As shown in FIG. 13C, 17/19 genes with an pro inflammatory role were downregulated with treatment whereas in FIG. 13D, 16/24 genes with an anti-inflammatory role were upregulated.

FIG. 14 shows PET images showing standardized uptake value ratio images in patient 2 (EA2) demonstrating a significant reduction in [F-18]PBR06 uptake after 3 months of treatment with intranasal Foralumab. Widespread reduction is seen in cortex, thalamus, white matter and cerebellum.

FIG. 15 is a graph showing reduction PET signal in EA2 after 3 months of treatment with nasal Foralumab.

FIG. 16 is a graph showing EDSS scores for patient 2 (EA2).

FIG. 17 is a graph showing a timed 25 foot walk of patient 2 (EA2).

FIG. 18 is a graph showing EDSS and pyramidal scores in patient 1 (EA1) over time.

FIG. 19 is a graph showing the time that patient 1 took to walk 25 feet over time.

FIGS. 20A and 20B show levels of microglial activation in patient 1 over time. FIG. 20A shows PET images, and FIG. 20B shows a quantification of the activated microglial cell PET signal (SUVR-1).

FIGS. 21A-21D show the levels of IL-6, IL-1, INF-γ, and IL-18, respectively, in patient 1 over time.

FIG. 22 is a graph showing EDSS and pyramidal scores in patient 2 (EA2) over time.

FIG. 23 is a graph showing the time patient 2 took to walk 25 feet over time.

FIGS. 24A-I. Nasal anti-CD3 ameliorates pathological outcomes in CCI model of TBI. FIG. 24A: Visual representing experimental timeline of the nasal anti-CD3 treatment and time-points (stars) post CCI and the histopathological experiments that were conducted. FIG. 24B: Brain edema was analyzed on day 3 post-TBI and % water content was measured between the ipsilateral and contralateral hemispheres by Student's t-test. FIG. 24C: Magnetic resonance imaging (MRI) was then performed using 3-Tesla MRI to measure the parenchymal lesion volume at 7 days post-CCI Serial MRI images were taken of TBI-aCD3 and TBI-Iso at 7 days post-CCI. FIG. 24D: MRI lesion volume at 7 days post-TBI was analyzed by Student's t-test. FIG. 24E: Brain sections were stained with hematoxylin and eosin (H&E) at one-month post-TBI and lesion volume was measured by image J software and analyzed by Student's t-test. FIG. 24F: CD11 b+Ly6Chi classical monocytes were examined from the Ipsilateral hemisphere of the brain by fluorescence-activated cell sorting (FACS) at 5 days post TBI between sham-Iso, TBI-Iso, and TBI-aCD3 groups. Statistical analysis by one-way ANOVA, followed by Tukey post hoc analysis. FIG. 24G: Brain sections one-month post-TBI were stained with Iba-1 antibody and were co-stained with DAPI and the % area covered by Iba-1 positive cells was quantified by Image J and analyzed by one-way ANOVA, followed by Tukey post hoc analysis. FIG. 24H: Brain sections 7 days post-TBI were stained with TUNEL and were co-stained with 7-AAD and DAPI. Representative images were captured at 20× magnification at the peri-contusional cortex. 5 sections of each sample were prepared and the area around the contusion was captured and the number of TUNEL positive cells around the contusion were quantified by Image J and analyzed by student-t test. FIG. 24I: Total CD4+, CD4+ Foxp3+ and CD4+ LAP+ Tregs were examined from the Ipsilateral hemisphere of the brain and cervical lymph nodes by fluorescence-activated cell sorting (FACS) at 7 days post-TBI between Sham-Iso, TBI-Iso, and TBI-aCD3 groups. Statistical analysis by one-way ANOVA, followed by Tukey post hoc analysis. n=4-5 mice/group was used for all of the experiments and data is presented as (mean and SEM). *P<0.05, **P<0.01, ***P<0.001, n.s.=not significant. Scale bars are 1 mm (FIGS. 24G and 24E) and 500 μm (FIG. 24H) for full tissue sections. 200 μm for peri-contusional regions and 100 μm for zoomed in sections in. (g) All images were captured at 20× magnification.

FIG. 25A-25H. Nasal anti-CD3 improves behavioral outcomes in CCI model of TBI. FIG. 25A: Visual representing the treatment regimens of a moderate CCI model of TBI (Depth: 1 mm, Diameter: 1.5 mm of the impact tip) with both early and delayed nasal anti-CD3 regimens. FIGS. 25B and 25C: Behavioral testing of rotarod, Morris water maze, probe trial, anxiety like behavior, and locomotor activity that is measured by the open field was assessed between Sham-Iso, TBI-Iso, and TBI-aCD3 groups in two independent cohorts that were given an early and delayed treatment regimen. FIG. 25D: Visual representing the treatment regimen timeline of a severe CCI model of TBI (Depth: 1.5 mm, Diameter: 3.0. mm of the impact tip) with early nasal anti-CD3 regimen. FIG. 25E: Behavioral testing of rotarod, Morris water maze, probe trial, anxiety like behavior, and locomotor activity that is measured by the open field was assessed between Sham-Iso, TBI-Iso, and TBI-aCD3 groups. The Morris water maze was analyzed by two-way ANOVA and the other behavioral tests were analyzed by one-way ANOVA, followed by Tukey post hoc analysis. n=8-12 mice/group was used for all of the experiments and data is presented as (mean and SEM). *P<0.05, **P<0.01, ***P<0.001, ****P<0.0001, n.s.=not significant. FIG. 25F: Relative expression of microglia, astrocyte, oligodendrocyte, and neuronal markers by bulk RNA-seq in 4D4+ microglia isolated Sham-Iso animals (n=5). FIG. 25G: GO Biological Process (BP) pathways significantly up-regulated for TBI-aCD3 vs. Sham-Iso and TBI-Iso vs Sham-Iso at 7-days timepoint. Data is represented by log 2 fold change. Only significant GOBP pathways (P<0.05) from either comparison (TBI-aCD3 vs. Sham-Iso or TBI-Iso vs Sham) are shown. Pathway analysis was performed using GAGE. FIG. 25H: Visual presenting the experimental timeline for the in-vivo phagocytosis functional study (FIG. 26I).

FIGS. 26A-26L: Nasal anti-CD3 modulates acute and chronic microglial response after TBI. FIG. 26A: Visual representing two independent experiments showing 4D4+ ly6C-microglia isolation and bulk-RNA sequencing at 7 days and 1 month after TBI and nasal anti-CD3 treatment. FIG. 26B: DiVenn plot showing the unique and shared differentially expressed genes (P<0.05) between TBI-Iso vs. Sham-Iso and TBI-aCD3 vs. Sham-Iso groups at both 1 week and 1 month post-TBI. Directionality of gene expression is determined by log 2-foldchanges of pairwise gene expression comparisons. FIG. 26C: Top 1000 differentially expressed genes (P<0.05) across all different groups (Sham-Iso, TBI-Iso, and TBI-aCD3) for each timepoint: 7 days (left panel) and 1 month (right panel). FIG. 26D: GO Biological Process (BP) pathways significantly up-regulated for TBI-aCD3 vs. Sham-Iso and TBI-Iso vs Sham-Iso at 1 month timepoint. Data is represented by log 2 fold change. Only significant GOBP pathways (P<0.05) from either comparison (TBI-aCD3 vs. Sham-Iso or TBI-Iso vs Sham-Iso) are shown. Pathway analysis was performed using GAGE. FIG. 26E: Genes significant at either 7 days or 1 month encoding the microglial core sensome in the TBI-aCD3 group relative to the TBI-Iso group at 7-days (left panel) and 1 month (right panel). Differentially expressed genes at each time point are bolded and indicated by an asterisk (P<0.05). Each gene is colored based on their function in the microglial core sensome. The preselected list of microglia sensome genes were obtained from the literature (Faul et al. Handb Clin Neurol 127, 3-13 (2015)). FIGS. 26F and 26G: Heatmaps depicting relative expression levels of genes at two timepoints (7 days and 1 month) involved in the following pathways: phagocytosis and proinflammatory response. FIG. 26H: DAM/MGnD heatmap at 1 month depicting relative expression levels of genes. Bolded genes with an asterisk (*) indicate differentially expressed genes (P<0.05). The preselected sets of gene part of the DAM/MGnd, proinflammatory, and phagocytosis pathways were extracted from the literature (Krasemann et al. Immunity 47, 566-581 e569 (2017); Keren-Shaul et al. Cell 169, 1276-1290 e1217 (2017)). FIG. 26I: In-vivo phagocytosis functional experiment where mice were injected with either labelled apoptotic neurons or DPBS. Gating strategy showing phagocytic positive microglia in TBI-Iso and TBI-aCD3 animals. Visual presenting the experimental timeline of the study is found in FIG. 25H. Data is presented as violin plot and n=5 animals per group were used. Animals that received DPBS are not shown FIG. 26I. Bar plots of Quantitative PCR of the ipsilateral hemisphere at 7 days and 1 month post-TBI. Expression was normalized to GAPDH and presented relative to that of Sham-Iso animals. The significant expression of IL-10 (7 days), Il6, IFNg, Tnf Il17, and Bdnf (1 month) are shown. N=6-8 mice/group (mean and SEM). Statistical analysis by one-way ANOVA, followed by Tukey post hoc analysis. *P<0.05, n.s.=not significant. Quantitative PCR bar plots of significant cytokines (FIG. 26K) and non-significant cytokines (FIG. 26L) of the ipsilateral hemisphere between the Sham-Iso, TBI-Iso, and TBI-aCD3 groups at 7 days and 1 month post-TBI. Expression was normalized to GAPDH. n=6-8 mice/group (mean and SEM). Statistical analysis by one-way ANOVA, followed by Tukey post hoc analysis. *P<0.05, **P<0.01, ***P<0.001, n.s.=not significant.

FIGS. 27A-27J. Nasal anti-CD3 function improves behavioral outcomes after TBI in an IL-10 dependent manner. FIG. 27A: Heatmap shows the relative expression of IL-10 pathway genes at 7 days and 1 month post-CCI in Sham-Iso, TBI-Iso, and TBI-aCD3 groups. The IL-10 pathway genes list was mined from the literature (Xue et al. Immunity 40, 274-288 (2014)). FIG. 27B: IL-10 expression was analyzed by flow cytometry in CD4+, FoxP3+, FoxP3−, LAP+, LAP−, FCRLS+ microglia (Butovsky et al. Nat Neurosci 17, 131-143 (2014, NK1.1+, Ly6C+, and Ly6G+ populations at 7 days post TBI and nasal anti-CD3 treatment. N=4-5 mice/group (mean and SEM). Statistical analysis by one-way ANOVA, followed by Tukey post hoc analysis. FIG. 27C: Visual representing experimental timeline of the nasal anti-CD3 and anti-IL-10 receptor blocking mAbs (aIL-10 R) treatments and time-points (stars) post CCI and the behavioral studies that were conducted. Injection of anti-IL-10 receptor blocking mAbs (aIL-10 R) (0.5 mg/mouse) intraperitoneally every third day (black arrows). FIG. 27D: Behavioral testing of rotarod, Morris water maze, probe trial, anxiety like behavior, and locomotor activity that is measured by the open field was assessed in Sham-Iso, TBI-Iso, TBI-aCD3 and TBI-aCD3-anti-IL10 groups. The Morris water maze was analyzed by two-way ANOVA and the other behavioral tests were analyzed by one-way ANOVA, followed by Tukey post hoc analysis. n=8 mice/group. FIG. 27E: Top 1000 differentially expressed genes (P<0.05) across all different groups (Sham, TBI-Iso, TBI-aCD3, and TBI-aCD3-aIL10R). FIG. 27F: GO Biological Process (BP) pathways significantly down- or up-regulated for TBI-aCD3 vs. Sham-Iso, TBI-Iso vs Sham-Iso and, TBI-aCD3-aIL10R vs. Sham-Iso (gold) at 1 month timepoint. Data is represented by log 2 fold change. Only significant GOBP pathways (P<0.05) from any of the three comparisons (TBI-aCD3 vs. Sham-Iso, TBI-Iso vs Sham or TBI-aCD3-aIL10R vs. Sham-Iso) are shown. Pathway analysis was performed using GAGE. FIG. 27G: Visual representing the microglia and Treg trans-well co-culture. 4D4+ ly6C− microglia was isolated 24 hours post-TBI and was cultured for 7 days. Co-currently a different cohort of TBI animals were treated with nasal aCD3 or the isotype control for 7 days and had their splenic total CD4+ cells sorted and placed on the top of the microglia trans-well culture. The trans-well system was left for 72 hours before the microglia was lysed and analyzed by quantitative PCR. FIG. 27H: Quantitative PCR heatmap of microglia from TBI-Iso total CD4 and TBI-aCD3 total CD4 with expression normalized to GAPDH and analyzed by Student's t-test. n=3 wells per group and each well had 200,000 microglia cells. The total CD4+ in each insert was 800,000 cells that were pooled from 3 biological replicate animals. The expression of Il10, Tgfb1, CD206, and CD14 are presented as bar graphs (mean and SEM). *P<0.05, **P<0.01, ***P<0.001, ****P<0.0001, n.s.=not significant. FIG. 27I: DiVenn plot showing the unique and shared microglial differentially expressed genes (P<0.05) between TBI-Iso vs. Sham-Iso, TBI-aCD3 vs. Sham-Iso, and TBI-aCD3+aIL10R vs. Sham-Iso group comparisons. Directionality of gene expression is determined by log 2-foldchanges of pairwise gene expression comparisons. FIG. 27J Quantitative PCR barplots of microglia between TBI-Iso total CD4+ and TBI-aCD3 total CD4+ after 72 hours of transwell co-culture (FIG. 27G); expression normalized to GAPDH and analyzed by Student's t-test (mean and SEM). n=3 wells per group and each well had 200,000 microglia cells. A total of 800,000 CD4+ cells were pooled from 3 biological replicate animals was used in each insert. n.s.=not significant.

FIGS. 28A-28I. CD4+FoxP3+ regulatory T-cells ameliorate microglial response and improved behavioral outcomes post TBI. FIG. 18A: Visual representing experimental timeline of adoptive transfer experiment. Splenic CD4+ from isotype and aCD3 treated animals (CD45.2) animal and CD4+ FoxP3GFP negative population from aCD3 treated animals (CD45.2) 7 days post-TBI were intraperitoneally transferred into untreated but CCI-injured (CD45.1) mice. The adoptive transfer was done at 3 different timepoints with each animal receiving 2.5 million cells per injection. FIG. 28B: Behavioral testing of rotarod, Morris water maze, probe trial, anxiety like behavior, and locomotor (measured by the open field) was assessed between WT Sham (DPBS treated, baseline), Iso-total CD4+, aCD3-total CD4+, and aCD3-FoxP3(−)GFP groups. The Morris water maze was analyzed by two-way ANOVA and the other behavioral tests were analyzed by one-way ANOVA, followed by Tukey post hoc analysis. n=8 mice/group. FIG. 28C: Visual representing an independent experiment where splenic CD4+ cells from 7 days treated TBI (CD45.2) animals were injected intraperitoneally into untreated, but CCI-injured (CD45.1) animals and the % CD45.2 cells were analyzed by fluorescence-activated cell sorting (FACS) at 3 days after injection. FIG. 28D: Flow cytometry gating of brain, cervical lymph node, and spleen of (CD45.1) animals showing the percent of CD45.2 cell infiltration. n=5 mice/group and the brain was a pool of 5 ipsilateral hemispheres. FIG. 28E: DiVenn plot showing the unique and shared differentially expressed microglial genes (P<0.05) between aCD3-total CD4+ vs. Iso-total CD4+ and aCD3-FoxP3(−)GFP vs. Iso-total CD4+. Directionality of gene expression is determined by log 2-foldchanges of pairwise gene expression comparisons. FIG. 28F: Top 1000 differentially expressed genes (P<0.05) across Iso-total CD4+, aCD3-FoxP3(−)GFP, and aCD3-total CD4+ groups. FIG. 28G: GO Biological Process pathways significantly down- or up-regulated in aCD3-total CD4+ vs. Iso-total CD4+(red) and aCD3-FoxP3(−)GFP vs. Iso-total CD4+. Data is represented by log 2 fold change. Only the significant GOBP pathways (P<0.05) from either comparison (aCD3-total CD4+ vs. Iso-total CD4+ or aCD3-FoxP3(−)GFP vs. Iso-total CD4+) are shown. Pathway analysis was performed using GAGE. FIG. 28H: Bar plots of quantitative PCR of the ipsilateral hemispheres between Iso-total CD4+, aCD3-FoxP3(−)GFP groups, and aCD3-total CD4+; expression normalized to GAPDH. IL-10, Tgfb1, Il2, Il22, and Gdnf expression at 1-month post TBI and three adoptive transfer experiments are shown. n=5 mice/group (mean and SEM). Statistical analysis by one-way ANOVA, followed by Tukey post hoc analysis. *P<0.05, **P<0.01, n.s.=not significant. FIG. 28I: Quantitative PCR bar plots shows cytokines expression in the ipsilateral hemisphere of aCD3-total CD4+, aCD3-FoxP3(−) GFP, and Iso-total CD4+ adoptive transfer experiment groups at 1-month post TBI. n=5 mice/group (mean and SEM). *P<0.05, n.s.=not significant.

### DETAILED DESCRIPTION OF THE INVENTION

The methods and compositions described herein are based, in part, upon the discovery that the inflammatory phenotype of microglial cells is modulated by anti-CD3 antibodies. Specifically, it was discovered that CD74, the invariant chain involved in MHC II presentation and H2-AB1, a MHC II antigen is downregulated in microglia upon anti-CD3 administration. Critically, anti-CD3 administration not only modulates the gene expression of Clec7+ microglia in APPPS1 mice but also reduced the number of Clec7+ plaque-associated microglia.

More specifically, the methods described herein relate to the reduction microglial activation by reducing CD3 expression.

Microglia are non-neuronal macrophage-like cells present in the developing and adult central nervous systems. Upon neuronal injury, microglia are transformed from a resting state to an activated state, characterized by changes in morphology, immunophenotype, migration, and proliferation. Activated microglia participate in the phagocytosis of neurons, and, furthermore, microglial proteases are involved in neuronal degradation.

The present methods and compounds are useful in preventing, treating, or ameliorating neurological signs and symptoms associated with chronic neurological disease, including but not limited to Multiple Sclerosis (MS), Alzheimer's disease (AD), Lewy Body Disease, Parkinson's Disease (PD), Parkinson's Disease (PD) Huntington's Disease (HD), Amyotrophic Lateral Sclerosis (ALS), epilepsy, HIV-associated encephalopathy and AIDS related dementia.

The present methods are also useful in preventing, treating, or ameliorating the neurological signs and symptoms associated with inflammatory conditions affecting the nervous system including the CNS.

Stated in a different way, the present methods and compounds are useful in preventing, suppressing, or reducing the activation of microglia in the CNS that occurs as a part of acute or chronic CNS disease. The suppression or reduction of microglial activation can be assessed by various methods as would be apparent to those in the art; one such method is to measure the production or presence of compounds that are known to be produced by activated microglia, and compare such measurements to levels of the same compounds in control situations. Alternatively, the effects of the present methods and compounds in suppressing, reducing or preventing microglial activation may be assessed by comparing the signs and/or symptoms of CNS disease in treated and control subjects, where such signs and/or symptoms are associated with or secondary to activation of microglia.

As used herein, the terms “combating”, “treating” and “ameliorating” are not necessarily meant to indicate a reversal or cessation of the disease process underlying the CNS condition afflicting the subject being treated. Such terms indicate that the deleterious signs and/or symptoms associated with the condition being treated are lessened or reduced, or the rate of progression is reduced, compared to that which would occur in the absence of treatment. A change in a disease sign or symptom may be assessed at the level of the subject (e.g., the function or condition of the subject is assessed), or at a tissue or cellular level (e.g., the production of markers of glial activation is lessened or reduced). Where the methods disclosed herein are used to treat chronic CNS conditions (such as Multiple Sclerosis, or MS), the methods may slow or delay the onset of symptoms, while not necessarily affecting or reversing the underlying disease process.

Surprisingly, nasal Foralumab given for 5 consecutive days to healthy subjects was safe at doses of 10 μg, 50 μg and 250 μg. Immune effects were predominantly observed at the 50 μg dose. A dose effect with 50 μg being more immunomodulatory than 250 μg is consistent with animal studies of mucosal tolerance in which higher doses do not induce immune regulation, most likely due to the partial signaling that occurs at intermediate doses which favors the induction of regulatory cells. Importantly, the biologic effect of nasal anti-CD3 is markedly different from that which occurs with IV anti-CD3. IV anti-CD3 is associated with modulation of CD3 from the cell surface, a decrease in CD3 cells and side effects that include cytokine release syndrome and in some instances activation of EBV. EBV reactivation was observed with IV Foralumab at the 500 μg and 1000 μg doses. In contrast, for nasal Foralumab, no EBV activation was observed at any of the doses or modulation of CD3 from the cell surface. Furthermore, when administered nasally, Foralumab was not detected in the bloodstream. Thus, unlike IV administered anti-CD3 which acts systemically by lysing CD3+ T cells, followed by immune reconstitution, nasal anti-CD3 acts locally at the mucosal surface as an immunomodulatory agent. In animal studies, nasal anti-CD3 localized to the cervical lymph nodes and as with human studies, nasally administered anti-CD3 was not detected in the bloodstream of animals.

The in vitro activation properties of Foralumab was compared to a commonly used anti-CD3 monoclonal antibody UCHT1. Foralumab induced preferential CD8+ T cell proliferation and reduced CD4⁺ T cell proliferation. Foralumab stimulation of purined CD4⁺ T cells resulted in higher expression of CTLA4.

Animal studied showed that nasal anti-CD3 induced LAP+IL-10 secreting Tregs that could adoptively transfer protection. However, a prominent increase in IL-10 was not found in human studies. Although increase of DN LAP+ T cells in 50 μg treated subjects at the T4 timepoint was observed. The major effects with nasal Foralumab occurred in CD8+ T cells which is consistent with the effects observed with other anti-CD3 monoclonal antibodies given IV in humans. A reduction of CD8⁺ effector memory cells, an increase in naïve CD8⁺ as well as CD4⁺ cells, and a reduction of CD8+ T cell granzyme B and perform expression was observed. Antigen array studies also showed most prominent effects at the 50 μg dose.

scRNAseq analysis of subjects receiving the 50 μg dose allowed a more detailed analysis of the immune effects of nasal Foralumab. Although some of the DEGs functioned in homeostatic cell biologic processes, most of the affected DEGs had immunologic functions. In the CD8+ population were anti-inflammatory. Interestingly, the upregulation of TIGIT which are associated with IV administration of Teplizumab was observed. Nasal Foralumab treated CD8⁺ TEMRA population had induction of KIR3DL2 in addition to TIGIT, KLTG1 and TGFB1. Similar patterns were observed in non-regulatory CD4⁺ T cells with downregulation of DEGs associated with activated subsets. Upregulated genes in CD4⁺ memory cells included CTLA4 and TGFB1, which is consistent with what is observed following in vitro stimulation of T cells by Foralumab. Only minimal changes were observed in the Treg population with only 4 DEGs were identified including reduced expression of JUNB which may enhance Treg stability by inhibiting Th17 differentiation.

Thus, it does not appear that nasal Foralumab is directly expanding classical Tregs. Changes were also observed in monocyte populations including expression of DQ and DP which are associated with T cells that produce higher levels of IL-10. Taken together, nasal anti-CD3 has a strong immunomodulatory effect on the immune response that is dose dependent, decreases inflammation and promotes regulation. In summary, that nasal Foralumab is safe and induces immune effects at a dose of 50 μg given for 5 consecutive days.

### Anti-CD3 Antibodies

The anti-CD3 antibodies can be any antibodies specific for CD3. The term “antibody” as used herein refers to an immunoglobulin molecule or immunologically active portion thereof, i.e., an antigen-binding portion. Examples of immunologically active portions of immunoglobulin molecules include scFv, F(ab) and F(ab′) 2 fragments, which retain the ability to bind CD3. Such fragments can be obtained commercially, or using methods known in the art. For example, F(ab)₂ fragments can be generated by treating the antibody with an enzyme such as pepsin, a non-specific endopeptidase that normally produces one F(ab)₂ fragment and numerous small peptides of the Fc portion. The resulting F(ab)² fragment is composed of two disulfide-connected Fab units. The Fc fragment is extensively degraded and can be separated from the F(ab)₂ by dialysis, gel filtration or ion exchange chromatography. F(ab) fragments can be generated using papain, a non-specific thiol-endopeptidase that digests IgG molecules, in the presence of a reducing agent, into three fragments of similar size: two Fab fragments and one Fc fragment. When Fc fragments are of interest, papain is the enzyme of choice because it yields a 50,00 Dalton Fc fragment; to isolate the F(ab) fragments, the Fc fragments can be removed, e.g., by affinity purification using protein A/G. A number of kits are available commercially for generating F(ab) fragments, including the ImmunoPure IgG1 Fab and F(ab′)2. Preparation Kit (Pierce Biotechnology, Rockford, Ill.). In addition, commercially available services for generating antigen-binding fragments can be used, e.g., Bio Express, West Lebanon, N.H.

The antibody can be a polyclonal, monoclonal, recombinant, e.g., a chimeric, de-immunized or humanized, fully human, non-human, e.g., murine, single chain antibody or single domain antibody. The antibody may be of any class, for example, IgG, IgM, IgA, IgE or IgD. The antibody may also be of any subclass, e.g., IgG₁, IgG₂, IgG₃ and IgG₄ or others. Furthermore, in humans, the light chain may be a kappa chain or a lambda chain. In some embodiments the antibody has effector function and can fix complement. In some embodiments, the antibody has reduced or no ability to bind an Fc receptor. For example, the anti-CD3 antibody can be an isotype or subtype, fragment or other mutant, which does not support binding to an Fc receptor, e.g., it has a mutagenized or deleted Fc receptor binding region. The antibody can be coupled to a toxin or imaging agent.

A number of anti-CD3 antibodies are known, including but not limited to OKT3 (muromonab/Orthoclone OKT3™, Ortho Biotech, Raritan, N.J.; U.S. Pat. No. 4,361,549); hOKT3(1 (Herold et al., N.E.J.M. 346(22):1692-1698 (2002); HuM291 (Nuvion™, Protein Design Labs, Fremont, Calif); gOKT3-5 (Alegre et al., J. Immunol. 148(11):3461-8 (1992); 1F4 (Tanaka et al., J. Immunol. 142:2791-2795 (1989)); G4.18 (Nicolls et al., Transplantation 55:459-468 (1993)); 145-2C11 (Davignon et al., J. Immunol. 141(6):1848-54 (1988)); and as described in Frenken et al., Transplantation 51(4):881-7 (1991); U.S. Pat. Nos. 6,491,9116, 6,406,696, and 6,143,297).

Methods for making such antibodies are also known. A full-length CD3 protein or antigenic peptide fragment of CD3 can be used as an immunogen, or can be used to identify anti-CD3 antibodies made with other immunogens, e.g., cells, membrane preparations, and the like, e.g., E rosette positive purified normal human peripheral T cells, as described in U.S. Pat. Nos. 4,361,549 and 4,654,210. The anti-CD3 antibody can bind an epitope on any domain or region on CD3.

Chimeric, humanized, de-immunized, or completely human antibodies are desirable for applications which include repeated administration, e.g., therapeutic treatment of human subjects.

Chimeric antibodies contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The present disclosure also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The anti-CD3 antibody can also be a single chain antibody. A single-chain antibody (scFV) can be engineered (see, for example, Colcher et al., Ann. N. Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target CD3 protein. In some embodiments, the antibody is monovalent, e.g., as described in Abbs et al., Ther. Immunol. 1(6):325-31 (1994), incorporated herein by reference.

Exemplary anti-CD3 antibodies, comprise a heavy chain complementarity determining region 1 (CDRH1) comprising the amino acid sequence GYGMH (SEQ ID NO: 1), a heavy chain complementarity determining region 2 (CDRH2) comprising the amino acid sequence VIWYDGSKKYYVDSVKG (SEQ ID NO: 3), a heavy chain complementarity determining region 3 (CDRH3) comprising the amino acid sequence QMGYWHFDL (SEQ ID NO: 4), a light chain complementarity determining region 1 (CDRL1) comprising the amino acid sequence RASQSVSSYLA (SEQ ID NO: 5), a light chain complementarity determining region 2 (CDRL2) comprising the amino acid sequence DASNRAT (SEQ ID NO: 6), and a light chain complementarity determining region 3 (CDRL3) comprising the amino acid sequence QQRSNWPPLT (SEQ ID NO: 7).

In some embodiments, the anti-CD3 antibody comprises a variable heavy chain amino acid sequence comprising QVQLVESGGGVVQPGRSLRLSCAASGFKFSGYGMHWVRQAPGKGLEWVAVIWYD GSKKYYVDSVKGRFTISRDNSKNTLYLQMNSLRAEDTAVYYCARQMGYWHFDLW GRGTLVTVSS (SEQ ID NO: 8) and a variable light chain amino acid sequence comprising

(SEQ ID NO: 9)
EIVLTQSPATLSLSPGERATLSCRASQSVSSYLAWYQQKPGQAPR
LLIYDASNRATGIPARFSGSGSGTDFTLTISSLEPEDFAVYYCQQ
RSNWPPLTFGGGTKVEIK.

Preferably, the anti-CD3 antibody comprises a heavy chain amino acid sequence comprising: QVQLVESGGGVVQPGRSLRLSCAASGFKFSGYGMHWVRQAPGKGLEWVAVIW YDGSKKYYVDSVKGRFTISRDNSKNTLYLQMNSLRAEDTAVYYCARQMGYWH FDLWGRGTLVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPEAEGGPSVFLFPPKPKDTLMISRTPEVTCVVVDVSHE DPEVKFNWYVDGVEVHNAKTKPREEQYNSTYRVVSVLTVLHQDWLNGKEYKC KVSNKALPAPIEKTISKAKGQPREPQVYTLPPSREEMTKNQVSLTCLVKGFYPSDI AVEWESNGQPENNYKTTPPVLDSDGSFFLYSKLTVDKSRWQQGNVFSCSVMHE ALHNHYTQKSLSLSPGK (SEQ ID NO: 10) and a light chain amino acid sequence comprising: EIVLTQSPATLSLSPGERATLSCRASQSVSSYLAWYQQKPGQAPRLLIYDASNRAT GIPARFSGSGSGTDFTLTISSLEPEDFAVYYCQQRSNWPPLTFGGGTKVEIKRTVAA PSVFIFPPSDEQLKSGTASVVCLLNNFYPREAKVQWKVDNALQSGNSQESVTEQDS KDSTYSLSSTLTLSKADYEKHKVYACEVTHQGLSSPVTKSFNRGEC (SEQ ID NO: 11). This anti-CD3 antibody is referred to herein as NI-0401, Foralumab, or 28F11-AE. (See e.g., Dean Y, Dépis F, Kosco-Vilbois M. “Combination therapies in the context of anti-CD3 antibodies for the treatment of autoimmune diseases.” Swiss Med Wkly. (2012) (the contents of which are hereby incorporated by reference in its entirety).

In some embodiments, the anti-CD3 antibody is a fully human antibody or a humanized antibody. In some embodiments, the anti-CD3 antibody formulation includes a full length anti-CD3 antibody. In alternative embodiments, the anti-CD3 antibody formulation includes an antibody fragment that specifically binds CD3. In some embodiments, the anti-CD3 antibody formulation includes a combination of full-length anti-CD3 antibodies and antigen binding fragments that specifically bind CD3.

In some embodiments, the antibody or antigen-binding fragment thereof that binds CD3 is a monoclonal antibody, domain antibody, single chain, Fab fragment, a F(ab′)2 fragment, a scFv, a scAb, a dAb, a single domain heavy chain antibody, or a single domain light chain antibody. In some embodiments, such an antibody or antigen-binding fragment thereof that binds CD3 is a mouse, other rodent, chimeric, humanized or fully human monoclonal antibody.

Optionally, the anti-CD3 antibody or antigen binding fragment thereof used in the formulations of the disclosure includes at least one an amino acid mutation. Typically, the mutation is in the constant region. The mutation results in an antibody that has an altered effector function. An effector function of an antibody is altered by altering, i.e., enhancing or reducing, the affinity of the antibody for an effector molecule such as an Fc receptor or a complement component. For example, the mutation results in an antibody that is capable of reducing cytokine release from a T-cell. For example, the mutation is in the heavy chain at amino acid residue 234, 235, 265, or 297 or combinations thereof.

Preferably, the mutation results in an alanine residue at either position 234, 235, 265 or 297, or a glutamate residue at position 235, or a combination thereof.

Preferably, the anti-CD3 antibody provided herein contains one or more mutations that prevent heavy chain constant region-mediated release of one or more cytokine(s) in vivo.

In some embodiments, the anti-CD3 antibody or antigen binding fragment thereof used in the formulations of the disclosure is a fully human antibody. The fully human CD3 antibodies used herein include, for example, a L²³⁴ L²³⁵→A²³⁴ E²³⁵ mutation in the Fc region, such that cytokine release upon exposure to the anti-CD3 antibody is significantly reduced or eliminated. The L²³⁴ L²³⁵→A²³⁴ E²³⁵ mutation in the Fc region of the anti-CD3 antibodies provided herein reduces or eliminates cytokine release when the anti-CD3 antibodies are exposed to human leukocytes, whereas the mutations described below maintain significant cytokine release capacity. For example, a significant reduction in cytokine release is defined by comparing the release of cytokines upon exposure to the anti-CD3 antibody having a L²³⁴ L²³⁵→A²³⁴ E²³⁵ mutation in the Fc region to level of cytokine release upon exposure to another anti-CD3 antibody having one or more of the mutations described below. Other mutations in the Fc region include, for example, L²³⁴ L²³⁵→A²³⁴, A²³⁵, L²³⁵ 4 E²³⁵, N²⁹⁷→A²⁹⁷, and D²⁶⁵→A²⁶⁵.

The term “cytokine” refers to all human cytokines known within the art that bind extracellular receptors expressed on the cell surface and thereby modulate cell function, including but not limited to IL-2, IFN-gamma, TNF-α, IL-4, IL-5, IL-6, IL-9, IL-10, and IL-13.

### Pharmaceutical Compositions

The anti-CD3 antibodies described herein can be incorporated into a pharmaceutical composition suitable for mucosal administration, e.g., by inhalation, or absorption, e.g., via nasal, intranasal, or pulmonary administration.

For the purpose of mucosal therapeutic administration, the active compound (e.g., an anti-CD3 antibody) can be incorporated with excipients or carriers suitable for administration by inhalation or absorption, e.g., via nasal sprays or drops. For nasal administration, the formulations may be an aerosol in a sealed vial or other suitable container.

The pharmaceutical compositions and mucosal (e.g. nasal) dosage forms can further comprise one or more compounds that reduce the rate by which an active ingredient will decompose. Thus, the mucosal dosage forms described herein can be processed into an immediate release or a sustained release dosage form. Immediate release dosage forms may release the anti-CD3 antibody in a fairly short time, for example, within a few minutes to within a few hours. Sustained release dosage forms may release the anti-CD3 antibody over a period of several hours, for example, up to 24 hours or longer, if desired. In either case, the delivery can be controlled to be substantially at a certain predetermined rate over the period of delivery.

Nasal delivery is considered an attractive route for needle-free, systemic drug delivery, especially when rapid absorption and effect are desired. In addition, nasal delivery may help address issues related to poor bioavailability, slow absorption, drug degradation, and adverse events (AEs) in the gastrointestinal tract and avoids the first-pass metabolism in the liver.

Liquid nasal formulations are mainly aqueous solutions, but suspensions and emulsions can also be delivered. In traditional spray pump systems, antimicrobial preservatives are typically required to maintain microbiological stability in liquid formulations.

Metered spray pumps have dominated the nasal drug delivery market since they were introduced. The pumps typically deliver about 25-200 μL per spray, and they offer high reproducibility of the emitted dose and plume geometry. The particle size and plume geometry can vary within certain limits and depend on the properties of the pump, the formulation, the orifice of the actuator, and the force applied. Traditional spray pumps replace the emitted liquid with air, and preservatives are therefore required to prevent contamination.

Alternative spray systems that avoid the need for preservatives can also be used. These systems use a collapsible bag, a movable piston, or a compressed gas to compensate for the emitted liquid volume. The solutions with a collapsible bag and a movable piston compensating for the emitted liquid volume offer the additional advantage that they can be emitted upside down, without the risk of sucking air into the dip tube and compromising the subsequent spray. his may be useful for some products where the patients are bedridden and where a head down application is recommended. Another method used for avoiding preservatives is that the air that replaces the emitted liquid is filtered through an aseptic air filter. In addition, some systems have a ball valve at the tip to prevent contamination of the liquid inside the applicator tip.

The kits described herein can include an anti-CD3 antibody composition as an already prepared liquid oral or mucosal dosage (e.g. nasal) form ready for administration or, alternatively, can include an anti-CD3 antibody composition as a solid pharmaceutical composition that can be reconstituted with a solvent to provide a liquid oral dosage form or mucosal dosage form. When the kit includes an anti-CD3 antibody composition as a solid pharmaceutical composition that can be reconstituted with a solvent to provide a liquid dosage form (e.g., for oral or nasal administration), the kit may optionally include a reconstituting solvent. In this case, the constituting or reconstituting solvent is combined with the active ingredient to provide a liquid oral dosage form of the active ingredient.

Typically, the active ingredient is soluble in the solvent and forms a solution. The solvent can be, e.g., water, a non-aqueous liquid, or a combination of a non-aqueous component and an aqueous component. Suitable non-aqueous components include, but are not limited to oils; alcohols, such as ethanol; glycerin; and glycols, such as polyethylene glycol and propylene glycol. In some embodiments, the solvent is phosphate buffered saline (PBS).

For administration by inhalation, the mucosal anti-CD3 antibody compounds can be delivered in the form of an aerosol spray from pressured container or dispenser that contains a suitable propellant, e.g., a gas such as carbon dioxide, or a nebulizer. Such methods include those described in U.S. Pat. No. 6,468,798.

In one embodiment, the mucosal anti-CD3 antibody compositions are prepared with carriers that will protect the anti-CD3 antibody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811

Dosage, toxicity and therapeutic efficacy of such anti-CD3 antibody compositions can be determined by standard pharmaceutical procedures in cell cultures (e.g., of cells taken from an animal after mucosal administration of an anti-CD3 antibody) or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions which exhibit high therapeutic indices are preferred. While anti-CD3 antibody compositions that exhibit toxic side effects may be used, care should be taken to design a delivery system that targets such compounds to the site of affected tissue in order to minimize potential damage and, thereby, reduce side effects.

The data obtained from the cell cultures (e.g., of cells taken from an animal after mucosal administration of an anti-CD3 antibody) and animal studies can be used in formulating a range of dosage for use in humans. The dosage of anti-CD3 antibody compositions lies preferably within a range of circulating concentrations that include the ED₅₀ with little or no toxicity. The dosage may vary within this range depending upon the dosage form employed and the route of administration utilized. For any oral or mucosal anti-CD3 antibody compositions used in the methods described herein, the therapeutically effective dose can be estimated initially from assays of cell cultures (e.g., of cells taken from an animal after mucosal administration of an anti-CD3 antibody). A dose may be formulated in animal models to achieve a desired circulating plasma concentration of IL-10 or TGFβ, or of regulatory cells, in the range that includes the IC₅₀ (i.e., the concentration of the test compound which achieves a half-maximal inhibition of symptoms) as determined in cell culture. Such information can be used to more accurately determine useful doses in humans. Levels of IL-10 or TGFβ. in plasma can be measured by methods known in the art, for example, by ELISA. Levels of regulatory cells can be measured by methods known in the art, for example, by flow cytometry-based methods.

As defined herein, a therapeutically effective amount of an anti-CD3 antibody (i.e., an effective dosage) depends on the antibody selected, the mode of delivery, and the condition to be treated. For instance, single dose amounts may be in the range of about between 5-200 μg; about between 25-175 μg; about between 25-100; μg about between 10-150 μg; about between 5-100 μg; about between 5-50 μg; about between 10-50 μg; about between 5-50 μg; about between 25-75 μg. In some embodiments, the single dose is about 5, 10, 15, 20, 25, 30, 35, 40, 45, 50, 55, 60, 65, 70, 75, 80, 85, 90, 95 100, 105, 110, 115, 120, 125, 130, 135, 140, 145, 150, 155, 160, 165, 170, 175, 180, 185, 190, 195, and 200 μg. Preferably the daily dose is 50 μg per day. The daily dose may be administered via a single nostril.

Alternatively, the daily dose may be split equally between both nostrils

As used herein, “dosing regimen” or “dosage regimen” refers to the amount of agent, for example, the composition containing an anti-CD3 antibody, administered, and the frequency of administration. The dosing regimen is a function of the disease or condition to be treated, and thus can vary.

As used herein, “frequency” of administration refers to the time between successive administrations of treatment. For example, frequency can be days, weeks or months. For example, frequency can be more than once weekly, for example, twice a week, three times a week, four times a week, five times a week, six times a week or: daily. Frequency also can be one, two, three or four weeks. The particular frequency is a function of the particular disease or condition treated. Generally, frequency is more than once weekly, and generally is three times weekly.

The anti-CD3 antibody compositions can be administered from one or more times per day to one or more times per week; including once every other day. For example, the anti-CD3 antibody composition is administered once daily every other day for a period of one, two, three, four or more weeks.

As used herein, a “cycle of administration” refers to the repeated schedule of the dosing regimen of administration of anti-CD3 antibody that is repeated over successive administrations. A cycle can be a week, two weeks, three weeks or four weeks. For example, an exemplary cycle of administration is a 2 week cycle. The subject may receive between one and ten cycles of administration. The subject may review one, two three, four five or more cycles of administration. Optionally, a drug holiday is given between cycles of administration. The Drug holiday can be 1 to 4 weeks. Preferably the drug holiday is one week

As used herein, “unit dose form” or “unit dosage form” refers to physically discrete units suitable for human and animal subjects and packaged individually as is known in the art.

The anti-CD3 antibody compositions can be administered from one or more times per day to one or more times per week; including once every other day. For example, the anti-CD3 antibody composition is administered once daily every other day for a period of one, two, three, four or more weeks.

The oral or mucosal anti-CD3 antibody compositions can be administered, e.g., for about 10 to 14 days or longer. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds can include a single treatment or, can include a series of treatments.

The oral or mucosal anti-CD3 antibody compositions can also include one or more therapeutic agents useful for treating an autoimmune disorder. Such therapeutic agents can include, e.g., NSAIDs (including COX-2 inhibitors); other antibodies, e.g., anti-cytokine antibodies, e.g., antibodies to IFN-α, IFN γ and/or TNFα; gold-containing compounds; immunosuppressive drugs (such as corticosteroids, e.g., prednisolone and methyl prednisolone; cyclophosphamide; azathioprine; mycophenolate mofetil (MMF); cyclosporin and tacrolimus; methotrexate; or cotrimoxazole); heat shock proteins (e.g., as described in U.S. Pat. No. 6,007,821); and treatments for MS, e.g., .beta.-interferons (e.g., interferon β-1a, interferon β1b), mitoxantrone, or glatiramer acetate.

The pharmaceutical compositions can be included in a container, pack, or dispenser together with instructions for administration.

### Methods of Treatment

The mucosal (e.g. nasal) anti-CD3 antibody compositions described herein can be administered to a subject to treat, or alleviate a sign or symptom of disorders associated microglial activation. In some embodiments, the mucosal (e.g. nasal) anti-CD3 antibody compositions described herein can be administered to a subject to prevent disorders associated microglial activation. In some embodiments the anti-CD3 is administered at a dose described herein, for example, a single dose amount in the range of about between 5-200 μg; about between 25-175 μg; about between 25-100; μg about between 10-150 μg; about between 5-100 μg; about between 5-50 μg; about between 10-50 μg; about between 5-50 μg; about between 25-75 μg. For example, the single dose may be about 5, 10, 15, 20, 25, 30, 35, 40, 45, 50, 55, 60, 65, 70, 75, 80, 85, 90, 95 100, 105, 110, 115, 120, 125, 130, 135, 140, 145, 150, 155, 160, 165, 170, 175, 180, 185, 190, 195, and 200 μg. In some embodiments, the daily dose is 10-200 μg per day. In some embodiments, the daily dose is 50 μg per day. The daily dose may be administered via a single nostril.

Examples of disorders associated microglial activation include for example, a neurodegenerative disorder, an ischemic related disease or injury, traumatic brain injury or a lysosomal storage disease. Ischemic related disease but are not limited to, an ischemic-reperfusion injury, stroke, and myocardial infarction. The ischemic-reperfusion injury incudes injury to lung tissue, cardiac tissue, or neuronal tissue. Traumatic brain injuries includes, but are not limited to concussion such as is a repetitive concussive injury or whiplash.

Neurodegenerative disorders include for example, Multiple Sclerosis (MS) (e.g., relapse-remitting MS and secondary-progressive MS), Alzheimer's disease (AD), Lewy Body Disease, Parkinson's Disease (PD), Parkinson's Disease (PD) Huntington's Disease (HD), Amyotrophic Lateral Sclerosis (ALS), epilepsy, HIV-associated encephalopathy and AIDS related dementia.

The mucosal (e.g. nasal) anti-CD3 antibody compositions described herein can be administered to a subject to treat disorders associated with neural inflammation. Neural inflammation is often associated with neurodegenerative diseases, including, for example, AD, PD, MS, and ALS. Levels of neural inflammation may be determined using imaging techniques such as MRI and PET. In some embodiments the anti-CD3 is administered at a dose described herein, for example, a single dose amount in the range of about between 5-200 μg; about between 25-175 μg; about between 25-100; μg about between 10-150 μg; about between 5-100 μg; about between 5-50 μg; about between 10-50 μg; about between 5-50 μg; about between 25-75 μg. For example, the single dose may be about 5, 10, 15, 20, 25, 30, 35, 40, 45, 50, 55, 60, 65, 70, 75, 80, 85, 90, 95 100, 105, 110, 115, 120, 125, 130, 135, 140, 145, 150, 155, 160, 165, 170, 175, 180, 185, 190, 195, and 200 μg. In some embodiments, the daily dose is 10-200 μg per day. In some embodiments, the daily dose is 50 μg per day. The daily dose may be administered via a single nostril.

In some embodiments, the present methods result in a reduction in the levels of neural inflammation in the subject of at least 10%, at least 20%, at least 30%, at least 40%, at least 50%, at least 60%, at least 70%, at least 80%, at least 90%, or at least 95% compared to the levels of neural inflammation prior to the administration of the anti-CD3 antibody. In some embodiments, the present methods result in a reduction in neural inflammation in the subject of 5-10%, 10-20%, 20-30%, 30-40%, 40-50%, 50-60%, 60-70%, 70-80%, 80-90%, 90-95%, or 95-100% compared to the levels of neural inflammation prior to the administration of the anti-CD3 antibody. Neural inflammation may be assessed after any suitable time of treatment, for example, after 1 week, after 2 weeks, after 3 weeks, after 4 weeks, after 5 weeks, after 6 weeks, after 7 weeks, after 8 weeks, after 3 months, after 6 months, after 9 months, after 12 months, after 18 months, after 2 years, after 3 years, or after 5 years of treatment. In some embodiment, the reduction in neural inflammation persists through a washout period (e.g., a 1-week, 2-week, 3-week, 4-week, 5-week, 6-week, 7-week, 8-week, 9-week, or 12-week washout period). Neural inflammation may be determined for example, in the whole brain, in the cerebral cortex region of the brain, in the thalamus region of the brain, in the white matter of the brain, and/or in the cerebellum region of the brain.

In some embodiments, a therapeutically effective amount of a mucosal (e.g. nasal) anti-CD3 antibody composition can be, e.g., the amount necessary to reduce microglial activation by about at least 20%. In some embodiments, microglial activation is reduced by at least about 30%, about 40%, about 50%, about 60%, about 70% about 80%, or about 90% from pre-treatment levels.

In some embodiments microglial activation is reduced by at least about by at least about 20%, about 25%, about 30%, about 35%, about 40%, about 45%, about 50%, about 55%, about 60%, about 65%, about 70%, about 75%, about 80%, about 85%, about 90%, or about 95% from pre-treatment levels (i.e., baseline) in the whole brain.

In some embodiments microglial activation is reduced by at least about by at least about 20%, about 25%, about 30%, about 35%, about 40%, about 45%, about 50%, about 55%, about 60%, about 65%, about 70%, about 75%, about 80%, about 85%, about 90%, or about 95% from pre-treatment levels (i.e., baseline) in the cerebral cortex region of the brain.

In some embodiments microglial activation is reduced by at least about by at least about 20%, about 25%, about 30%, about 35%, about 40%, about 45%, about 50%, about 55%, about 60%, about 65%, about 70%, about 75%, about 80%, about 85%, about 90%, or about 95% from pre-treatment levels (i.e., baseline) in the thalamus region of the brain.

In some embodiments microglial activation is reduced by at least about by at least about 20%, about 25%, about 30%, about 35%, about 40%, about 45%, about 50%, about 55%, about 60%, about 65%, about 70%, about 75%, about 80%, about 85%, about 90%, or about 95% from pre-treatment levels (i.e., baseline) in the white matter of the brain.

In some embodiments microglial activation is reduced by at least about by at least about 20%, about 25%, about 30%, about 35%, about 40%, about 45%, about 50%, about 55%, about 60%, about 65%, about 70%, about 75%, about 80%, about 85%, about 90%, or about 95% from pre-treatment levels. (i.e., baseline) in the cerebellum region of the brain.

Reduction of microglial activation is sustained for 1 week, 2 weeks, 3 weeks, 4 weeks, 5 weeks, 6 weeks, 7 weeks, 8 weeks or more after cessation of treatment.

Reduction of microglial activation may be sustained for 1 month, 2 months, 3 months, 4 months, 5 months, 6 months, 7 months, 8 months, 9 months, 10 months, 11 month, 12 months or more after cessation of treatment.

In some embodiments, the present methods result in a reduction in microglial activation in the subject of 5-10%, 10-20%, 20-30%, 30-40%, 40-50%, 50-60%, 60-70%, 70-80%, 80-90%, 90-95%, or 95-100% compared to the levels of microglial activation prior to the administration of the anti-CD3 antibody. Microglial activation may be determined by any suitable method known in the art, including, for example, PET scans such as those described herein. Microglial activation may be assessed after any suitable time of treatment, for example, after 1 week, after 2 weeks, after 3 weeks, after 4 weeks, after 5 weeks, after 6 weeks, after 7 weeks, after 8 weeks, after 3 months, after 6 months, after 9 months, after 12 months, after 18 months, after 2 years, after 3 years, or after 5 years of treatment. In some embodiment, the reduction in microglial activation persists through a washout period (e.g., a 1-week, 2-week, 3-week, 4-week, 5-week, 6-week, 7-week, 8-week, 9-week, or 12-week washout period). Microglial activation may be determined for example, in the whole brain, in the cerebral cortex region of the brain, in the thalamus region of the brain, in the white matter of the brain, and/or in the cerebellum region of the brain.

In addition, concentrations of TGF-β1 can be measured. For example, TGF-β1 are measured in the peripheral blood, e.g., using an enzyme-linked immunosorbent assay (ELISA) or a cell-based assay such as FACS scanning, to monitor the induction of tolerance. In some embodiments, a therapeutically effective amount of an oral or mucosal anti-CD3 antibody composition is the amount necessary increase levels of cells secreting TGF-β1 by about 20% or more. In some embodiments, levels of cells secreting TGF-β1 are increased by at least about 60%, 70%, 80%, 90%, or 100%, e.g., doubled.

In addition, cellular expression of CD74, H2-Ab and/or CX3CR1 can be measured. In some embodiments, a therapeutically effective amount of an oral or mucosal anti-CD3 antibody composition is the amount necessary decrease the expression levels of CD74 and/or H2-Ab-1 by about 20% or more. In some embodiments, levels of expression of CD74 and/or H2-Ab-1 are decreased by at least about 60%, 70%, 80%, 90%, or 100%, e.g., halved.

In some embodiments, a therapeutically effective amount of mucosal anti-CD3 antibody composition is the amount necessary increase the expression levels of CX3CR1 by about 20% or more. In some embodiments, levels of expression of CX3CR1 is increased by at least about 60%, 70%, 80%, 90%, or 100%, e.g., doubled

Furthermore, cellular expression of CX3CR1 and/or CCR2 on Ly6C^{high} splenocytes can be measured. In some embodiments, a therapeutically effective amount of an oral or mucosal anti-CD3 antibody composition is the amount necessary increases the expression levels of CX3CR1 and/or CCR2 on Ly6C^{high} splenocytes by about 20% or more. In some embodiments, levels of expression of CX3CR1 and/or CCR2 on Ly6C^{high} splenocytes are increased by at least about 60%, 70%, 80%, 90%, or 100%, e.g., doubled.

Furthermore, expression of Hsp40 of Dusp1 by Ly6C^{high} splenocytes can be measured. In some embodiments, a therapeutically effective amount of an oral or mucosal anti-CD3 antibody composition is the amount necessary increases the expression levels of Hsp40 of Dusp1 by Ly6C^{high} splenocytes by about 20% or more. In some embodiments, levels of expression of Hsp40 of Dusp1 by Ly6C^{high} splenocytes are increased by at least about 60%, 70%, 80%, 90%, or 100%, e.g., doubled.

The methods of treatment or prevention typically include administering to a subject an oral or mucosal anti-CD-3 antibody composition sufficient to stimulate the mucosal immune system. In some embodiments, the methods include administering an oral or mucosal anti-CD3 antibody composition sufficient to increase IL-10 and/or TGF-$production by T cells in the peripheral blood, e.g., regulatory T cells, e.g., by about 100%, 200%, 300% or more. In some embodiments, the methods include administering an oral anti-CD3 antibody composition sufficient to decrease T cell proliferation in the peripheral blood, e.g., by about 20%; e.g., in some embodiments, by at least about 20%, 30%, 40%, 50%, 60%, 70%, 80%, 90%, or more.

In some embodiments, the present methods result in a reduction in the levels of IL-6, IL-1ß, IFN-γ, and/or IL-18 in the subject of at least 10%, at least 20%, at least 30%, at least 40%, at least 50%, at least 60%, at least 70%, at least 80%, at least 90%, or at least 95% compared to the levels prior to the administration of the anti-CD3 antibody. In some embodiments, the present methods result in a reduction in the levels of IL-6, IL-1ß, IFN-γ, and/or IL-18 in the subject of 5-10%, 10-20%, 20-30%, 30-40%, 40-50%, 50-60%, 60-70%, 70-80%, 80-90%, 90-95%, or 95-100% compared to the levels prior to the administration of the anti-CD3 antibody. The levels of IL-6, IL-1ß, IFN-γ, and/or IL-18 may be determined using any suitable method known in the art or described herein, including, for example, the O-link assay. In some embodiments, the levels of IL-6, IL-1ß, IFN-γ, and/or IL-18 are determined in the subject's blood. The levels of IL-6, IL-1ß, IFN-γ, and/or IL-18 may be assessed after any suitable time of treatment, for example, after 1 week, after 2 weeks, after 3 weeks, after 4 weeks, after 5 weeks, after 6 weeks, after 7 weeks, after 8 weeks, after 3 months, after 6 months, after 9 months, after 12 months, after 18 months, after 2 years, after 3 years, or after 5 years of treatment. In some embodiment, the reduction in IL-6, IL-1ß, IFN-γ, and/or IL-18 levels persists through a washout period (e.g., a 1-week, 2-week, 3-week, 4-week, 5-week, 6-week, 7-week, 8-week, 9-week, or 12-week washout period).

In some embodiments, the present methods result in an increase in the levels of CD8 naïve cells in the subject of at least 10%, at least 20%, at least 30%, at least 40%, at least 50%, at least 60%, at least 70%, at least 80%, at least 90%, or at least 95% compared to the levels prior to the administration of the anti-CD3 antibody. In some embodiments, the present methods result in an increase in the levels of CD8 naïve cells in the subject of 5-10%, 10-20%, 20-30%, 30-40%, 40-50%, 50-60%, 60-70%, 70-80%, 80-90%, 90-95%, or 95-100% compared to the levels prior to the administration of the anti-CD3 antibody. In some embodiments, the present methods result in an increase in the levels of CD8 naïve cells in the subject of at least 2-fold, at least 3-fold, at least 4-fold, at least 5-fold, at least 6-fold, at least 7-fold, at least 8-fold, at least 9-fold, or at least 10-fold compared to the levels prior to the administration of the anti-CD3 antibody. In some embodiments, the present methods result in an increase in the levels of CD8 naïve cells in the subject of 1.5-2-fold, 2-3-fold, 3-4-fold, 4-5-fold, 5-6-fold, 6-7-fold, 7-8-fold, 8-9-fold, or 9-10-fold compared to the levels prior to the administration of the anti-CD3 antibody. The levels of CD8 naïve cells in a subject may be determined using any suitable method known in the art, including, for example, flow cytometry. In some embodiments, the levels of CD8 naïve cells are determined in the blood of the subject. The levels of CD8 naïve cells may be assessed after any suitable time of treatment, for example, after 1 week, after 2 weeks, after 3 weeks, after 4 weeks, after 5 weeks, after 6 weeks, after 7 weeks, after 8 weeks, after 3 months, after 6 months, after 9 months, after 12 months, after 18 months, after 2 years, after 3 years, or after 5 years of treatment. In some embodiment, the increase in the levels of CD8 naïve cells persists through a washout period (e.g., a 1-week, 2-week, 3-week, 4-week, 5-week, 6-week, 7-week, 8-week, 9-week, or 12-week washout period).

In some embodiments, the present methods result in a decrease in the levels of CD8 effector cells in the subject of at least 10%, at least 20%, at least 30%, at least 40%, at least 50%, at least 60%, at least 70%, at least 80%, at least 90%, or at least 95% compared to the levels prior to the administration of the anti-CD3 antibody. In some embodiments, the present methods result in a decrease in the levels of CD8 effector cells in the subject of 5-10%, 10-20%, 20-30%, 30-40%, 40-50%, 50-60%, 60-70%, 70-80%, 80-90%, 90-95%, or 95-100% compared to the levels prior to the administration of the anti-CD3 antibody. The levels of CD8 effector cells in a subject may be determined using any suitable method known in the art or described herein, including, for example, flow cytometry. In some embodiments, the levels of CD8 effector cells are determined in the blood of the subject. The levels of CD8 effector cells may be assessed after any suitable time of treatment, for example, after 1 week, after 2 weeks, after 3 weeks, after 4 weeks, after 5 weeks, after 6 weeks, after 7 weeks, after 8 weeks, after 3 months, after 6 months, after 9 months, after 12 months, after 18 months, after 2 years, after 3 years, or after 5 years of treatment. In some embodiment, the decrease in the levels of CD8 effector cells persists through a washout period (e.g., a 1-week, 2-week, 3-week, 4-week, 5-week, 6-week, 7-week, 8-week, 9-week, or 12-week washout period).

In some embodiments, the present methods result in an improvement in the Expanded Disability Status Scale (EDSS) score in the subject of at least 10%, at least 20%, at least 30%, at least 40%, at least 50%, at least 60%, at least 70%, at least 80%, at least 90%, or at least 95% compared to the EDSS scores prior to the administration of the anti-CD3 antibody. In some embodiments, the present methods result in an improvement in the Expanded Disability Status Scale (EDSS) score in the subject of 5-10%, 10-20%, 20-30%, 30-40%, 40-50%, 50-60%, 60-70%, 70-80%, 80-90%, 90-95%, or 95-100% compared to compared to the EDSS scores prior to the administration of the anti-CD3 antibody. Methods of determining the EDSS score of a subject are described herein and known in the art (see, e.g., Kurtske; Neurology. 1983 November; 33(11):1444-52, which is incorporated herein in its entirety). The EDSS score in the subject may be assessed after any suitable time of treatment, for example, after 1 week, after 2 weeks, after 3 weeks, after 4 weeks, after 5 weeks, after 6 weeks, after 7 weeks, after 8 weeks, after 3 months, after 6 months, after 9 months, after 12 months, after 18 months, after 2 years, after 3 years, or after 5 years of treatment. In some embodiment, the improvement in EDSS score persists through a washout period (e.g., a 1-week, 2-week, 3-week, 4-week, 5-week, 6-week, 7-week, 8-week, 9-week, or 12-week washout period).

In some embodiments, the present methods result in an improvement in pyramidal scores in the subject of at least 10%, at least 20%, at least 30%, at least 40%, at least 50%, at least 60%, at least 70%, at least 80%, at least 90%, or at least 95% compared to the pyramidal scores prior to the administration of the anti-CD3 antibody. In some embodiments, the present methods result in an improvement in pyramidal scores in the subject of 5-10%, 10-20%, 20-30%, 30-40%, 40-50%, 50-60%, 60-70%, 70-80%, 80-90%, 90-95%, or 95-100% compared to the pyramidal scores prior to the administration of the anti-CD3 antibody. The pyramidal score may be assessed after any suitable time of treatment, for example, after 1 week, after 2 weeks, after 3 weeks, after 4 weeks, after 5 weeks, after 6 weeks, after 7 weeks, after 8 weeks, after 3 months, after 6 months, after 9 months, after 12 months, after 18 months, after 2 years, after 3 years, or after 5 years of treatment. In some embodiment, the improvement in pyramidal score persists through a washout period (e.g., a 1-week, 2-week, 3-week, 4-week, 5-week, 6-week, 7-week, 8-week, 9-week, or 12-week washout period).

In some embodiments, the present methods result in improvement in the ability to walk. The ability to walk may be measured, for example, by the 25-foot timed walk test, where a subject is asked to walk 25 feet as quickly as safely possible. In some embodiments, the present methods result in an improvement in time taken to walk 25 feet in the subject of at least 1 second, at least 2 seconds, at least 3 seconds, at least 4 seconds, at least 5 seconds, at least 6 seconds, at least 7 seconds, at least 8 seconds, at least 9 seconds, at least 10 seconds, at least 11 seconds, at least 12 seconds, at least 13 seconds, at least 14 seconds, at least 15 seconds, at least 16 seconds, at least 17 seconds, at least 18 seconds, at least 19 seconds, at least 20 seconds, at least 21 seconds, at least 22 seconds, at least 23 seconds, at least 24 seconds, at least 25 seconds, at least 26 seconds, at least 27 seconds, at least 28 seconds, at least 29 seconds, or at least 30 seconds compared to the time prior to the administration of the anti-CD3 antibody. In some embodiments, the present methods result in an improvement in time taken to walk 25 feet in the subject 1-5 seconds, 5-10 seconds, 10-15 seconds, 15-20 seconds, 20-25 seconds, 25-30 seconds, 30-35 seconds, or 35-40 seconds compared to the time prior to the administration of the anti-CD3 antibody. The ability to walk may be assessed after any suitable time of treatment, for example, after 1 week, after 2 weeks, after 3 weeks, after 4 weeks, after 5 weeks, after 6 weeks, after 7 weeks, after 8 weeks, after 3 months, after 6 months, after 9 months, after 12 months, after 18 months, after 2 years, after 3 years, or after 5 years of treatment. In some embodiment, the improvement in the ability to walk persists through a washout period (e.g., a 1-week, 2-week, 3-week, 4-week, 5-week, 6-week, 7-week, 8-week, 9-week, or 12-week washout period).

In some embodiments, the present methods result in a stabilization of the subject's EDSS score. In some embodiments, the present methods result in a stabilization of the subject's ability to walk. In some embodiments, the present methods result in a stabilization of the subject's microglial activation. In some embodiments, the present methods result in a stabilization of the subject's levels of IL-6, IL-1ß, IFN-γ, and/or IL-18 levels In some embodiments, the present methods result in a stabilization of the subject's levels of CD8 naïve cells and/or a decrease in CD8 effector cells. “Stabilization” means no substantial increase or decrease (e.g., no increase or decrease of more than 5%) compared to the assessment prior to administration of the anti-CD3 antibody.

In some embodiments, the methods include administering to the subject methylprednisolone sodium succinate 8.0 mg/kg, e.g., intravenously, e.g., 1 to 4 hours before administration of the mucosal anti-CD3 antibody compositions. In some embodiments, the methods can include administering to the subject an anti-inflammatory agent, e.g., acetaminophen or antihistamine, before, concomitantly with, or after administration of the mucosal anti-CD3 compositions.

In some embodiments, the mucosal anti-CD3 antibody compositions are administered concurrently with one or more second therapeutic modalities, e.g., symptomatic treatment, high dose immunosuppressive therapy and/or autologous peripheral blood stem cell transplantation (HSCT). Such methods are known in the art and can include administration of agents useful for treating an autoimmune disorder, e.g., NSAIDs (including selective COX-2 inhibitors); other antibodies, e.g., anti-cytokine antibodies, e.g., antibodies to IFNα, IFNγ, and/or TNFα; gold-containing compounds; heat shock proteins (e.g., as described in U.S. Pat. No. 6,007,821); immunosuppressive drugs (such as corticosteroids, e.g., prednisolone and methyl prednisolone; cyclophosphamide; azathioprine; mycophenolate mofetil (MMF); cyclosporin and tacrolimus; methotrexate; or cotrimoxazole) and therapeutic cell preparations, e.g., subject-specific cell therapy, hematopoietic stem cell therapy. In some embodiments, the methods include administering one or more treatments for multiple sclerosis, e.g., .beta.-interferons (e.g., interferonpla, interferon β1b), mitoxantrone, or glatiramer acetate. In some embodiments, the methods include administering one or more non-anti-CD3 immunosuppressive drugs (such as corticosteroids, e.g., prednisolone and methyl prednisolone; cyclophosphamide; azathioprine; mycophenolate mofetil (MMF); cyclosporin and tacrolimus; methotrexate; or cotrimoxazole) to the subject, e.g., before, during, or after administration of the oral or mucosal anti-CD3 compositions.

### EXAMPLES

### Example 1: Clinical Examination and Pet Imaging of Secondary Progressive MS Patient Treated with Nasal Foralumab

### Patient History

The patient is a 61-year-old man with non-active progressive MS on ocrelizumab. He was first diagnosed with MS at age 41 in March of 2001 when an MRI of the brain and spine that was obtained in evaluation of a month's-long exacerbation of bilateral leg weakness and paresthesias (onset in November 2000) showed lesions that were diagnostically definitive for MS. He reports a history of neurological symptoms with exertion predating that diagnosis by 2 decades. As a high school and collegiate athlete, he noted that strenuous exertion would bring on symptoms of left-lateralized sensory symptoms and horizontal diplopia. After diagnosis, he was treated with injectable MS medications though he tolerated them poorly and had insidious clinical progression despite therapy. He transitioned to rituxan in 2013 and then ocrelizumab November 2018. His MRIs did not show interval change during this span of time, yet gradually his ambulatory status worsened to the point of needing a cane on occasion by early 2016, then more routinely by November 2017 (EDSS of 6, 25-foot walk time of approximately 6 seconds), and now reliant on a wheelchair for distances outside the home, walking substantial slower with a cane and worsening right leg weakness (EDSS 6, 25-foot walk time of approximately 20 seconds) as well as profound imbalance and poor endurance. Consistent physical therapy which helped stabilize his disease early in his disease course has had waning benefit and adjunctive empiric intravenous steroids have failed to stabilize his disease course. His most recent MRIs of the brain and spine in May 2021 were unchanged.

### Treatment Regimen

The patient was treated with nasal Foralumab (anti-CD3) at a dose of 50 μg/day (25 μg/nostril×2 nostrils) three times a week (Monday, Wednesday, Friday) for 2 weeks, followed by a 1-week drug holiday, termed 1 cycle. This cycle was repeated for a total of 5 cycles, or 15 weeks. The patient walks with a cane and has a baseline EDSS score of 6.0.

Foralumab was dosed intra-nasally using either a Controlled Particle Dispersion device (Kurve Technology) or a standard pipette. Dosing occurred in the clinic setting and patient was monitored for one-hour post-dose. Foralumab is a fully human IgG1 anti-CD3 monoclonal antibody. The concentration of the drug administered was 25 μg/100 μl. 100 μl was administered to each nostril×2 nostrils. Total dose administered per day was 50 μg.

### Neurological Evaluation

### Initial Visit

Motor examination: Right pronation and subtle drift and test of pronator drift.

| | Del | Bi | Tri | WE | FE | IO | IP | Quad | Hamst | TA | Gast |
|---|---|---|---|---|---|---|---|---|---|---|---|
| L | 5 | 5 | 5 | 4+ | 4+/5− | 4− | 4− | 5 | 4+/5− | 3 | 5 |
| R | 5 | 5 | 5 | 5 | 4− | 3+/4− | 3+ | 5 | 4 | 3 | 5 |

Sensory examination: Light touch, pinprick and temperature intact. Vibration sensation is absent below the knees bilaterally.

Reflexes: [right/left] Biceps 3+/3+, Triceps 2+/2+, Brachioradialis 3+/3+, Patellar 3+/3+, Ankle jerks 3+/3+, Plantar response is upgoing bilaterally.

### Coordination/Gait:

FNF with moderate ataxia left, sever ataxia right
Wide-based, spastic and ataxic gait. Prominent right leg spasticity.
Romberg test was positive and tandem gait could not be tested due to balance.
Timed 25-foot walk of 18.3 seconds with a cane.
EDSS rated as 6, ambulation index is 4, and disease step is 4.

### 1.5 Month Visit

Motor examination: Normal bulk and tone; no tremor. Subtle right pronator drift.

| | Del | Bi | Tri | WE | FE | IO | IP | Quad | Hamst | TA | Gast |
|---|---|---|---|---|---|---|---|---|---|---|---|
| L | 5 | 5 | 5 | 4+ | 4+ | 4− | 4 | 5 | 4 | 4 | 5 |
| R | 5 | 5 | 5 | 5 | 4+ | 4− | 4 | 5 | 4 | 4 | 5 |

Sensory examination: Light tough, pinprick and temperature intact. Vibration sensation is reduced below the knees on the right; it is mildly decreased on the left toe.

Reflexes: [right/left] Biceps 3+/2+, Triceps 2+/2+, Brachioradialis 3+/3+, Patellar 3+/3+, Ankle jerks 2+/2+, Plantar response is extensor bilaterally.

### Coordination/Gait:

FNF with moderate ataxia bilaterally
Wide-based, spastic and ataxic gait.
Romberg and tandem gait could not be tested.
Timed 25-foot walk of 23.26 seconds with cane. He did not stop during the 25 foot walk.
EDSS rated as 6, ambulation index is 5, and disease step is 4.

### 3 Month Visit

Motor examination: There was no pronation of drift on pronator drift test today.

| | Del | Bi | Tri | WE | FE | IO | IP | Quad | Hamst | TA | Gast |
|---|---|---|---|---|---|---|---|---|---|---|---|
| L | 5 | 5 | 5 | 5−/4+ | 5−/4+ | 5−/4+ | 4 | 5 | 5 | 4 | 5 |
| R | 5 | 5 | 5 | 5−/4+ | 5−/4+ | 5−/4+ | 4 | 5 | 5 | 4 | 5 |

Sensory examination: Light touch, pinprick and temperature intact. Vibration sensation is absent below the knees, at the ankles and distally bilaterally.

Reflexes: [right/left] Biceps 3+/3+, Triceps 2+/2+, Brachioradialis 3+/3+, Patella 3+/3+, Ankle jerks 3+/3+, Plantar response is upgoing bilaterally.

### Coordination/Gait:

FNF and H2S with moderate ataxia bilaterally (left more than right)
Wide-based, spastic and ataxic gait. Prominent right leg spasticity.
Romberg test was positive and tandem gait could not be tested due to balance.
Timed 25-foot walk of 23.6 and 22.9 seconds with a cane.
EDSS rated at 6, ambulation index is 5, and disease step is 4.

Compared to baseline, at 3 months modest improvements in distal upper extremity muscle groups, right iliopsoas, bilateral hamstrings/tibialis anterior. Unchanged walk time and EDSS

### PET Imaging

### PET Radiotracer

[F-18]PBR06 [N-(2,5-dimethoxybenzyl)-2-(18)F-fluoro-N-(2-phenoxyphenyl)acetamide] is a second-generation PET radioligand, targeting the 18-kDa-translocator protein (TSPO), which is overexpressed on activated microglia/macrophages. Strong correlations of [F-18]PBR06-binding with both CD68 expression and TSPO-antibody reactivity have been demonstrated in multiple disease models. [F-18]PBR06 has been studied in healthy human volunteers but not in MS, except for recent studies on white matter and grey matter changes in MS patients.

### Genotyping

Prior to PET scanning, blood samples drawn on the initial screening visit were genotyped for DNA polymorphism of the 18 kiloDalton-translocator protein (TSPO) gene on chromosome 22q13.2, using a TaqMan assay. The index subject was a high-affinity binder.

### Production of Radiopharmaceutical

[F-18]PBR06 was produced in the Nuclear Medicine/Biomedical Imaging Research Core facility at the hospital according to standardized procedures. The product was purified by high-pressure liquid chromatography and sterilized by a 0.22-μm membrane filter. The final product was dispensed in an isotonic solution that was sterile and pyrogen-free for IV administration. The radiochemical purity (RCP) of radiopharmaceuticals was determined using high-pressure liquid chromatography. The organic solvents were determined using gas chromatography. The RCP of the radiopharmaceuticals was >95%.

### PET Acquisition and Analysis.

[F-18]PBR06 was injected as a bolus injection for PET scanning using an IV catheter into the radial antecubital or other arm or hand vein; images were acquired in a list mode acquisition mode using a PET/CT scanner. Standardized uptake value (SUV) images from data obtained between 60-90 minutes post radiotracer injection were reconstructed and interpreted for regional and global radiotracer uptake.

SUV has been shown to correlate with microglial activation in multiple animal models for PBR06 and other TSPO PET ligands. SUVR values are SUV ratios that are a further normalization to a ‘reference’ region in the brain. Because there are no true reference regions in the brain that are truly devoid of any TSPO, such reference regions are referred to as ‘pseudo-reference’ regions in PET literature.

The index patient (EA1) with secondary progressive multiple sclerosis (SPMS) underwent four [F-18]PBR06-PET/CT scans, the first scan was performed prior to starting treatment, and subsequent scans were performed after 3 months of treatment with nasal Foralumab, a subsequent washout period of 7 weeks and a subsequent treatment period of additional 3 months of treatment with nasal Foralumab (i.e., after a total treatment of 6 months with nasal Foralumab). A second SPMS patient (EA2) underwent two [F-18]PBR06-PET/CT scans, one at baseline and the second scan was performed after 3 months of treatment with nasal Foralumab. PET Image Interpretation

### History

A 61 year-old man with SPMS underwent [F-18]PBR06-PET scans before, 3-months after starting treatment with nasal Foralumab and after approximately a 7 week drug holiday following 12 weeks of treatment. PET/CT images were acquired between 60-90 minutes after radiotracer injection. Injected dose was 1.55 mCi and 2.45 mCi for the baseline and follow-up scans, respectively.

### Pre-Foralumab/Baseline PET Scan

Coronal, sagittal and transaxial images of [F-18]PBR06 PET scan reveal widespread, multi-focal, increased radiotracer uptake as compared to background in brain parenchyma, with particularly marked increased radiotracer concentration in bilateral thalami and brainstem. Multiple focal areas of increased radiotracer uptake are also seen in cortical grey matter and juxtacortical white matter.

Mean global brain SUV was 0.78 gram/mL and the mean thalamic SUV was 0.92 gram/mL.

### 3-Months Post-Foralumab PET Scan

Coronal, sagittal and transaxial images of [F-18]PBR06 PET scan reveal diffusely reduced radiotracer uptake in brain parenchyma as compared to the baseline PET scan. Multifocal areas of previously high radiotracer uptake are markedly less prominent and demonstrate reduced confluence. Few faint areas of focal increased radiotracer uptake are seen that have very mildly increased intensity as compared to the brain parenchymal background. Bilateral thalami and brainstem demonstrate significant reduction in PET signal intensity as compared to baseline scan.

Mean global brain SUV was 0.59 gram/mL and the mean thalamic SUV was 0.70 gram/mL, representing a reduction of 24.3% and 23.9% as compared to pre-treatment baseline. This reduction is significantly greater than the test-retest variation seen with [F-18]PBR06 and other similar TSPO PET tracers.

### Impression

There is significant reduction in global and regional brain [F-18]PBR06 uptake, suggesting reduced glial activation following 3-month treatment with nasal Foralumab.

### Post 7 Week Drug Holiday-PET Scan

Coronal, sagittal and transaxial images of [F-18]PBR06 PET scan reveal diffusely reduced radiotracer uptake in brain parenchyma as compared to both the baseline PET scan and the post treatment scan. Importantly, multifocal areas of previously high radiotracer uptake are markedly less prominent and demonstrate reduced confluence. Few faint areas of focal increased radiotracer uptake are seen that have very mildly increased intensity as compared to the brain parenchymal background. Bilateral thalami and brainstem demonstrate significant reduction in PET signal intensity as compared to baseline scan and continued reduction as compared to the post treatment scan.

Mean global brain SUV was 0.56 gram/mL and the mean thalamic SUV was 0.64 gram/mL, representing a reduction of 27.8% and 31.9% as compared to pre-treatment baseline. This reduction is significantly greater than the test-retest variation seen with [F-18]PBR06 and other similar TSPO PET tracers.

### Impression

There is continued and sustained reduction in global and regional brain [F-18]PBR06 uptake, suggesting reduced continued glial activation following a 7 week drug holiday

### 6-Months Post-Foralumab PET Scan

The treatment was well tolerated and there were no symptoms of intolerance or adverse reactions or local irritation in the nasal passage throughout the course of treatment. Importantly, the PET imaging data indicated sustained inhibition of microglial cell activation, which is associated with brain inflammation and cognitive function in MS patients.

TABLE 1
Percent reduction* in microglial PET signal after starting nasal
Foralumab as compared to baseline, in various brain regions

| | WHOLE | CEREBRAL | | WHITE | |
|---|---|---|---|---|---|
| | BRAIN | CORTEX | THALAMUS | MATTER | CEREBELLUM |
| 3 MONTHS | −23% | −23% | −20% | −25% | −22% |
| 7 WEEKS WASHOUT | −27% | −26% | −30% | −29% | −25% |
| 6 MONTHS | −38% | −38% | −50% | −36% | −38% |

*Percent reduction is based on changes from baseline in “SUVR-1”, a surrogate index for PET binding potential. SUVR = Standardized Uptake Value Ratio, calculated with reference to a pseudoreference region in cerebral white matter that showed minimal change in PET SUV, across time points.

Consistent with these clinical and PET observations, the treatment downregulated serum levels of pro-inflammatory cytokines, including interferon-gamma (IFN-γ), interleukin (IL)-18, IL-1p and IL-6, which are known to be associated with multiple sclerosis pathogenesis and progression.

### Clinical Disease Stabilization

Furthermore, clinical disease stabilization was observed as measured by the Expanded Disability Status Scale (EDSS), Timed 25-Foot Walk Test (T25FW), 9-Whole Peg Test (9HPT) and Symbol Digit Modality Test (SDMT). Published PET studies have shown an increase in activated microglial cells in patients with secondary progressive MS (SPMS), an increase associated with higher scores on the Expanded Disability Status Scale (EDSS), a widely-used scale to measure disability.

### 12-Months Post-Foralumab

Nasal Foralumab in this non-active SPMS patient treated over a 12-month period reduced microglial activation on [F-18]PBR06 PET imaging, decreased levels of proinflammatory cytokines, and had positive clinical effects. No side effects were observed.

### Example 2: Randomized, Double Blind Dose Escalation Study of Nasal Foralumab in Heathy Adults

### Methods

### Subjects and Study Design.

The study was a randomized, double blind dose escalation study of 10 μg, 50 μg or 250 μg nasal Foralumab given for 5 days (n=6) or placebo (n=3) at each dose level. Placebo consisted of phosphate acetate buffer. One spray was given into each nostril. There were two sentinel subjects at each dose level (one placebo and one active treatment) to evaluate for serious adverse events. Each subject participated for 30 days. Participants were healthy volunteers, women and men ages 18 to 65 participated. All subjects underwent informed consent and were treated at the Brigham and Women's Hospital's Center for Clinical Investigation (CCI). A controlled particle dispersion device from Kurve Technology® was used for nasal delivery of Foralumab. Patients signed an informed consent form. The study was approved by the Mass General Brigham Human Subjects Research Committee (IRB).

### Study Drug.

Foralumab (28F11-AE; NI-0401) is a fully human IgG1 anti-CD3 mAb with the Fc portion mutated such that the mAb is non FcR binding in vitro which exhibits only minor cytokine release in vivo while maintaining modulation of the CD3/TCR and T cell depletion Foralumab was developed by NovImmune and was acquired by Tiziana Life Sciences.

### Clinical and Laboratory Evaluation.

Subjects underwent clinical (vital signs) and laboratory evaluation (hematology, serum chemistry and urinalysis) for safety and adverse events at days 7, 15 and 30 at which time blood was drawn for immunologic studies. An otolaryngology physical exam including sinonasal endoscopy was performed by an otolaryngologist at the screening visit, visit 5 (final dosing day), and at visit 9 (day 30). A nasal questionnaire was administered at all visits throughout the study.

### In Vitro T Cell Stimulation.

Cell trace labeled healthy donor PBMCs were stimulated in vitro with soluble anti-CD3 Abs (UCHT1 or Foralumab, 2 μg/ml) and rhIL-2 (5 U/ml) or rhIL2 and anti-CD28 (0.5 μg/ml). After 5 days, the cultures were stained for viability, CD4⁺ and CD8⁺.

### Blood Sample Processing.

Subjects gave blood samples at baseline (T1) and at visits scheduled for 7 (T2), 14 (T3), and 28 (T4) days after drug administration. The dates of follow-up varied slightly. Thus, T2 was at 7-10 days, T3 was at 14-18 days, and T4 was at 25-34 days. All blood samples were processed immediately. Plasma was removed by centrifugation of the sodium heparin blood collection tubes after which the blood was then resuspended, diluted with PBS at 1:1 ratio and applied to Ficoll-Hypaque (GE Healthcare) centrifugation to isolate the PBMC buffy coat. PBMCs were counted and resuspended in freezing media (90% FBS/10% DMSO) at 2×10⁷ PBMCs/vial and cryopreserved in liquid nitrogen.

### PBMC Analysis by Flow Cytometry.

PBMCs were thawed at 37° C. into complete RPMI media (with 2% Human AB serum, Gemini Bio), washed with PBS and stained for viability (eFluor 506 viability dye, Invitrogen). 5×10⁶ cells from each sample were subjected to surface stain for lineage and maturation markers followed by staining for intracellular proteins GzmB, Perf, and FoxP3. For surface staining, the cells were resuspended in FcR block (30% in MACS buffer for 15′ at 4C), and then incubated (40′ at 4C) with the panel of surface antibodies that included CD19 (LT19, Miltenyi Biotec), antibodies from Biolegend: CD3 (SK7), CD45RA (HI100), CD127 (A019D5), CD56 (NC1M16.2), CD20 (2H7), and LAPTW4-6H10); antibodies from BD Bioscience: CD4 (SK3), CD8 (SKI), and CD27 (M-T271). After washing with MACs Buffer (0.1% FBS/PBS, 4C), cells were fixed and permeabilized using the eBioscience FoxP3 fixation buffer set, then incubated with permeabilization buffer containing 10% NRS (normal rat serum, 10′ at 4° C.), followed by incubation (30′ at 4° C.) with a panel of intracellular antibodies that included antibodies from Biolegend: Ki67 (K167), FoxP3 (206D), IFNγ (4S.B3), IL-17 (BL168), IL-10 (JES3-9D7) and Perf (dG9), and GzmB (GB11, BD Bioscience). The samples were washed with MACS buffer and each entire sample analyzed on a BD FACS Symphony flow cytometer with HTS attachment.

### T Cell Proliferation Assay.

Upon thawing, 1×10⁷ PBMCs were reserved to generate antigen presenting cells (APCs) after T cell depletion (CD2 beads, Dynal) and irradiation (3200 rads). Total human T cells were isolated from the remaining PBMCs via the human negative Pan T cell isolation kit (Miltenyi Biotec), and then labeled with Cell trace violet (Invitrogen). Cultures were established with 5×10³ Pan T cells/well and 1×10⁴ APCs in a minimum of triplicate wells in 96-well U-bottom plates (Costar) in RPMI-1640 medium (Life Technologies) supplemented with Na Pyruvate, NEAA, HEPES, Glutamine and PennStrep (all from Gibco), and 2% HuS (Gemini Bioproducts). The Tcell/APC cultures were either unstimulated (PBS) or stimulated with Foralumab or commercially available Hit3a or UCHT1 anti-CD3 mAbs from BD Bioscience (no Azide/Low endotoxin) at the indicated concentrations. Some cultures were also supplemented with soluble anti-CD28 (clone 28.2, BD Bioscience, 0.5 μg/ml), rhIL-2 (5 U/ml, Tecileucin**), or TGFβ (Abcam, rhTGFβ, Ab50036). After 5-6 days the cultures were harvested and stained to determine proliferation and expression of cytokines and FoxP3. The cultures were treated with the same PMA/Ionomycin and fixation/permeabilization protocols as in the PBMC assay, but stained with the following antibodies: CD4, FoxP3, IFNγ, IL-17, IL-10, TNFα, PD1, PDL1 TIGIT and LAG3), run on a BD FACS Symphony FACS Analyzer, and analyzed using FlowJo software.

### Single Cell RNAseq.

Immune cells from the participants that received 50 μg Foralumab were analyzed by scRNA-Seq using the 10× Genomics platform. Specific immune populations (CD4⁺ T cells, CD8⁺ T cells, FoxP3⁺ Tregs, B cells, monocytes and dendritic cells), were FACS-sorted from the T1-T4 PBMCs, hash-tagged, and combined to generate specific samples. All samples were submitted and processed through 10× Genomics CellRanger pipeline (v3.0). The analysis of the resultant filtered count matrices was conducted using the Seurat single cell toolkit (v4.1) in R. Count matrices were first demultiplexed and filtered to remove any doublets and negatives. Demultiplexed samples were then filtered further to remove cells with high mitochondrial gene transcript percentages (>20%), cells with low feature diversity (<1000 UMIs), and cells with abnormally high transcript counts (>20000). Data was then normalized and scaled by using Seurat's default parameters with NormalizeData, FindVariableFeatures, and ScaleData functions. PCA was used to reduce the dimensions of the dataset before clustering the cells. Visualization of the clustering was completed through use of the UMAP algorithm packaged within Seurat. Removal of unwanted influence of gender differences was completed using the Harmony package (v0.1.0) before running differential expression analysis within Seurat. Accessory packages for the analysis and visualization of results were dittoSeq (v1.4.4) and ggplot2 (v3.3.5).

### Antigen Arrays.

Antigens were transferred to 384-well polypropylene plates (Genetix, X6004), resuspended in DMSO (1 mg/mL) and spotted onto Epoxy microarray slides (Grace Bio-Labs, 405278) using a microarrayer (Aushon 2470) equipped with solid spotting pins. The microarrays slides were then blocked for 1 h at 37° C. with 1% BSA and incubated for 2 h at 37° C. with a 1:10 dilution of the samples in blocking buffer. The slides were later washed and incubated for 1 h at 37° C. with a 1:100 dilution of goat anti-human IgG Cy3-conjugated and goat anti-human IgM AF647-conjugated detection antibodies (Jackson ImmunoResearch). Blocking, probing, and washing steps were performed using an HS 4800 Pro.

### Hybridization Station (Tecan).

Finally, the slides were scanned using a microarray scanner (Tecan Powerscanner).

### Statistical Analysis.

For comparison of change with time for each of the 57 immunologic markers, each treatment group (10 μg, 50 μg and 250 μg groups and combined placebo patients) were analyzed separately. In each group, the change with time was estimated using a linear mixed effects model with a fixed categorical effect of time and a random intercept. The categorical effect of time allows estimation of the change from the first measurement to each of the subsequent measurements. The random intercept was included to account for the within patient correlation. Subjects with missing measurements were included in this analysis.

### Results

### Demographics and Study Outline:

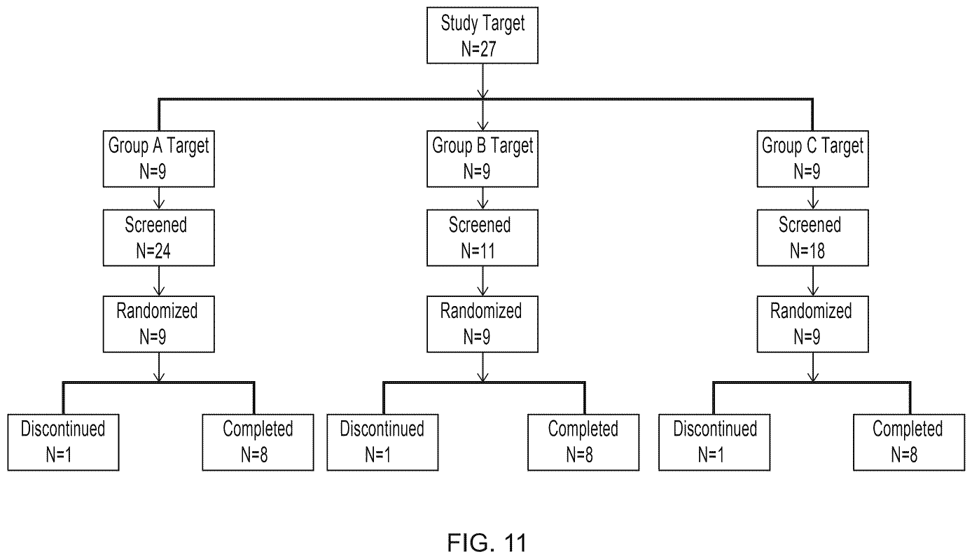

The demographic characteristics of each cohort (10 μg, 50 μg, and 250 μg and the placebo) are shown in Table 2. The patient disposition is shown in FIG. 11. One patient in each dosing cohort discontinued in the study due to non-drug related reasons.

TABLE 2
Demographics of nasal Foralumab dose cohorts (10 μg, 50 μg, 250 μg) and placebo group

| | | | Foralumab | | Foralumab | | Foralumab | | Foralumab - | |
|---|---|---|---|---|---|---|---|---|---|---|
| | Placebo | | (10 μg) | | (50 μg) | | (250 μg) | | all doses | |
| N | 9 | | 6 | | 6 | | 6 | | 18 | |
| Male | 6 | (66.7%) | 4 | (66.7%) | 3 | (50%) | 1 | (16.7%) | 8 | (44.4%) |
| Female | 3 | (33.3%) | 2 | (33.3%) | 3 | (50%) | 5 | (83.3%) | 10 | (55.6%) |

| | | | | | |
|---|---|---|---|---|---|
| Mean age +/− SD | 38.0 +/− 16.6 | 35.8 +/− 15.5 | 24.3 +/− 3.4 | 29.5 +/− 11.0 | 29.9 +/− 11.5 |
| (range) in years | (22-65) | (23-60) | (19-29) | (21-51) | (19-60) |

Race

| | | | | | | | | | | |
|---|---|---|---|---|---|---|---|---|---|---|
| White | 6 | (66.7%) | 3 | (50%) | 2 | (33.3%) | 1 | (16.7%) | 6 | (33.3%) |
| Black or African | 2 | (22.2%) | 0 | (0%) | 0 | (0%) | 1 | (16.7%) | 1 | (5.6%) |
| American | | | | | | | | | | |
| Asian | 1 | (11.1%) | 1 | (16.7%) | 2 | (33.3%) | 2 | (33.3%) | 5 | (27.8%) |
| American Indian or | 0 | (0%) | 0 | (0%) | 0 | (0%) | 0 | (0%) | 0 | (0%) |
| Alaska Native | | | | | | | | | | |
| Native Hawaiian or | 0 | (0%) | 0 | (0%) | 0 | (0%) | 0 | (0%) | 0 | (0%) |
| other Pacific | | | | | | | | | | |
| Other or Unknown | 0 | (0%) | 2 | (33.3%) | 2 | (33.3%) | 2 | (33.3%) | 6 | (33.3%) |
| ethnicity | | | | | | | | | | |
| Hispanic or Latino | 1 | (11.1%) | 1 | (16.7%) | 2 | (33.3%) | 1 | (16.7%) | 4 | (22.2%) |
| Non-hispanic or | 8 | (88.9%) | 3 | (50%) | 1 | (66.7%) | 5 | (83.3%) | 12 | (66.7%) |
| Latino | | | | | | | | | | |
| Unknown or not | 0 | (0%) | 2 | (33.3%) | 0 | (0%) | 0 | (0%) | 2 | (11.1%) |
| reported | | | | | | | | | | |

### Safety.

The treatment was well tolerated by all subjects. No systemic effects were observed at any dose including changes in vital signs (temperature, pulse, blood pressure) or in liver, kidney and hematologic measures (complete blood counts, including differential) during treatment or follow-up. No abnormalities were observed on otolaryngology examination. No EBV reactivation was observed (Table 3).

TABLE 3
Treatment-emergent adverse event (TEAE)

| | | Foralumab | Foralumab | Foralumab | Foralumab - |
|---|---|---|---|---|---|
| | Placebo | (10 μg) | (50 μg) | (250 μg) | all doses |
| System Organ Class | N = 9 | N = 6 | N = 6 | N = 6 | N = 18 |
| Subjects with at least one TEAE | 2 | 5 | 5 | 2 | 12 |
| Number of TEAEs by severity | 3 | 15 | 9 | 3 | 27 |
| Severity | | | | | 27 |
| Non-serious | 3 | 15 | 9 | 3 | 27 |
| Serious | 0 | 0 | 0 | 0 | 0 |
| Ear and labyrinth disorders | | | | | 3 |
| Non-serious | 0 | 2 | 1 | 0 | 3 |
| Serious | 0 | 0 | 0 | 0 | 0 |
| Gastrointestinal disorders | | | | | 2 |
| Non-serious | 0 | 0 | 2 | 0 | 2 |
| Serious | 0 | 0 | 0 | 0 | 0 |
| Immune system disorder | | | | | 1 |
| Non-serious | 0 | 0 | 1 | 0 | 1 |
| Serious | 0 | 0 | 0 | 0 | 0 |
| Infections and Infestations | | | | | 9 |
| Non-serious | 0 | 8 | 0 | 1 | 9 |
| Serious | 0 | 0 | 0 | 0 | 0 |
| Injury, poisoning | | | | | 1 |
| and procedural complications | | | | | |
| Non-serious | 0 | 1 | 0 | 0 | 1 |
| Serious | 0 | 0 | 0 | 0 | 0 |
| Nervous system disorders | | | | | 6 |
| Non-serious | 0 | 3 | 2 | 1 | 6 |
| Serious | 0 | 0 | 0 | 0 | 0 |
| Skin and subcutaneous | | | | | 5 |
| tissue disorder | | | | | |
| Non-serious | 3 | 1 | 3 | 1 | 5 |
| Serious | 0 | 0 | 0 | 0 | 0 |

### In Vitro T Cell Activation by Foralumab.

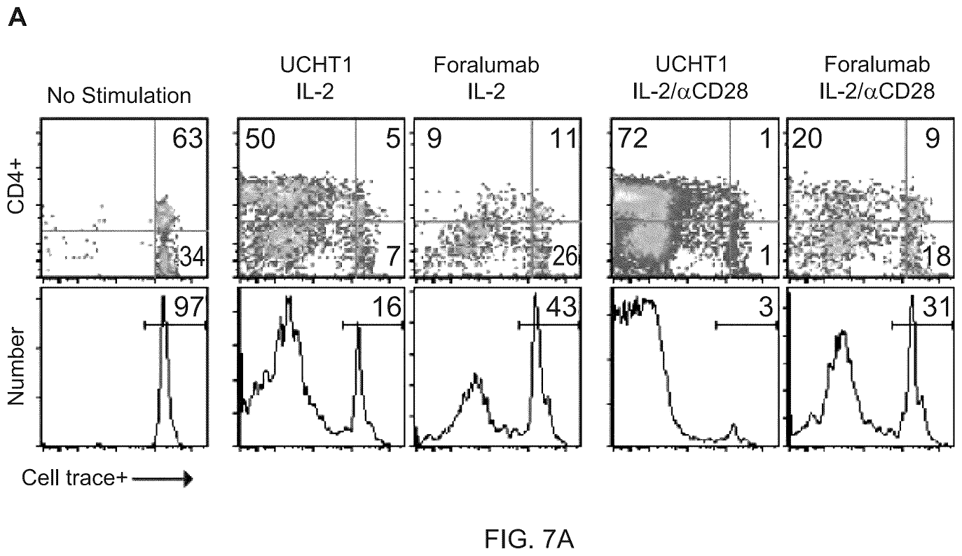

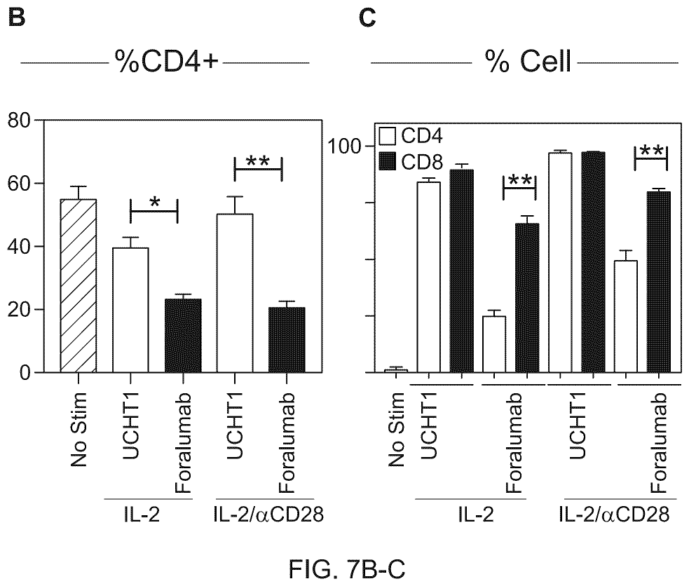

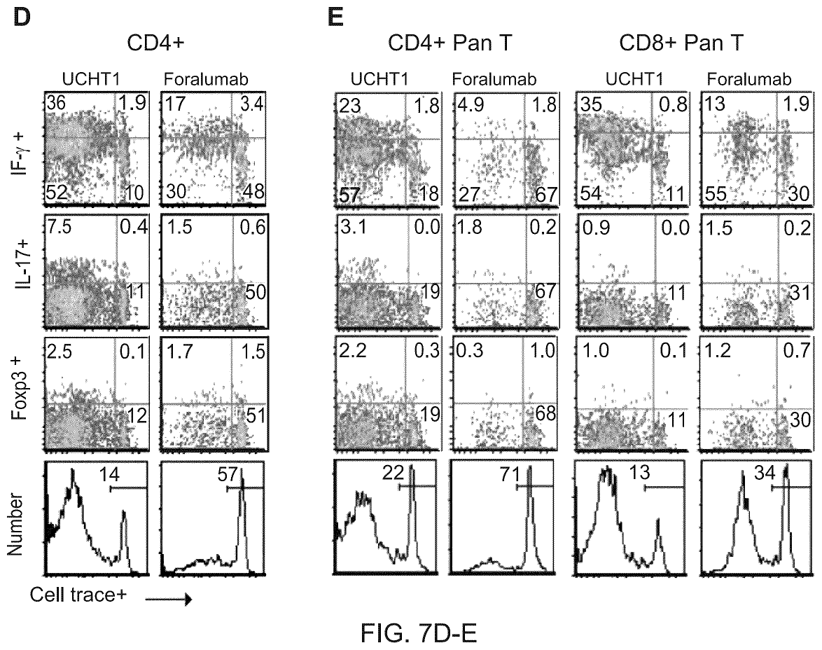

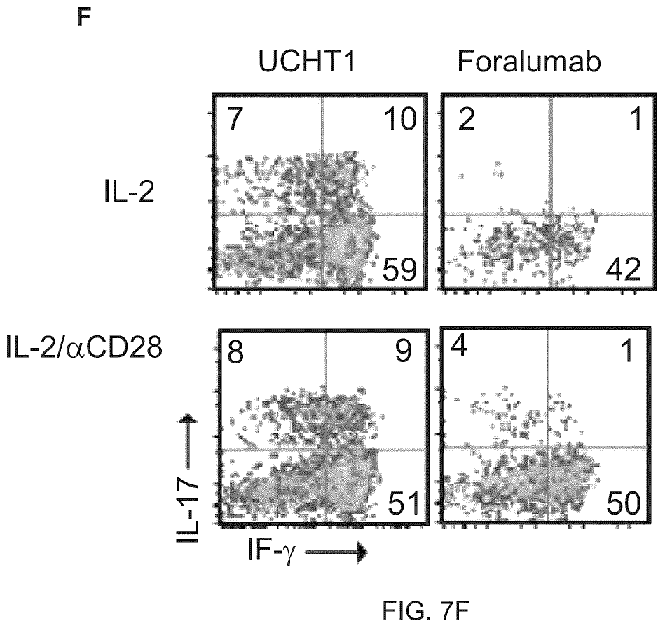

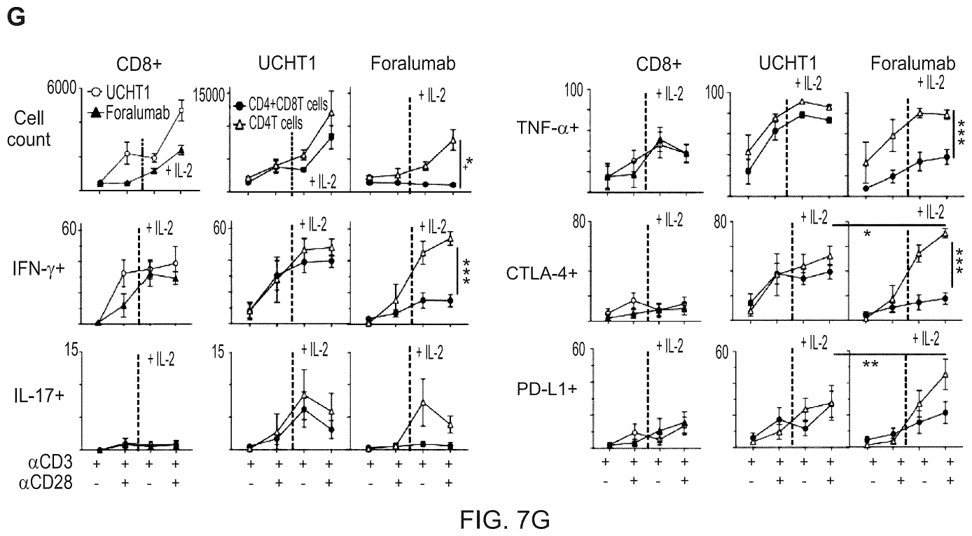

To determine if the fully human, FcR modified, Foralumab antibody induced unbiased human T cell proliferation analogous to that induced by other anti-CD3 antibodies commonly used in research replicate cultures of PBMCs stimulated with either Foralumab (modif IgG1) or the UCHT1 (IgG1) anti-CD3 mAb were established. After 5 days, the cultures were stained to determine viability, CD4⁺ and CD8⁺ lineage and extent of proliferation using cell trace dilution. As shown in FIG. 7, Foralumab induced preferential CD8⁺ T cell proliferation and reduced CD4 T cell proliferation. The histogram plots (FIG. 7A, bottom) show that the mouse anti-human CD3 mAb, UCHT1 induced greater PBMC proliferation than the fully human anti-human CD3 mAb, Foralumab indicated by a more extensive Cell trace dilution and a lower frequency of undivided cells than the Foralumab stimulated cultures (FIG. 7A bottom).

The enhanced proliferative capacity of UCHT1 may be expected as it is fully capable of interacting with FcRs to crosslink and augment TCR signaling. Yet, when the cultures were analyzed to determine the relative expansion of CD4⁺ and CD8⁺ T cells (FIG. 7A top, FIG. 7E, 7F), the different anti-CD3 mAbs induced a striking inequality in CD4⁺ and CD8⁺ T cell expansion where Foralumab selectively expanded CD8⁺ T cells (FIG. 7A-7C). The similar, Foralumab-induced selective expansion of CD8+y T cells occurred in cultures supplemented with IL-2 and IL-2 with anti-CD28, indicating that this was not related to scarcity of co-stimulation. Thus, the mechanism by which Foralumab preferentially activates CD8⁺ T cells remains unclear.

Although identifying an anti-TCR mAb that selectively activates CD8⁺ vs CD4⁺ T cells might appear unusual, the humanized, Fc-altered Tepilizumab anti-CD3 mAb, was also reported to induce selective in vitro expansion of CD8⁺ T cells, which they proposed arose due to Tepilizumab inducing a population of CD8⁺FoxP3⁺ regulatory T cells that killed the CD4⁺ T cells that were present in the same culture. Thus, it was investigated whether Foralumab acted via this mechanism and induced FoxP3⁺ regulatory CD8⁺ T cells that would cause the apparent CD4/CD8 inequality. To test this, it was determined whether CD8 T cells had to be present for Foralumab to induce poor CD4 T cell expansion, and whether the CD8⁺ T cells in Foralumab-stimulated cultures exhibited induction of FoxP3. Thus, T cell cultures of either negatively isolated CD4⁺ T cells only (FIG. 7D), or negatively isolated “Pan T cells” that includes both CD4+ and CD8+ T cells (FIG. 7E, 7F) were established. In addition to providing the different soluble anti-CD3 Abs, the cultures also received irradiated T-cell depleted APCs, and IL-2 (FIG. 7D, 7E) or IL-2/anti-CD28 (FIG. 7F). Here, the frequency of divided CD4 T cells was markedly reduced in cultures that also contained CD8+ T cells (i.e., the stimulation of Pan T cells) as compared to the CD4+ only cultures (FIG. 7D), suggesting that Foralumab induced a CD8⁺ T cell-mediated regulation of CD4+ T cells. However, increase in CD8 expression of FoxP3 in Foralumab-stimulated cultures was observed.

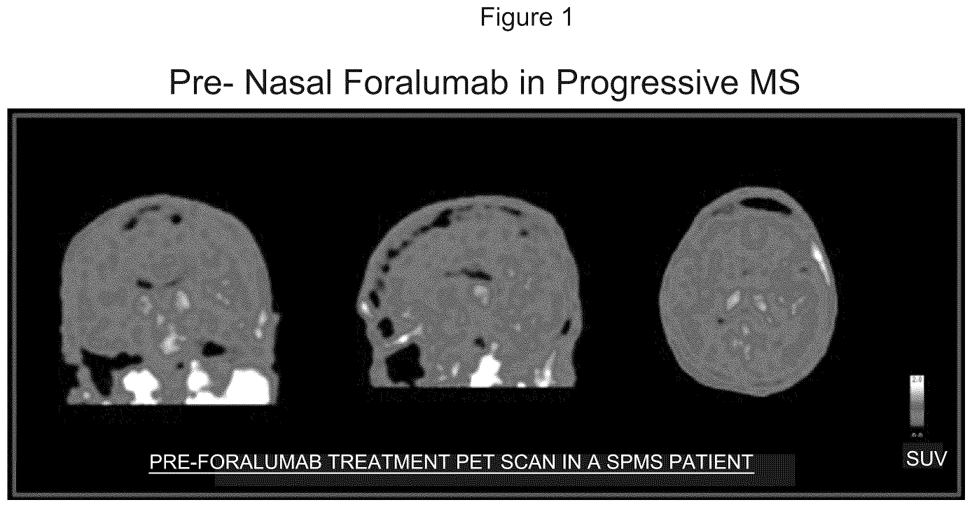

Inhibitory effects of anti-CD3 in humans have been proposed to act by altering the balance of Th subsets. Thus, it was next examined whether Foralumab stimulation resulted in an altered Th1 or Th17 frequency (FIG. 7G). As shown in FIG. 1G, in the presence of CD8⁺ T cells, Foralumab stimulated CD4+ T cells exhibited reduced proliferation and expression of IFNγ (Th1), IL-17 (Th17) and TNF□ as compared to the stimulation of pure CD4⁺ T cells. In the absence of CD8⁺ T cells, Foralumab stimulation of purified CD4⁺ T cells resulted in higher expression of the immune checkpoint molecules, CTLA4 and PDL1 (FIG. 7G), suggesting that Foralumab may also directly induce greater inhibitory molecules on CD4⁺ T cells. Interestingly, the CD8⁺ T cells (from the cultures containing both CD4⁺ and CD8⁺ T cells) showed similar responses to the Foralumab and UCHT1 anti-CD3 antibodies (FIG. 7G). These data suggest that the Foralumab signaling may induce a regulatory CD8⁺ T cell population.

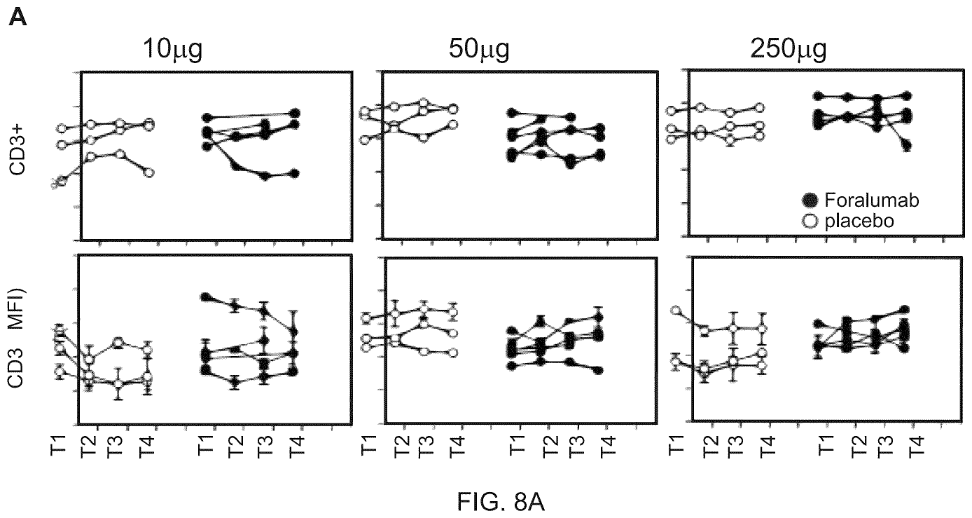

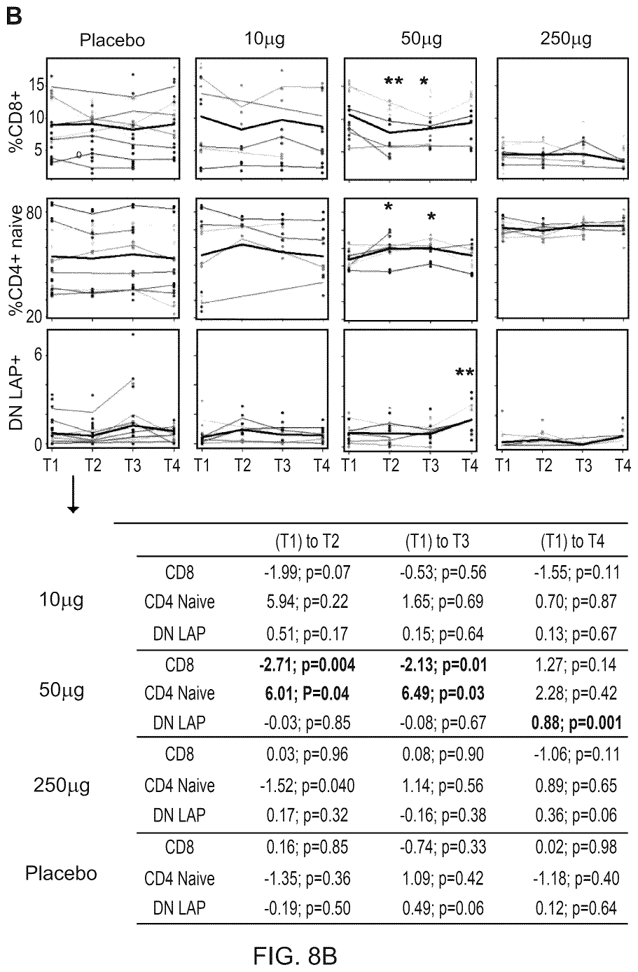

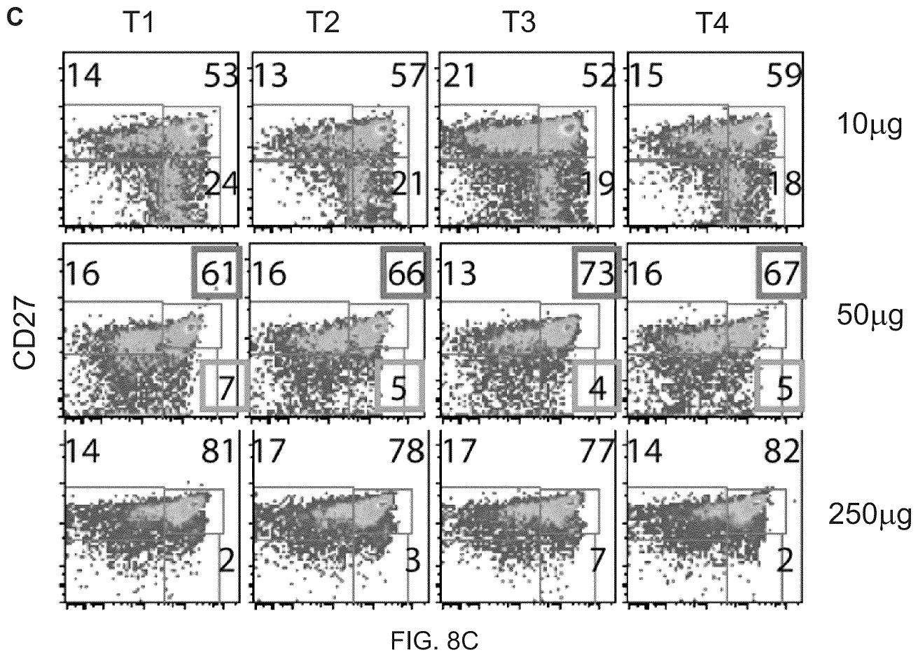

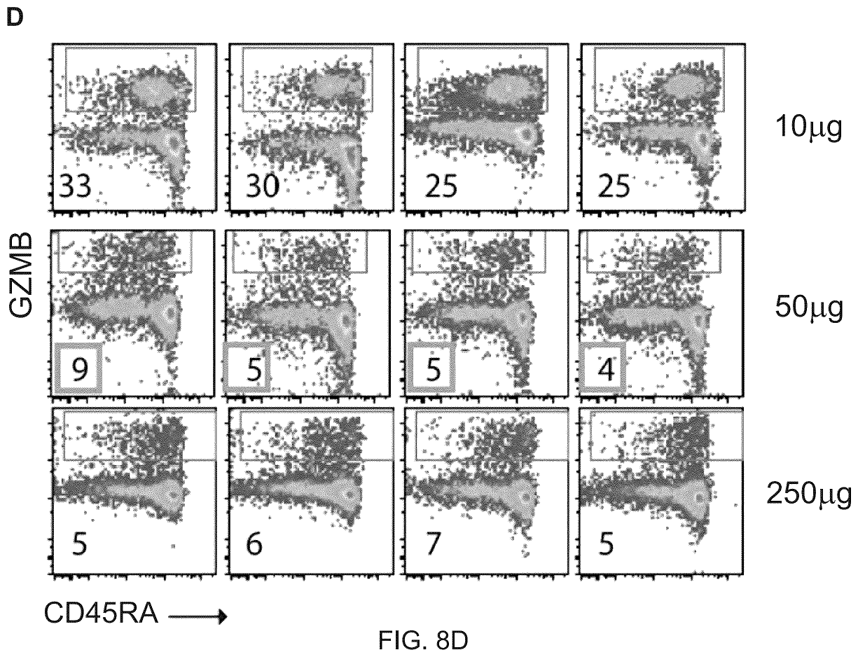

Nasal Foralumab does not modulate CD3 from the T cell surface. IV administration of anti-CD3 mAbs induces the down-modulation of CD3 from the T cell surface. In the study of IV Foralumab in Crohn's disease, CD3 modulation was observed at all dose levels (50 μg, 100 μg, 500 μg and 1000 μg) with the greatest effect seen at the 500 μg and 1000 μg doses. The highest dose of Foralumab administered nasally was 250 μg which is generally less than what has been administered IV with Foralumab and other mAbs. In animal studies, no downregulation of CD3 on T cells was observed following oral or nasal administration of anti-CD3 even at doses that resulted in modulation of CD3 given by the IV route. Whether the lower amounts of Foralumab and the nasal route of administration would result in modulation of cell surface CD3 is unknown. To address this, the longitudinal PBMC samples from baseline (T1) and the T1, T2, and T3 timepoints after the 5-day regimen of daily nasal Foralumab were stained and cytometric analysis for the frequency and mean fluorescence intensity of the cells that bound anti-CD3 was performed. As shown in FIG. 8A, there were no change in the frequency of CD3⁺ cells (top) or the intensity of CD3 expression (MFI, bottom) at any dose in samples obtained beginning at 3 days after the dosing (T2). There also were no change in the frequencies of B cells (as percent of PBMC) or FoxP3⁺ Tregs (as percent of CD4 T cells) or in the CD4 and CD8 ratios over time (not shown).

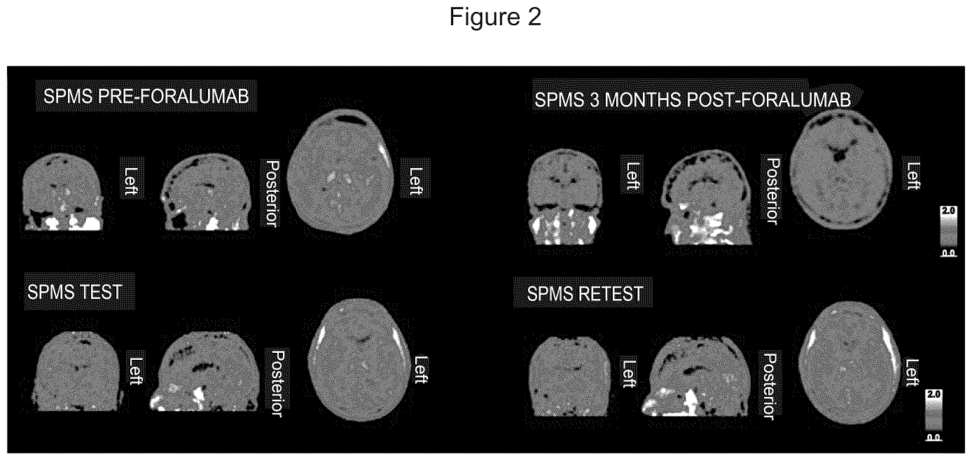

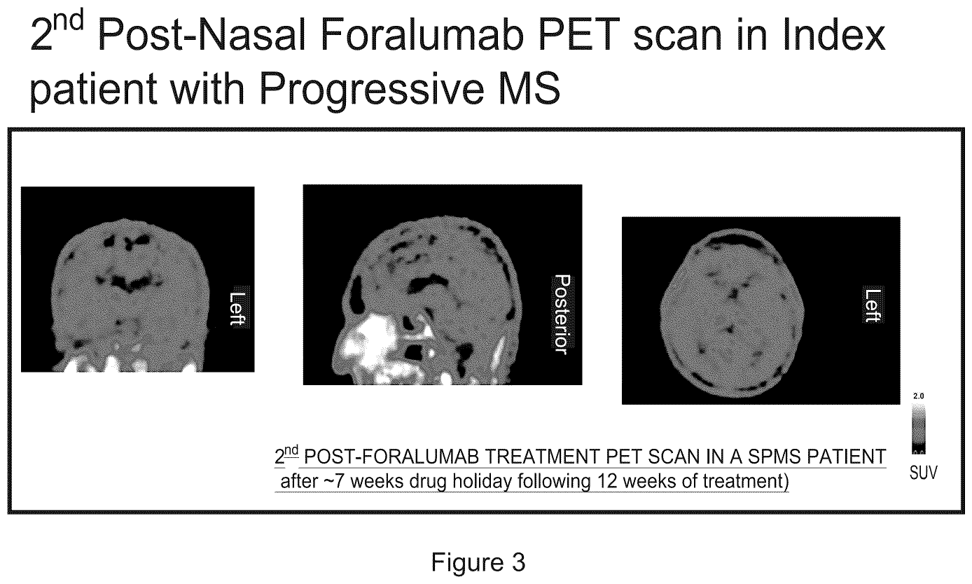

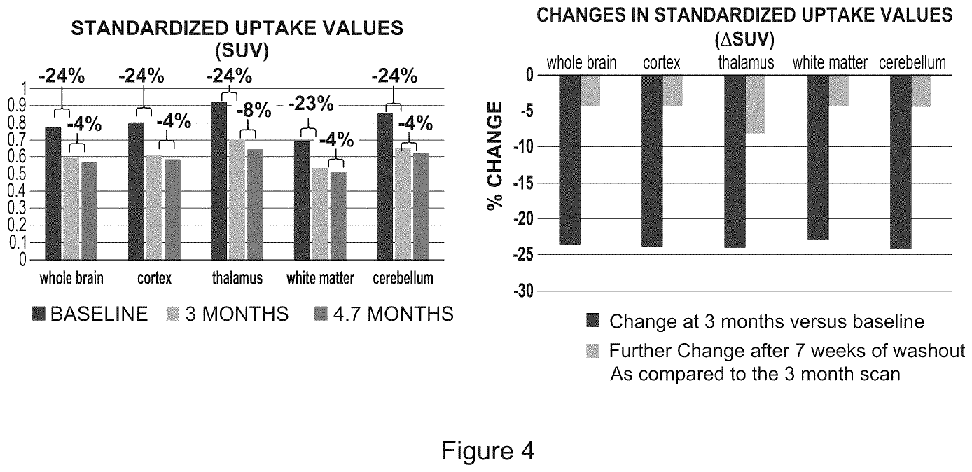

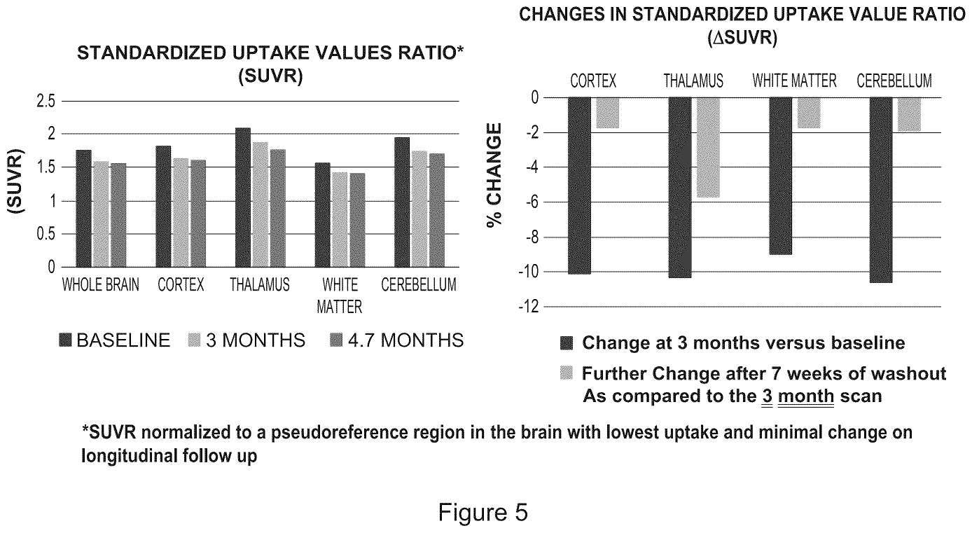

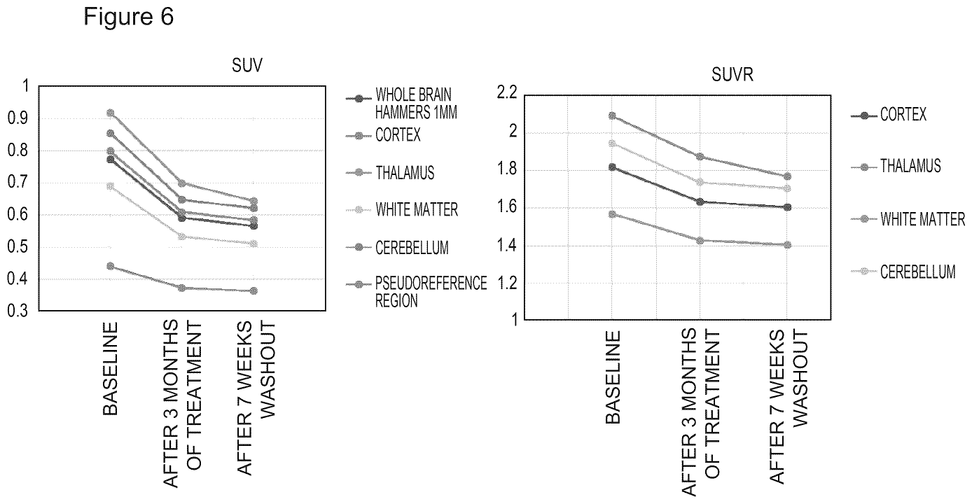

Immune effects of nasal Foralumab occur at the 50 μg dose. To determine whether immunologic effects were observed following nasal Foralumab, PBMCs wee stimulated with PMA/ionomycin for 4 hours and then stained by flow cytometry for surface and intracellular proteins. Pre-treatment (T1) vs the post treatment (T2, T3, and T4) timepoints were compared for the 10 μg, 50 μg, and 250 μg doses and placebo. There were reductions in pro-inflammatory, activated subsets of both CD4 and CD8 T cells that were primarily observed in the group that received the 50 μg dose. CD27 expression was used in lieu of CCR7 to define maturational status as CCR7 expression is reduced on T cells after cryogenic preservation. In terms of CD8+ cells, as shown in FIG. 2B, there was a decrease in the frequency of effector memory cells at T2 and T3. Additional changes observed in CD8⁺ cells in the 50 μg dose included decreased frequency of TEMRAs (CD45RA⁺CD27⁻), increased frequency of naïve cells (CD45RA+CD27+) (FIG. 8C), and decreased expression of granzyme B, though no changes were observed in CD8⁺ central memory cells (FIG. 8D). In terms of CD4⁺ cells, as shown in FIG. 2B there was an increase in CD4⁺ naïve cells at time point 2 and 3. As shown in Table 4 other changes were observed in CD4⁺ cells at the 5 μg dose including a decrease in frequency CD4⁺ effector memory (CD3⁺CD4⁺CD45RA⁻CD27⁻) and TEMRAs (CD3⁺CD4⁺CD45RA⁺CD27⁻) as well as a decrease in granzyme B expression in CD4+ cells. As was the case with CD8⁺ cells, no changes were observed in CD4+ central memory cells. No changes were observed in CD4⁺ Foxp3⁺ cells. A portion of all samples were stimulated in vitro with anti-CD3/IL-2 and there were changes in DN latency associated peptide (LAP)⁺ cells in the patients treated with 50 μg at timepoint 4 (FIG. 8B). No other changes were observed. Of note at the 10 μg dose a decrease in CD8 perforin (T2 and T4) and an increased in CD8 naïve cells (T2) (FIG. 8B) were observed. At the 250 μg dose, there was a decrease in CD4⁺ TEMRA and a decrease of CD4+ granzyme B at T4. However, a decrease in FoxP3⁺ TEMRA, Granzyme and Perform expression was observed at T4 in subjects given the 250 μg dose which is consistent with the observations that immunomodulatory immune effects may be lost at higher doses.

TABLE 4
Estimated change from baseline to each follow-up
time point in patients in the 50 μg dose

| | (T1) to T2 | (T1) to T3 | (T1) to T4 |
|---|---|---|---|
| CD4 FoxP3+ | 0.16; p = 0.61 | −0.01; p = 0.98 | 0.16; p = 0.63 |
| CD8+nv | 8.07; p = 0.003 | 6.75; p = 0.01 | 5.69; p = 0.03 |
| CD8+GzmB+ | −5.43; p = 0.02 | −4.85; p = 0.03 | −5.17; p = 0.03 |
| CD8+Tem | −2.71; p = 0.004 | −2.13; p = 0.01 | −1.27; p = 0.14 |
| CD8+TEMRA | −4.01; p = 0.04 | −3.8; p = 0.06 | −4.4; p = 0.04 |
| CD8+Prf+ | −1.22; p = 0.08 | −1.5; p = 0.04 | −1.38; p = 0.07 |
| CD8+Tcm | −1.44; p = 0.20 | −1.22; p = 0.27 | −0.6; p = 0.61 |
| CD4+nv | 6.01; p = 0.04 | 6.49; p = 0.03 | 2.28; p = 0.42 |
| CD4+Tem | −3.1; p = 0.07 | −3.29; p = 0.05 | −0.66; p = 0.70 |
| CD4+TEMRA | −2.24; p = 0.05 | −2.26; p = 0.05 | −1.81; p = 0.13 |
| CD4+GzmB+ | −2.05; p = 0.06 | −2.25; p = 0.04 | −1.57; p = 0.17 |
| CD4+Tcm | −0.91; p = 0.51 | −0.84; p = 0.54 | 0.03; p = 0.98 |

The estimated difference and p-value were calculated using a mixed effects model with a random intercept. Positive estimated differences indicate that the mean level of the marker increased after administration of the treatment. Bold entries had a p-value less than 0.05.

scRNAseq Analysis in Subjects Receiving the 50 μg Dose.

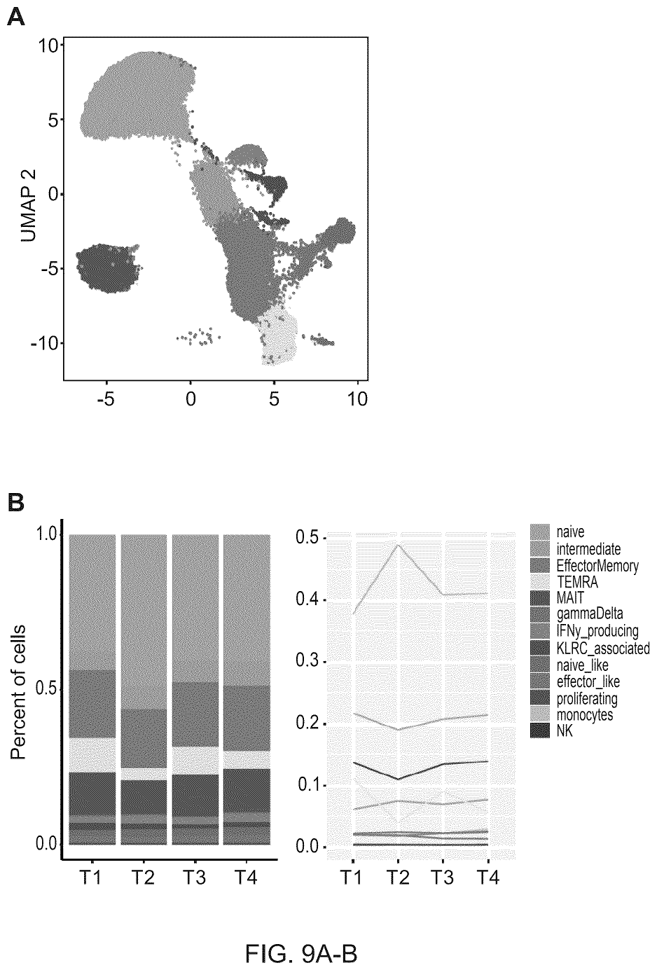

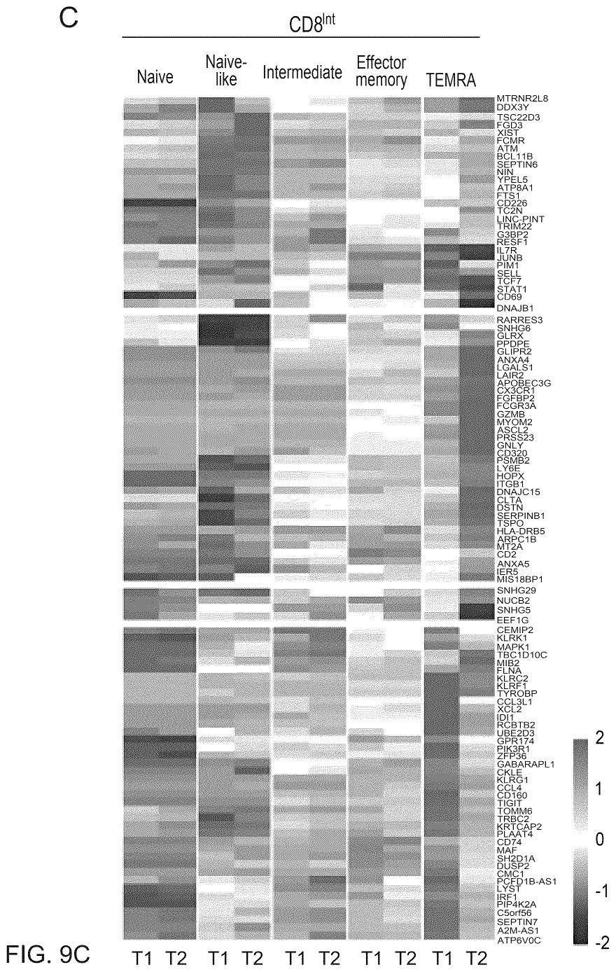

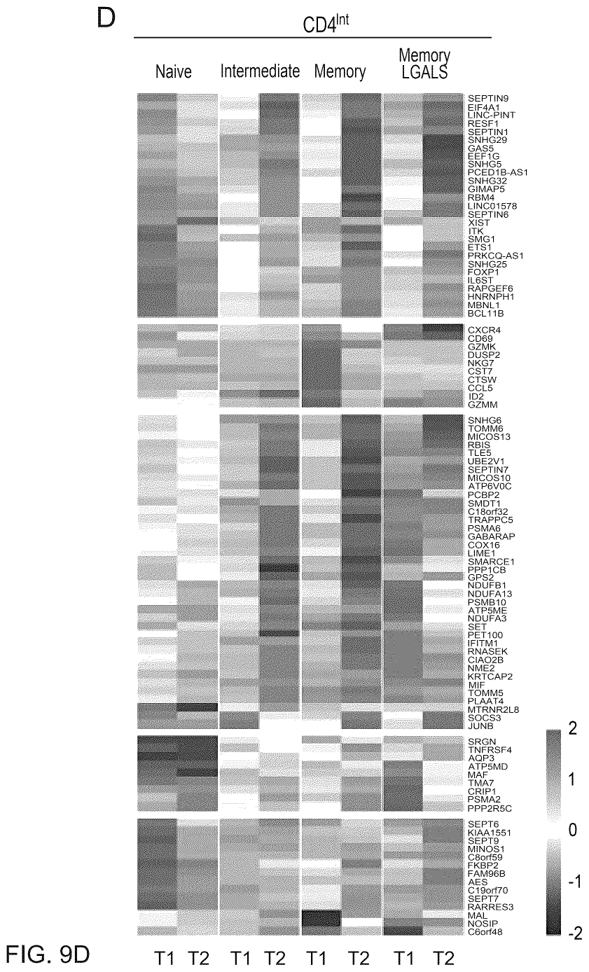

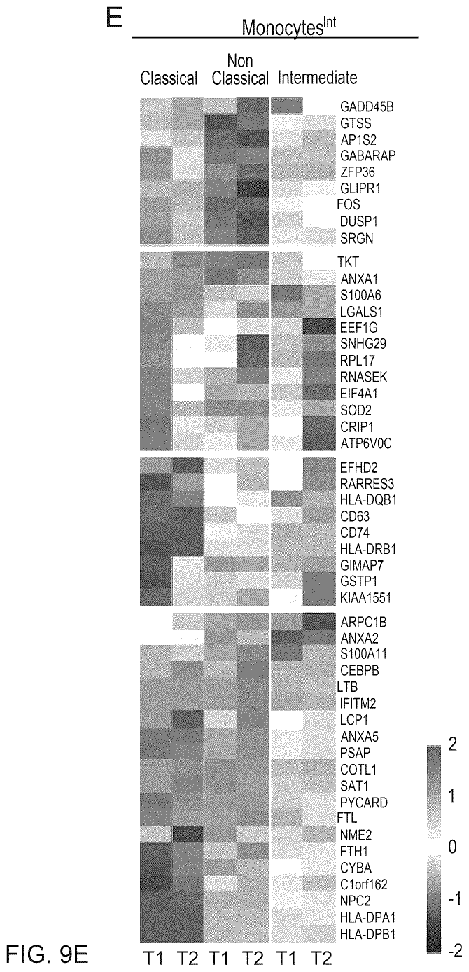

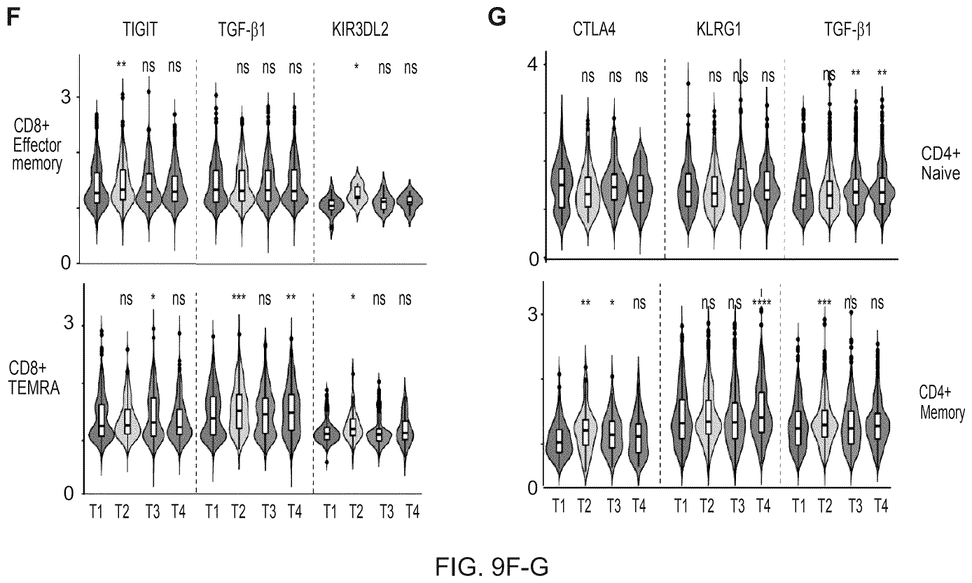

Given that immunologic effects were primarily observed in subjects receiving the 50 μg dose, scRNAseq was performed on isolated immune populations at baseline and post-treatment. Cell populations were FACS-sorted at the same time to prevent batch effects. Consistent with the flow cytometry analysis above, scRNAseq analysis showed a decrease in the frequency CD8 TEMRA and effector memory cells and an increase in the frequency of naïve CD8⁺ T cells (FIG. 9). Most of the changes were observed in the first timepoint after 5 days of treatment (T1, baseline vs T2). Genes that were differentially expressed (DEGs) between baseline (T1) and 3-5 days after treatment (T2) were identified in FACS-sorted CD8%, CD4%, Treg, and monocyte populations (FIGS. 9 C-E). CD8⁺ T cells exhibited the highest DEG (109 genes), with CD4 T cells (non-regulatory), Tregs, and monocytes exhibiting DEG in 94 genes, 5 genes, and 3 genes respectively. Some of the DEG functioned in homeostatic cell biological processes (CD8-28%, CD4-53%, Treg-20%, monocyte-33%), whereas most up- or down-regulated genes have immunologic functions (78 genes in CD8 T cells, 44 genes in CD4 T cells, 4 genes in Tregs, and 2 genes in monocytes).

The function of the immune-related DEG was examined in each cell type to elucidate the immune pathways affected by nasal Foralumab. In the CD8⁺ T cells (FIG. 9C), the genes that were down regulated were primarily involved in promoting survival (STAT1, MTRNR2L8, PIM1, FCMR, and IL7R), augmenting cytokine production (BCL11B, ETS1, TRIM22, TDF7 and JUNB), inducing cell dysfunction (TIGIT, CD160, DUSP2), and enhancing cytotoxicity/signaling (PIP4K2A, PIK3R1, XCL1, FLNA, KLRF1, KLRK, and MAPK1). In contrast, the genes that were upregulated in CD8 T cells were anti-inflammatory as they limit protease/proteosome activity (RARRES3, PSMB2), augment anti-oxidative defense (GLRX, IERS), increase expression of inhibitory receptors (LAIR2, LY6E, and AXNA5), and yet also may promote migration (CX3CR1 and ITGB1).

Next, it was investigated whether the memory CD8⁺ T cell population induced by nasal Foralumab included the induction of TIGIT which are associated with the IV administered anti-CD3 antibody Tepilizumab which has efficacy in treating T1D patients and the induction of certain KIR family member genes which have recently been shown to play a role in regulating autoimmune responses. Indeed, as shown in FIG. 9F, the nasal Foralumab treated CD8+ effector memory populations had induction of TIGIT (T2), and KIR3DL2 (T2) whereas the CD8+ TEMRA population had induction of TIGIT (T3), TGF-B1 (T2, T4) and KIR3DL2 (T2).

The scRNA-Seq analysis of the non-regulatory CD4⁺ T cells indicated that Foralumab treatment resulted in reduced gene expression by all maturational subsets (FIG. 9D). The most affected CD4⁺ T cells were in the activated subsets (intermediate, memory and LGALS1 signature cells) which exhibited reduced expression of genes involved in promoting cell migration (CXCR4, NKG7, CCL5, GzmM and SRGN), cytokine production/signaling (ETS1, IL6ST, BCL11B, JUNB, TNFRSF4) and proteosome activation (PCBP2, PSMA6, PSMB10 and PSMA2). In contrast, the small number of genes that were up-regulated in CD4⁺ T cells appear to function to reduce NF-kB signaling (AES), proteosome activation (RARRES3) and apoptosis (MAL), again indicating a less activated state. Furthermore, as shown in FIG. 9G, in memory CD4⁺ T cells nasal Foralumab induced CTLA4 (T2, T3), KLRG1 (T4), and TGFB1 (T2). These results with CTLA are consistent with changes observed following in vitro stimulation of CD4⁺ T cells by Foralumab (FIG. 7).

The scRNA-Seq analysis of monocytes (FIG. 9E) generated gene based clusters representing three classes of monocytes: 1) classical monocytes, which are associated with anti-bacterial activities; 2) non-classical monocytes, which are involved in immune surveillance, and 3) intermediate monocytes that are the most potent inducers of T cell activation. In the classical monocytes, DEG genes with reduced expression are either induced by inflammation (LGALS1, SOD2, and CRIP1) or promote inflammation (GADD45B, DUSP1, FOS and SRGN). Of the DEG genes in the intermediate monocytes, five were genes that affect antigen presentation (HLA-DQB, HLA-DRB, CD74) or reduce the monocyte activation state (EFHD2 and RARRES3). In the non-classical monocytes, genes involved in antigen presentation (HLA-DPA1 and HLA-DPB1) were increased. It has been reported that DQ and DP restricted T cells produce higher levels of IL-10 whereas DR restricted T cells produce higher levels of IFNγ. Thus, nasal Foralumab induces monocytes that promote a less inflammatory immune response.

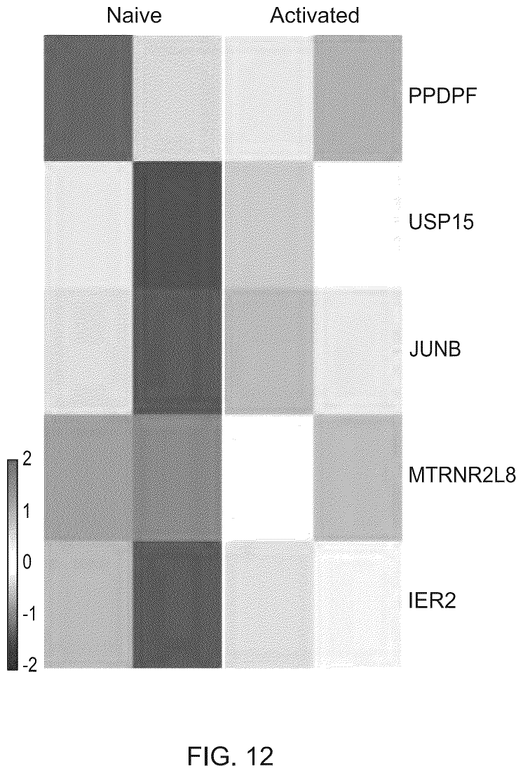

In the Treg population (FIG. 12), only four DEGs were identified, and all had decreased expression. Tregs had reduced expression of JUNB which may enhance Treg stability by inhibiting Th17 differentiation; USP15 which may reduce sensitivity to TGF□ signaling, and MTRNR2L8 which may alter sensitivity to apoptosis.

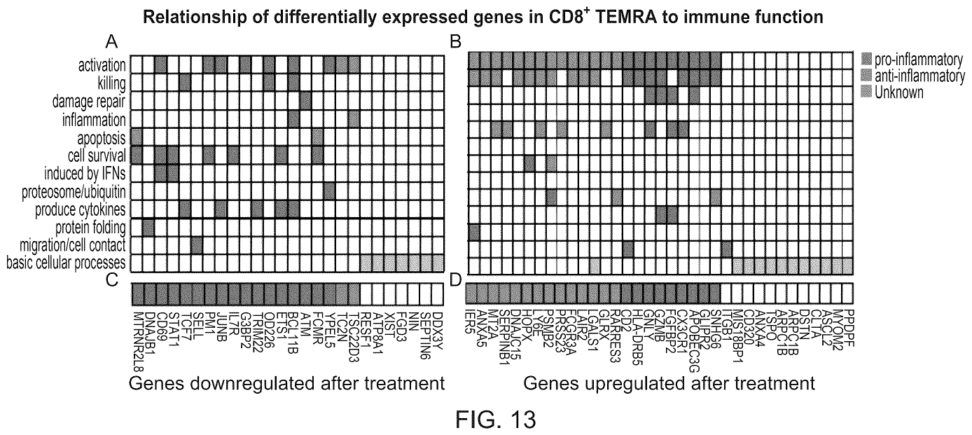

It was then examined the relationship of the differentially expressed genes identified to have immune functions to determine whether the up or downregulated genes tended to be associated with a pro- or anti-inflammatory response. As shown in FIG. 13 for CD8⁺ TEMRA cells, 17/19 genes that promoted inflammatory immune function were down-regulated, whereas 14/24 genes that dampened inflammatory immune function were up-regulated.

### Antigen Microarrays.

Antigen microarrays are a unique tool for the study of the immune system in health and disease. An antigen microarray containing a broad panel of antigens (n=550) that included self and non-self-proteins, heat shock proteins, and infectious agents was used to investigate the effects of nasal Foralumab on the immune repertoire. Previously, antigen arrays had been used to investigate the immune response in healthy subjects treated with oral OKT3 antibody. The effect of nasal Foralumab on IgG and IgM reactivities determined at T1 vs T2 was measured and changes were observed primarily in those receiving the 50 μg dose (FIGS. 10A and 10B). FIG. 10A shows that treatment with nasal Foralumab resulted in significant changes in the reactivity of the T-cell-dependent IgG repertoire. These findings are in agreement with the significant effects on the T cell response detected in functional assays and by scRNAseq.

### Example 3: Treatment with Intranasal Foralumab Showed Positive Clinical Data from a Second Patient with Secondary Progressive Multiple Sclerosis (SPMS)

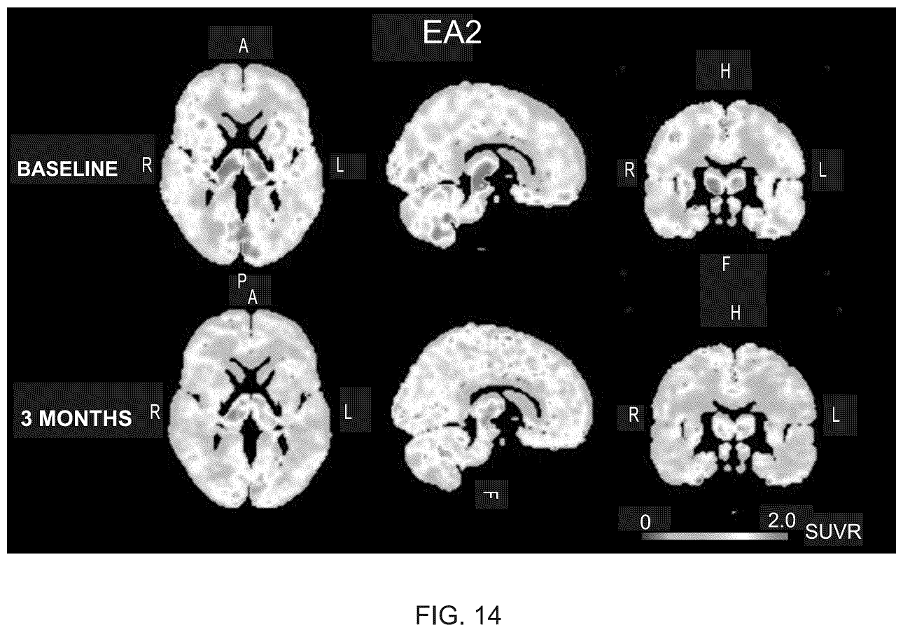

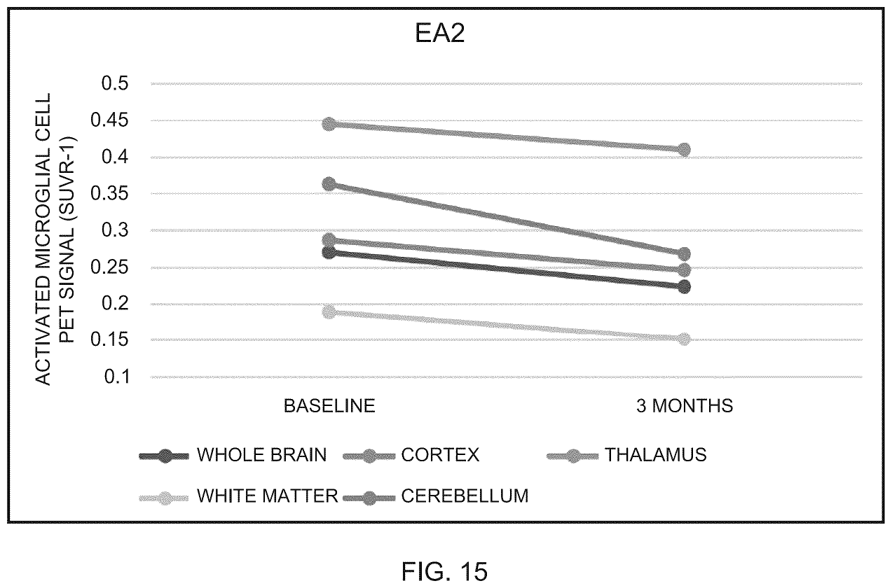

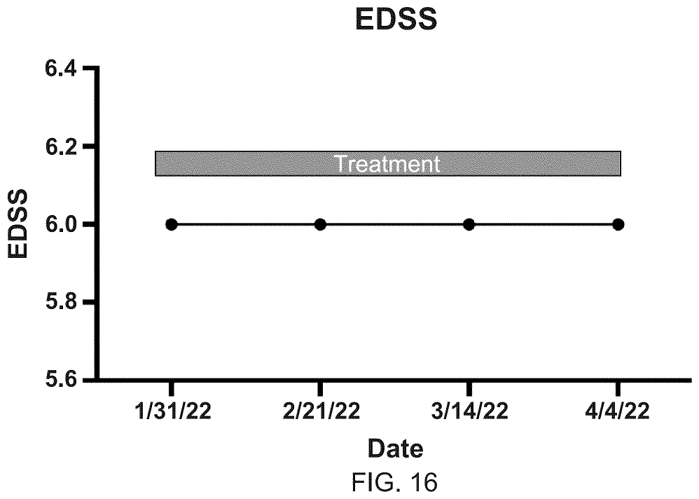

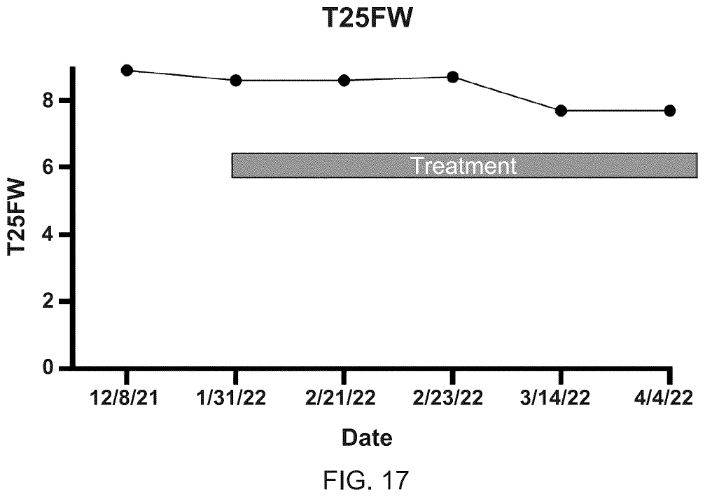

A second patient was treated with nasal Foralumab (anti-CD3) at a dose of 50 μg/day (25 μg/nostril×2 nostrils). The second patient, a young male in his 40s, was diagnosed with SPMS in 2014, and since then, the disease has been progressive, resulting in an accumulation of disability. Following completion of three months of treatment with intranasal Foralumab (three times a week for two weeks, followed by one week off treatment), the patient showed improvement as measured by microglial activation on PET imaging. Approximately 10-30% reduction in PET signal was seen across brain regions (including cortex, thalamus, white matter and cerebellum) in the second SPMS patient (FIGS. 14 and 15), which is comparable to the PET changes seen after three months of treatment in the first SPMS patient treated with intranasal Foralumab (see Table 5). Clinically, the Timed 25-Foot Walk test (FIG. 17) and neurologic exam (FIG. 17) were also improved. Both the first and the second patient are continuing with the treatment and are in their 13th and 4th months of treatment.

TABLE 5
Percent Reduction* in Activated Microglial Cells (AMCs) PET Signal
3 Months After Starting Intranasal Foralumab as Compared to
Baseline, in Whole Brain and Selected Brain Regions in EA2

| | SUVR-1 (% change) | | | | |
|---|---|---|---|---|---|
| | Whole | | | White | |
| | brain | Cortex | Thalamus | Matter | Cerebellum |
| 3 months | −21% | −15% | −9% | −24% | −30% |

*Percent reduction is based on changes from baseline in SUVR-1, a surrogate index for PET binding potential. SUVR = Standardized Uptake Value Ratio, calculated with reference to a pseudo reference region based on prior analysis in EA1.

### Example 4: Clinical Examination and Pet Imaging of Secondary Progressive MS Patient Treated with Nasal Foralumab (Updated Data)

This example describes updated data of the study described in Example 1.

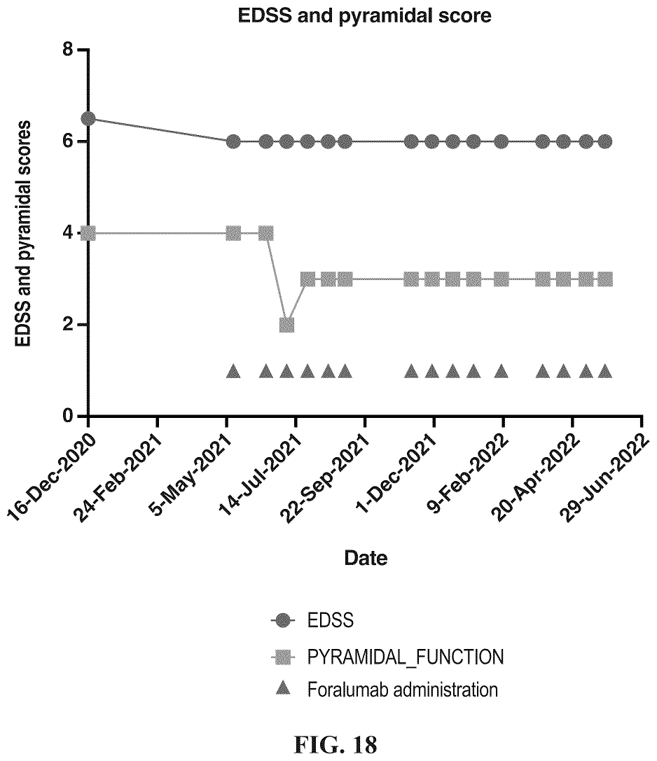

The first patient received a total of 14 Foralumab treatment cycles to date, with two treatment interruptions of about 2 months and about 3 months, respectively) two-month interruption. The patient's EDSS scores were stable to improved over the course of Foralumab administration and the pyramidal scores improved after 3 cycles of Foralumab (FIG. 18).

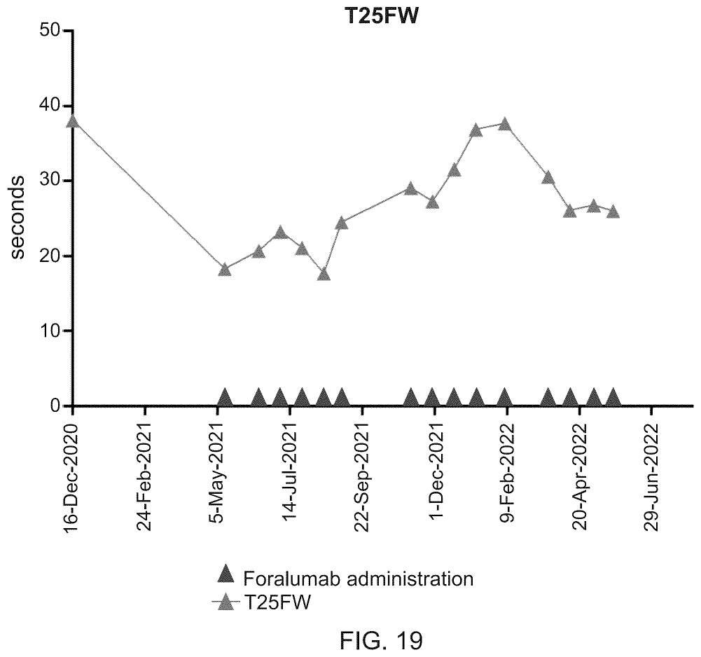

The patient's timed 25-foot walk (T25FW) was stable to improved over the course of Foralumab administration (FIG. 19).

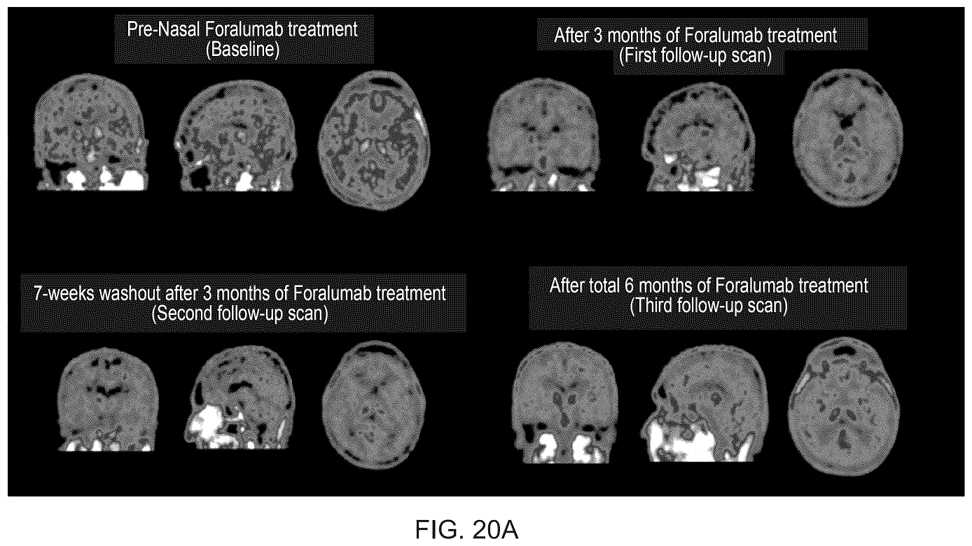

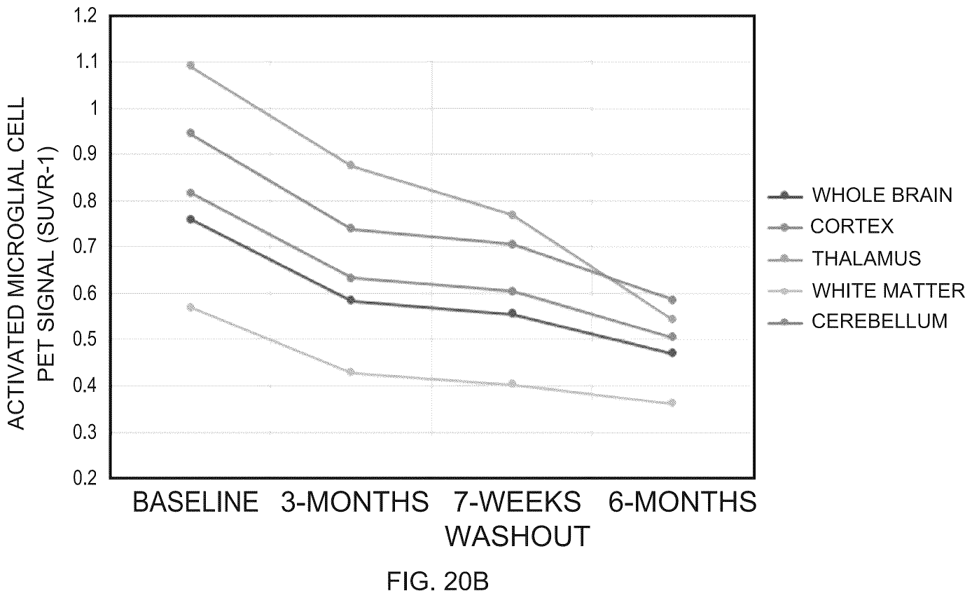

Microglial activation as measured by [F-18]PBR06 PET scan was significantly reduced 3 months after the start of nasal Foralumab, and this reduction was sustained after 7-week washout and at 6 months (FIGS. 20A and 20B).

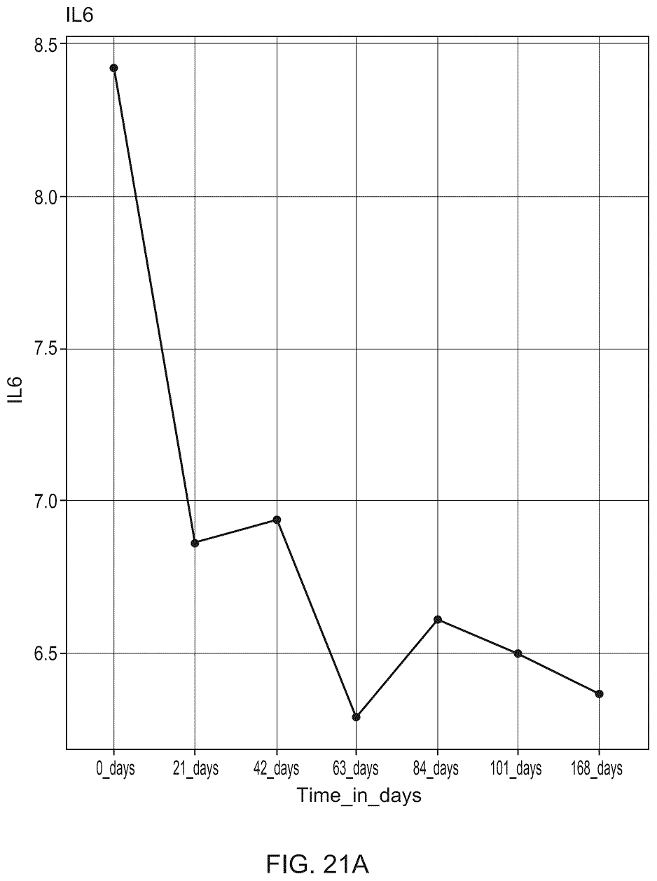

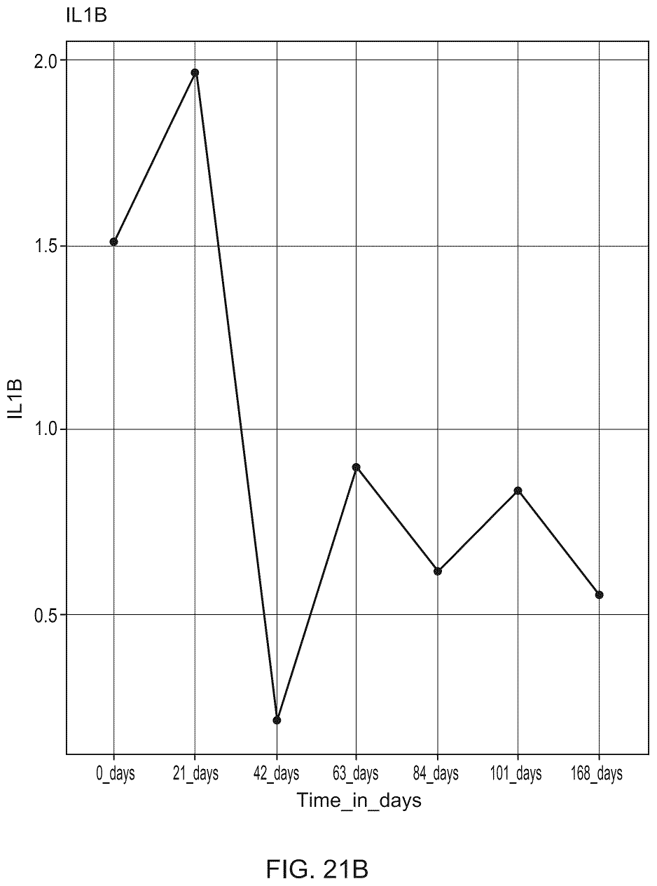

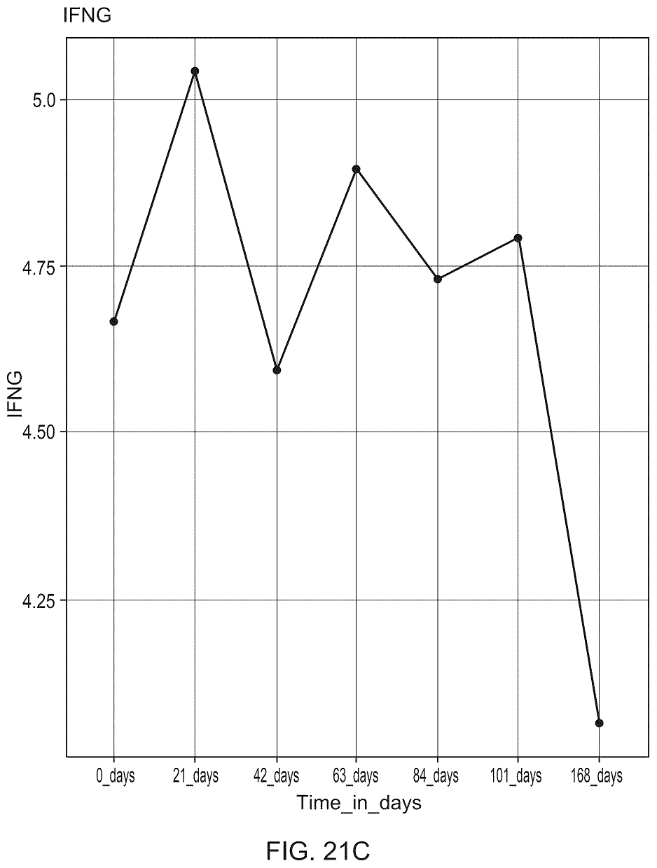

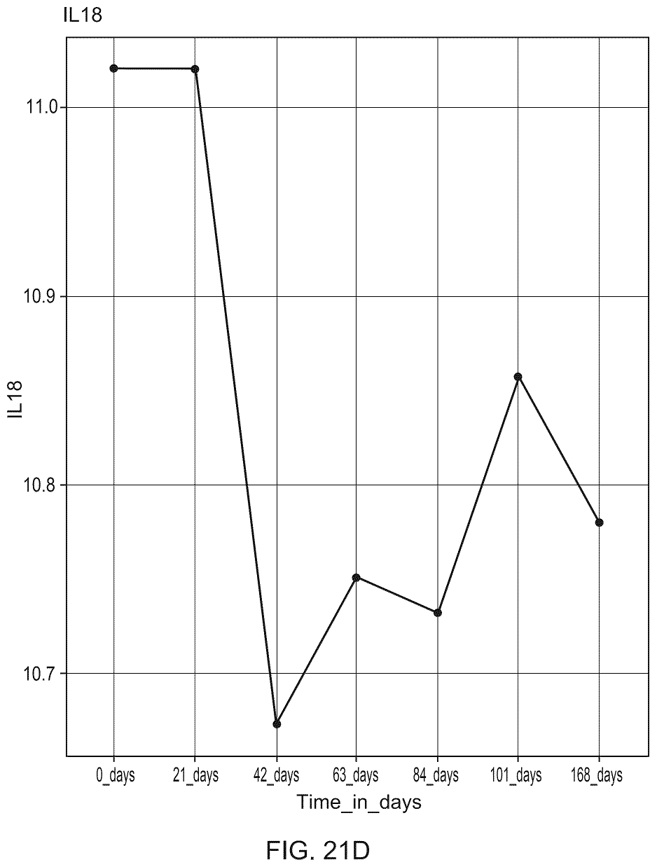

Serum protein measurements of cytokines were performed in batch by the Olink assay showed reduction of IL-6, IL-1ß, IFN-γ, and IL-18 levels (pg/ml) (FIGS. 21A-21D, respectively). Cellular immune studies showed an increase in CD8 naïve cells and a decrease in CD8 effector cells and alteration in gene expression, as measured by single cell RNA sequencing.

### Example 4: Treatment with Intranasal Foralumab Showed Positive Clinical Data from a Second Patient with Secondary Progressive Multiple Sclerosis (Spms) (Updated Data)

This example describes updated data of the study described in Example 3.

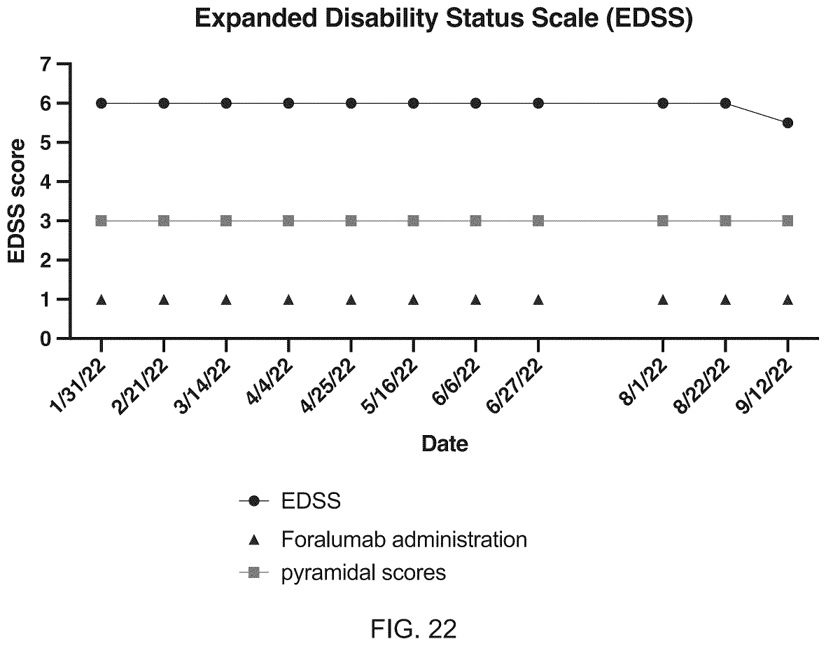

The second patient received a total of 10.5 Foralumab treatment cycles to date, with a treatment interruption of about 11 days. The patient shows improvement in EDSS score on 9/12/22, with a reduction from 6.0 (walking 100 m with a cane) to 5.5, after he demonstrated that he no longer requires a cane to walk 100 m (FIG. 22). The pyramidal score has remained stable (FIG. 22).

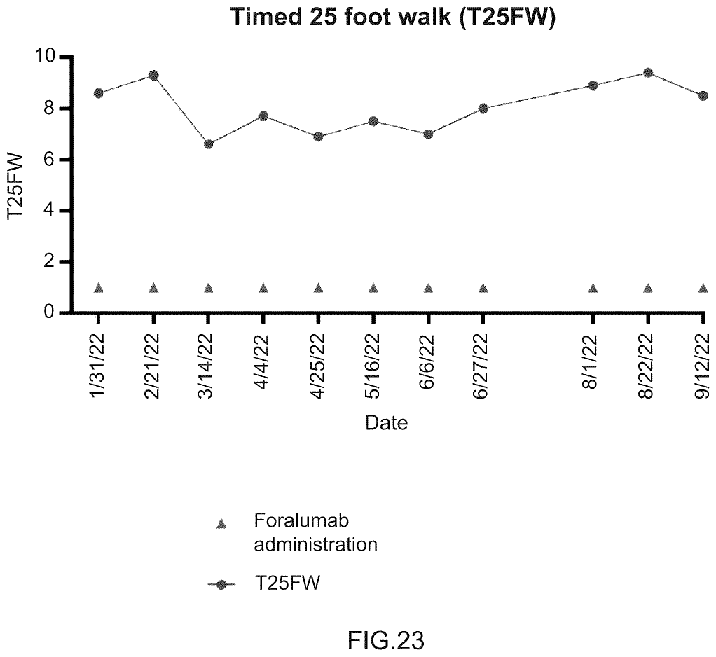

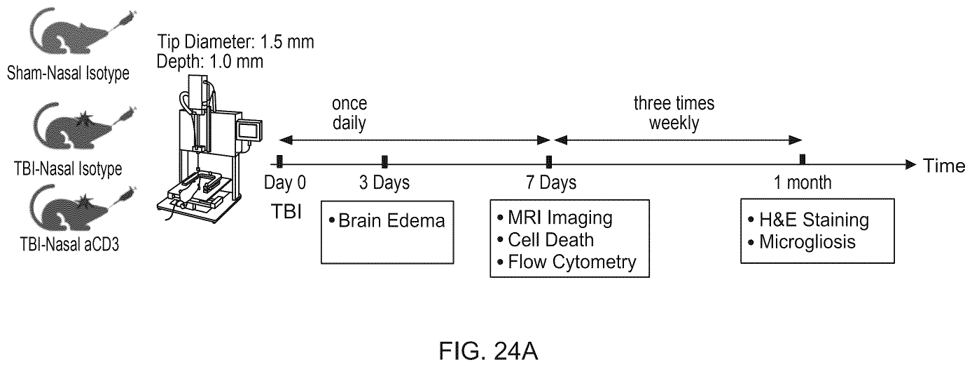

The patient showed improvement in T25FW score on 9/12/22 (FIG. 23). For the two visits prior, he required a cane to walk 25 feet. On 9/12/22, he was able to walk 25 feet without a cane, and his walking time was faster (FIG. 23).

### Example 5: Nasal Anti-CD3 Ameliorates Traumatic Brain Injury by Inducing IL-10 Dependent Tregs that Modulate Microglia Inflammation

### Introduction

Traumatic brain injury (TBI) is a leading cause of death and disability, with both direct and indirect costs (Faul et al., Handb C/in Neurol 127, 3-13 (2015)). TBI is implicated in long-term morbidity, including motor deficits, cognitive decline, and long-term neurodegeneration (Shively et al., Arch Neurol 69, 1245-1251 (2012); Izzy et al. JAMA Netw Open 5, e229478 (2022)). Current treatments have focused on early surgical intervention to limit hematoma expansion and supportive therapy; however, there are few pharmacological interventions to reduce long-term cognitive sequelae post-injury (Langlois, et al, J Head Trauma Rehabil 21, 375-378 (2006); Gordon et al. Am J Phys Med Rehabil 85, 343-382 (2006); McCrory et al. J Athl Train 44, 434-448 (2009); Helmick et al., NeuroRehabilitation 26, 239-255 (2010)). TBI induces a primary mechanical injury followed by a secondary biochemical and cellular response which contributes to neurological impairment (Needham et al. J Neuroimmunol 332, 112-125 (2019)). Neuroinflammation is one of the key mechanisms implicated in both the acute and the chronic pathogenesis of TBI (Algattas et al. Int J Mol Sci 15, 309-341 (2013)). TBI activates resident microglia, induces cytokine release and recruits circulating monocytes and lymphocytes to the CNS, further enhancing inflammation and contributing to secondary injury (Needham et al. J Neuroimmunol 332, 112-125 (2019): Jassam et al., Neuron 95, 1246-1265 (2017)). There is no treatment that targets this neuroinflammatory process, in part because the precise cellular and molecular mechanisms leading to neurological deficits after TBI are largely unknown (Jassam et al., Neuron 95, 1246-1265 (2017). Simon et al., Nat Rev Neurol 13, 171-191 (2017)). Thus, identifying novel therapies that address the chronic CNS inflammation following TBI is a major unmet need.

### Results

### Nasal Administration of Anti-CD3 mAb Ameliorates Neuropathological Outcomes Following TBI.

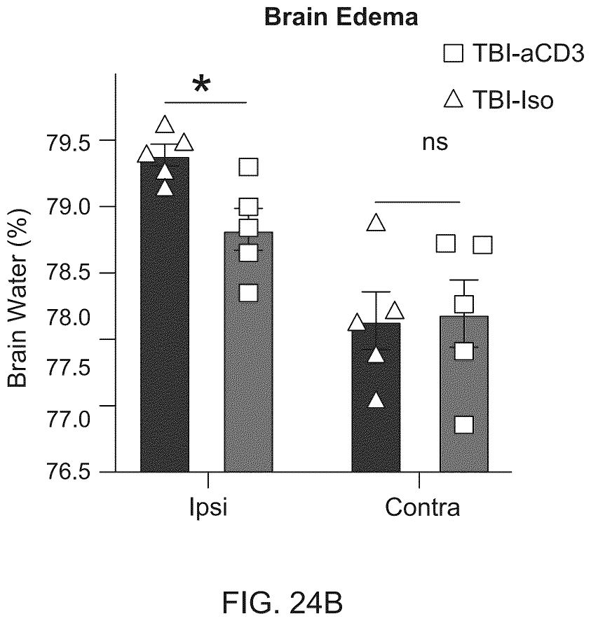

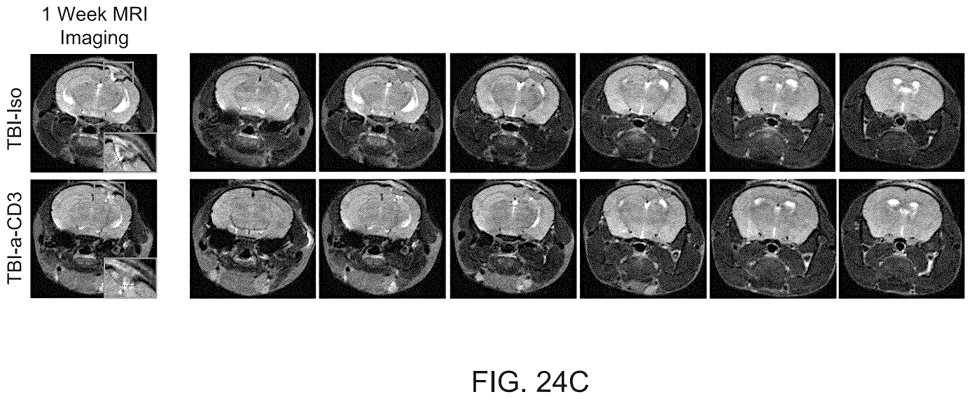

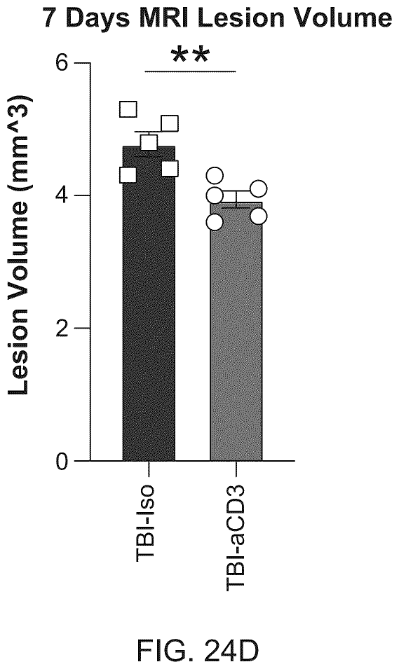

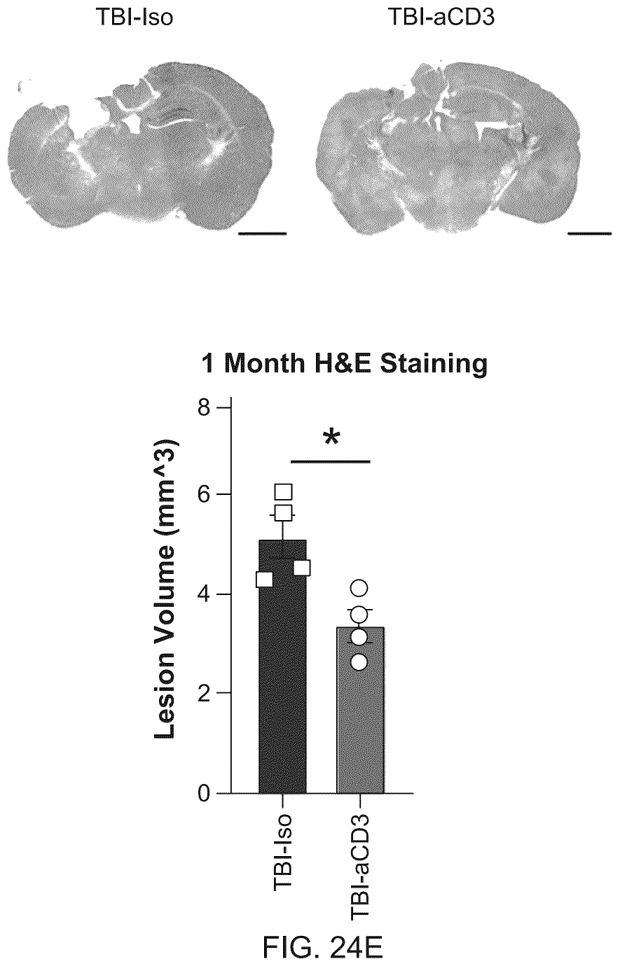

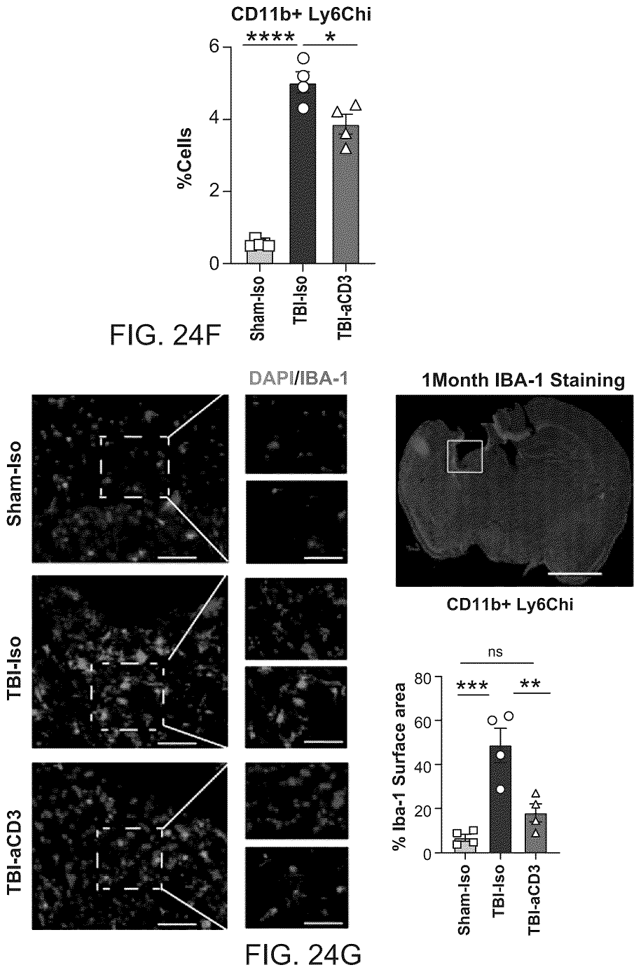

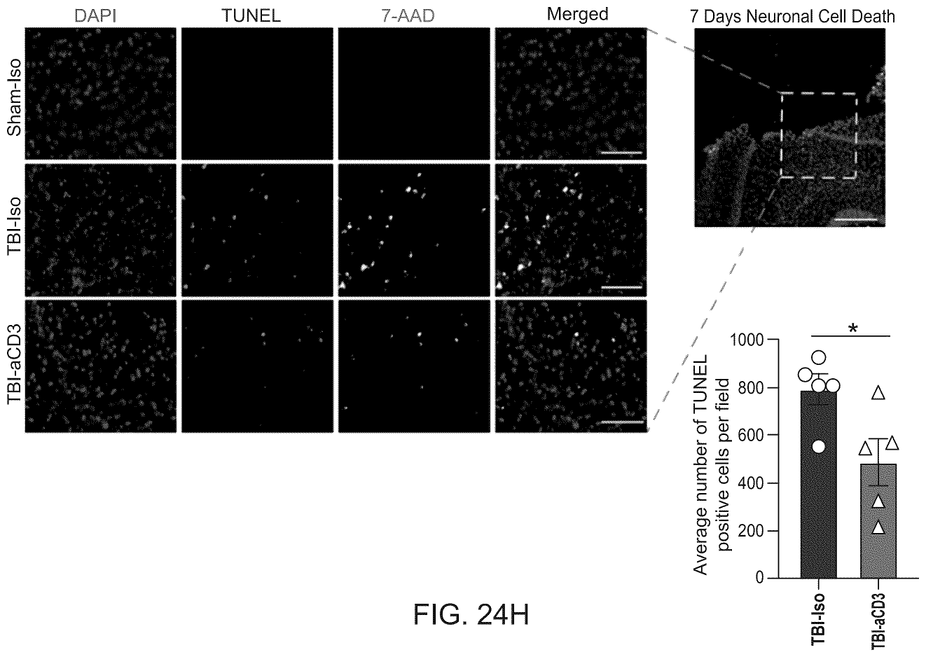

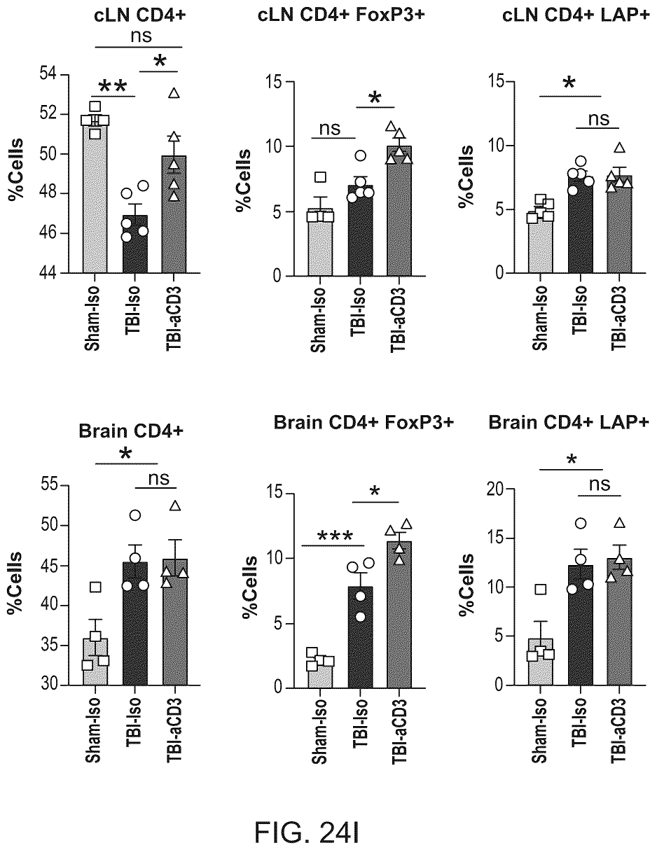

To investigate the therapeutic effect of nasal anti-CD3 in TBI, the CCI model of TBI, which is known for its accuracy and reproducibility (Smith et al. J Neurotrauma 32, 1725-1735 (2015)) was employed to recapitulate moderate to severe TBI features including cerebral contusion, neuroinflammation, BBB dysfunction, and long-term behavioral outcomes. A CCI was induced (1.5 mm tip diameter and 1 mm depth of impact) over the right parietal cortex in C57BL6/J wild-type (WT) mice that were treated with either nasal anti-CD3 (TBI-aCD3) or isotype control (TBI-Iso) starting on the same day of injury, which was continued once daily for 7 days, then 3 times weekly for up to 1-month following injury (FIG. 24A). The percentage of brain edema in the ipsilateral and contralateral hemispheres was assessed at 3 days post-CCI and significant reduction in edema in the ipsilateral hemisphere was found for the TBI-aCD3 group compared to TBI-Iso control (FIG. 24B). The parenchymal lesion volume was examined in Sham-Iso, TBI-aCD3, and TBI-Iso groups at 7 days post-injury, using 3-Tesla Magnetic resonance imaging (MRI) (FIG. 24C). There was significant reduction in ipsilateral lesion volume in in the nasal anti-CD3-treated group compared to TBI-Iso control (FIG. 24D). The lesion volume was also evaluated at 1-month post-CCI using hematoxylin and eosin (H&E) staining and there were significant reduction in ipsilateral lesion volume in TBI-aCD3 mice compared to TBI-Iso control (FIG. 24E).

Consistent with previous reports, CCI was associated with significant increase in monocyte recruitment (CD11b+Ly6c^{hi}) at 5 days post-injury (FIGS. 24F and 24I) and microglia/macrophage activation (Iba-1 staining) at 1-month post-injury compared Sham-Iso control. (FIG. 24G) (Jassam et al. Neuron 95, 1246-1265 (2017); Alam et al. J Neuroinflammation 17, 328 (2020)). CCI also increased cell death, as measured by TUNEL staining at 7 days post brain injury (FIG. 24H). Nasal anti-CD3 treatment reduced monocyte recruitment, microglia/macrophage activation and cell death following CCI(FIGS. 24F-24H). Furthermore, nasal anti-CD3 treatment increased CD4+FoxP3+ Tregs (but not CD4+LAP+) in both cLN and brain following CCI (FIG. 24I), suggesting that anti-CD3-induced Tregs play an important role in restraining inflammation following CCI.

Taken together, these findings demonstrate that nasal anti-CD3 treatment is effective in improving pathological outcomes and treating neuroinflammation and cell death induced in the CCI model of TBI.

### Early Nasal Anti-CD3 mAb Improves Motor and Cognitive Outcomes in Both Moderate-Severe TBI.

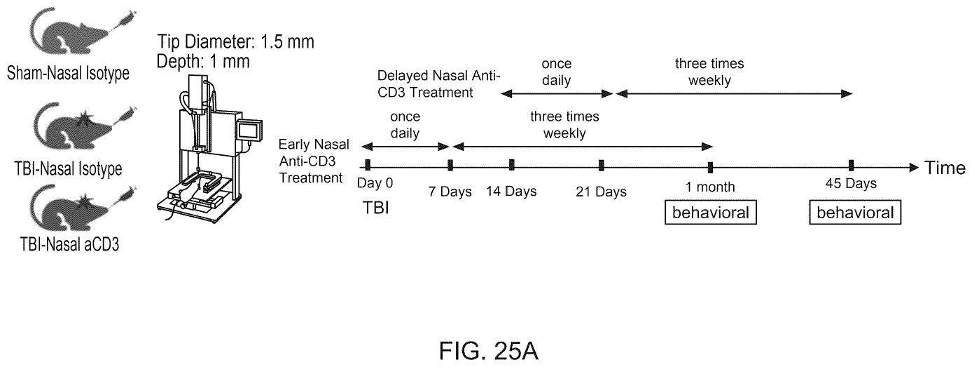

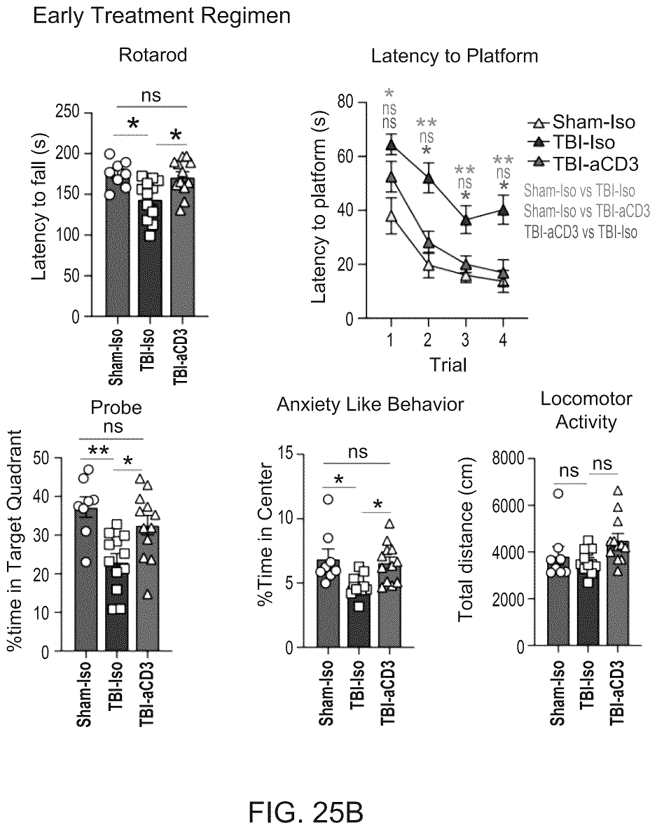

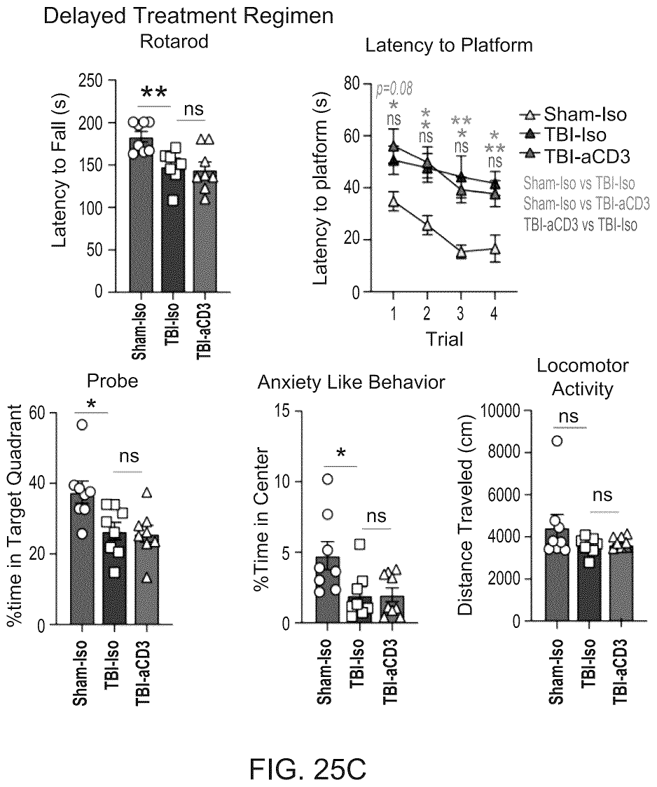

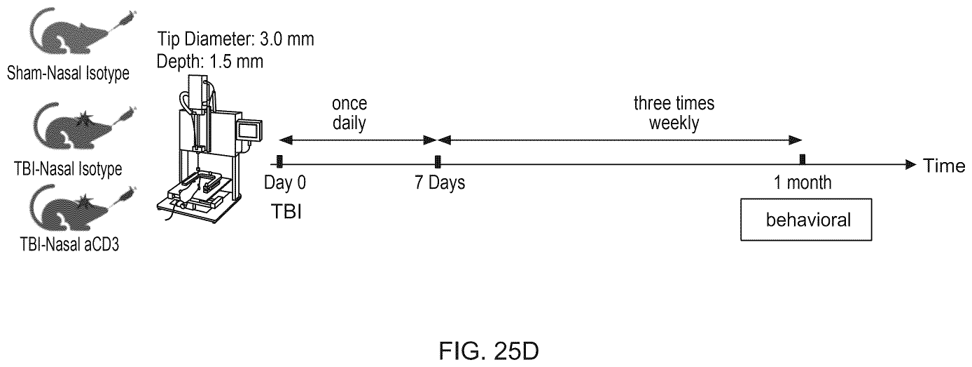

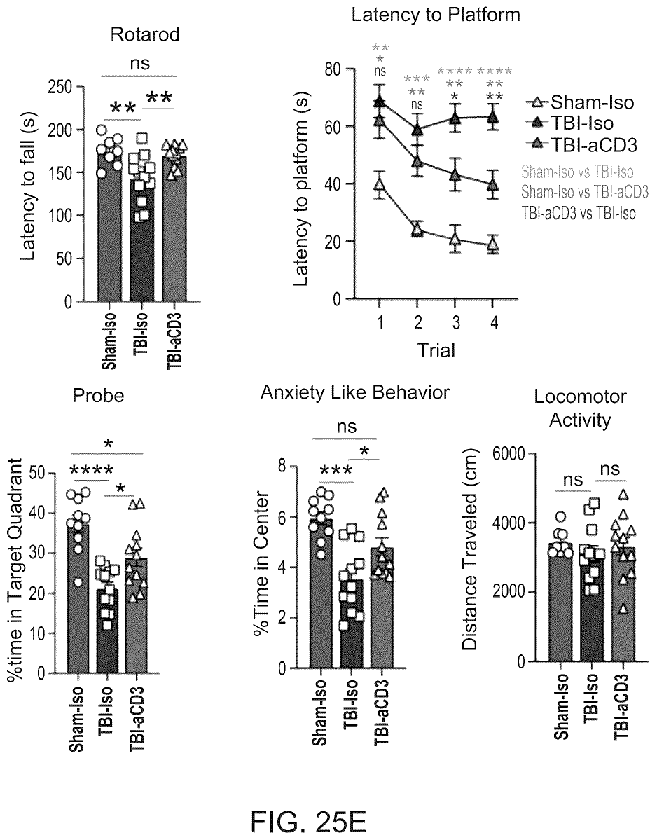

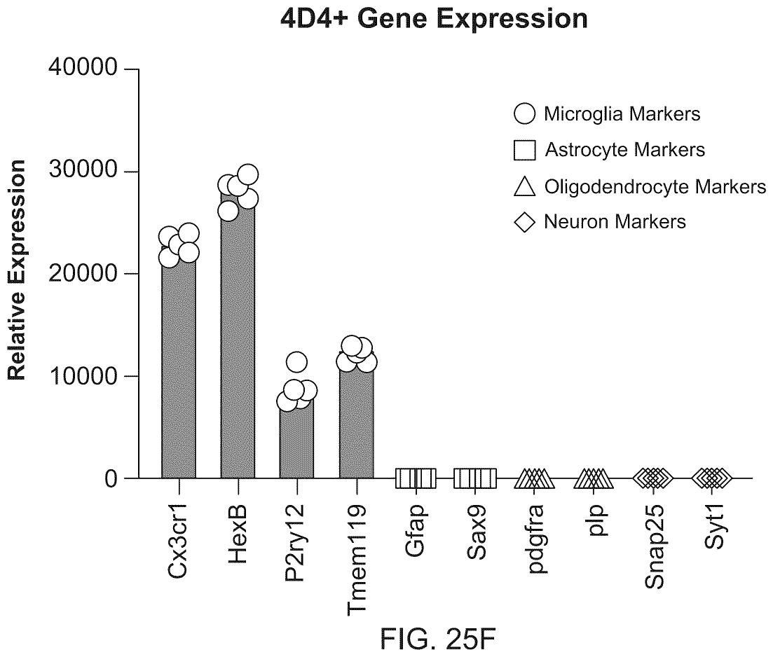

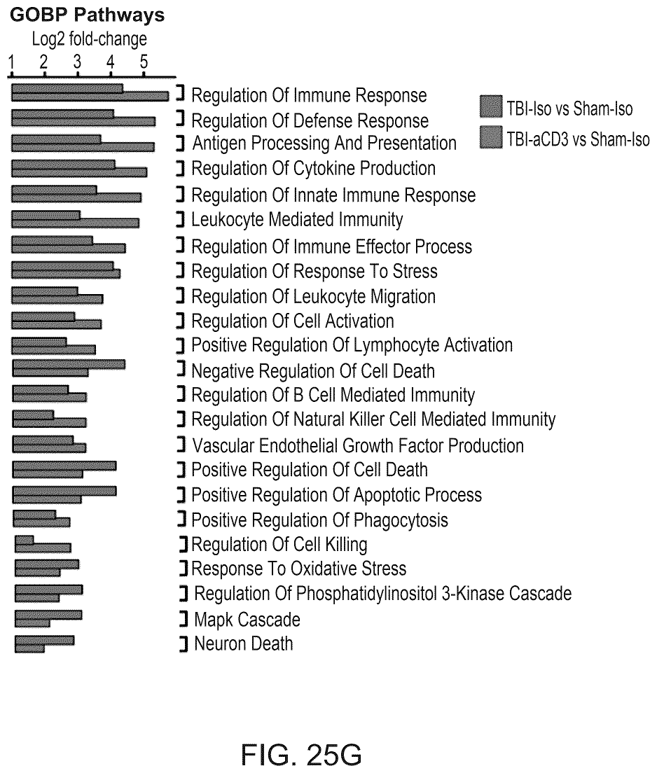

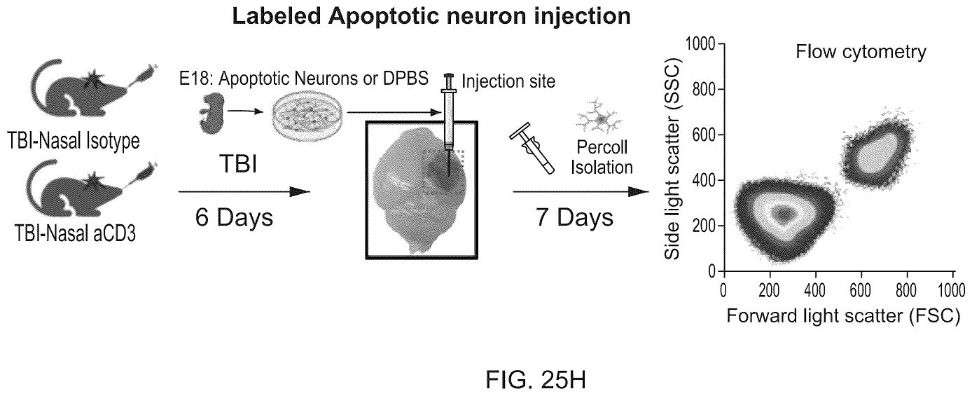

Next, the therapeutic effects of early versus delayed nasal anti-CD3 treatment on behavioral outcomes following TBI were investigated. In the early treatment regimen, nasal anti-CD3 treatment was administered on the same day of CCI which was continued once daily for 7 days, then 3 times weekly for up to 1 month following injury. In the delayed treatment regimen, nasal anti-CD3 was administered at 14 days post-CCI which was continued once daily for 7 days, then 3 times weekly for up to 1-month following injury (FIG. 25A). In the early treatment regimen, there was improvement in motor function and coordination assessed by the rotarod test. Restoration of spatial memory and increased time spent in the target quadrant during the probe trial, assessed by Morris water maze (MWM), were also observed in the TBI-aCD3 group compared to TBI-Iso control (FIG. 25B). In addition, mice treated with nasal anti-CD3 exhibited less anxious behavior, using the open field test. No differences were observed in their exploratory and activity levels compared to TBI-Iso control (FIG. 25B). However, no improvement was observed in the delayed nasal anti-CD3 treatment group compared to TBI-Iso control (FIG. 25C).

To assess the impact of nasal anti-CD3 mAb in a more severe form of TBI, CCI was induced with 3.0 mm tip diameter and 1.5 mm depth of impact over the right parietal cortex as opposed to 1.5 mm tip diameter and 1 mm depth of impact used in the experiments described above. Nasal anti-CD3 mAb was administered on the same day of CCI which was continued once daily for 7 days, then 3 times weekly for up to 1-month following injury (FIG. 25D). There was an improvement in motor function and coordination in the TBI-aCD3 group compared to TBI-Iso control (FIG. 25E), partial restoration in spatial memory and increased time spent in the target quadrant during the probe trial in the nasal anti-CD3-treated mice compared to TBI-Iso control (FIG. 25E). Moreover, mice treated with nasal anti-CD3 exhibited less anxious behavior, though no differences in their exploratory and activity levels compared to TBI-Iso control were observed (FIG. 25E).

Taken together, these data demonstrate that early nasal anti-CD3 mAb improved behavioral outcomes in both moderate and severe subtypes of TBI.

### Nasal Anti-CD3 mAb Modulates Chronic Microglial Inflammatory Response Following TBI.

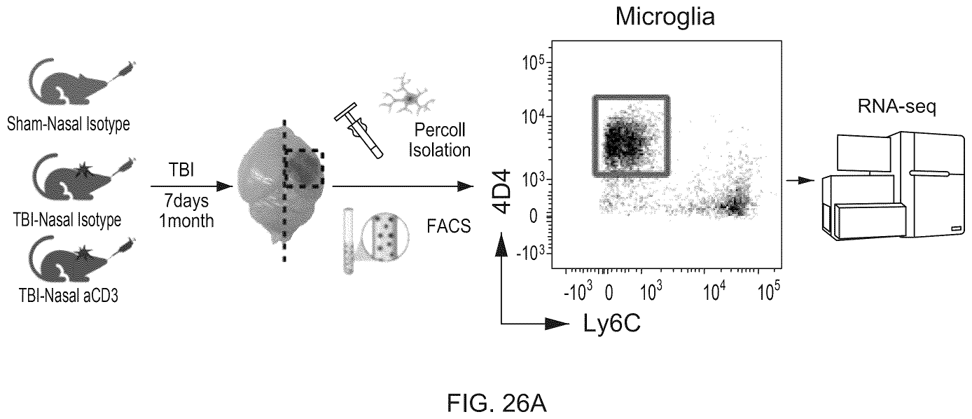

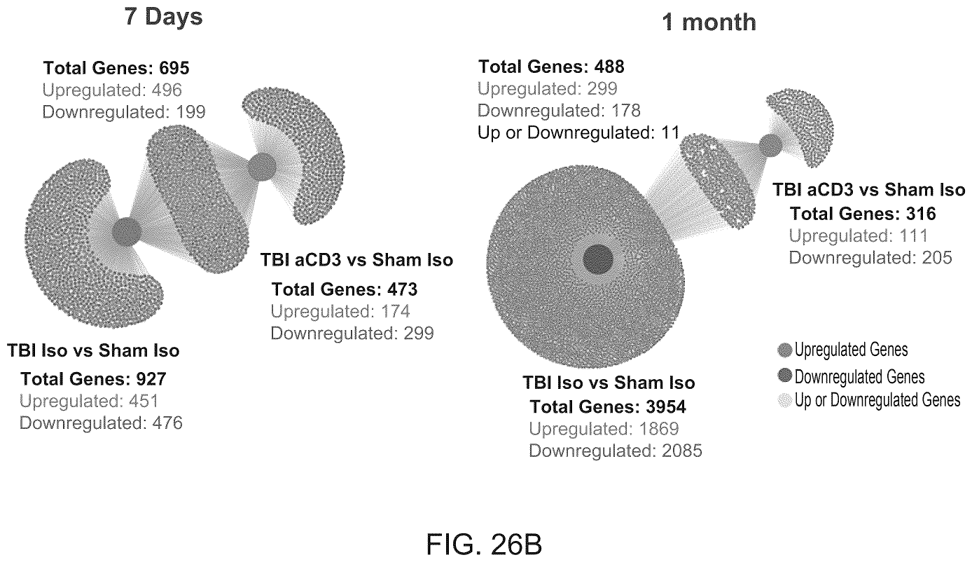

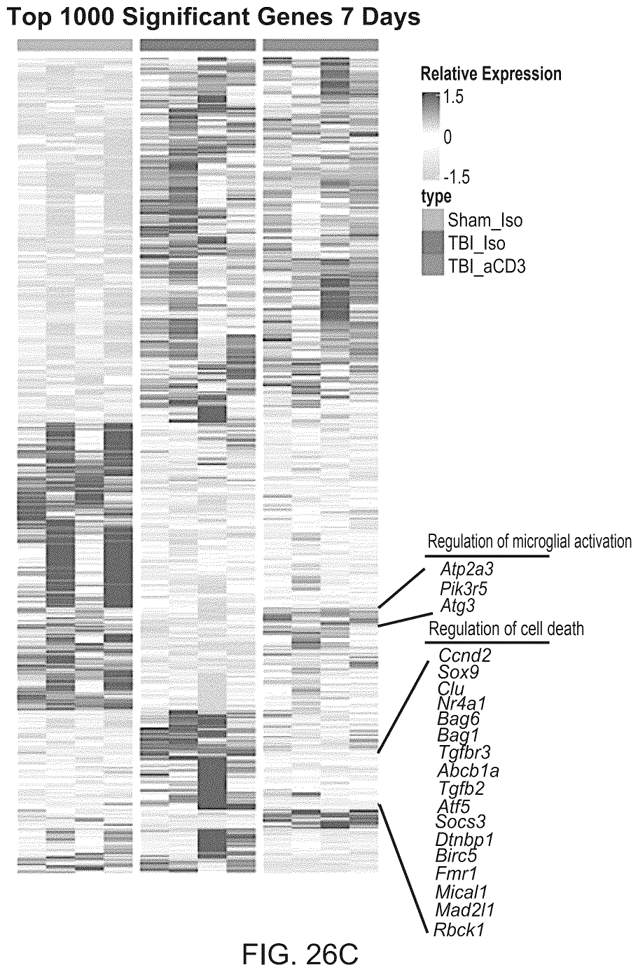

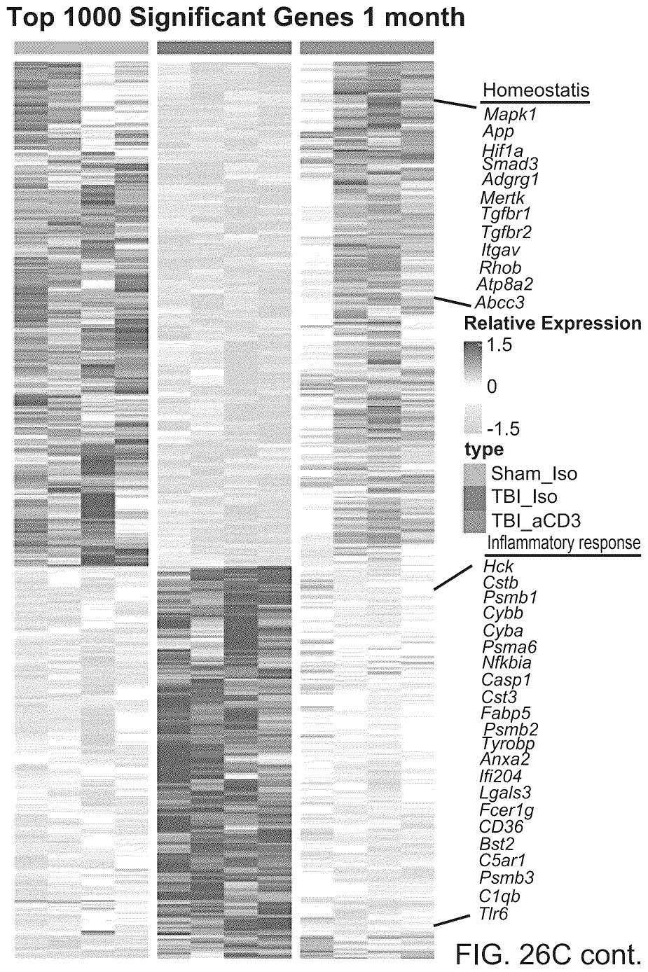

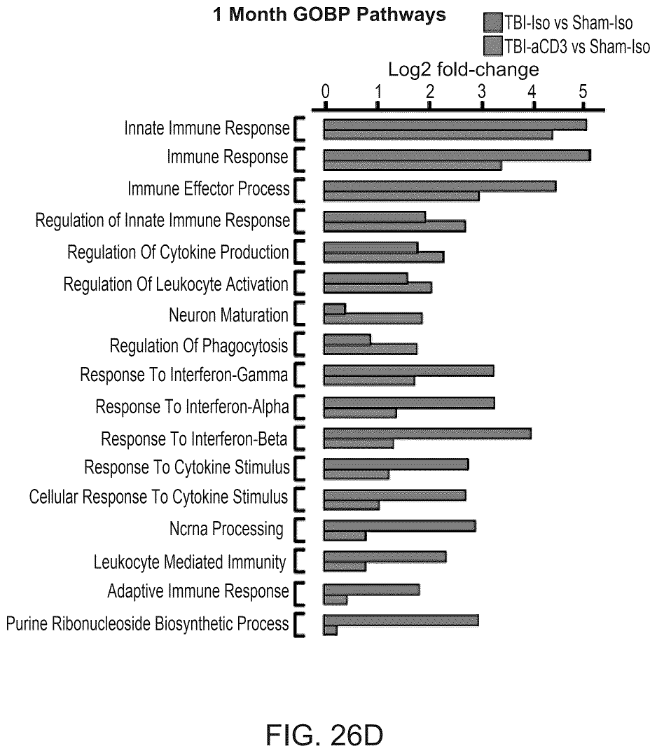

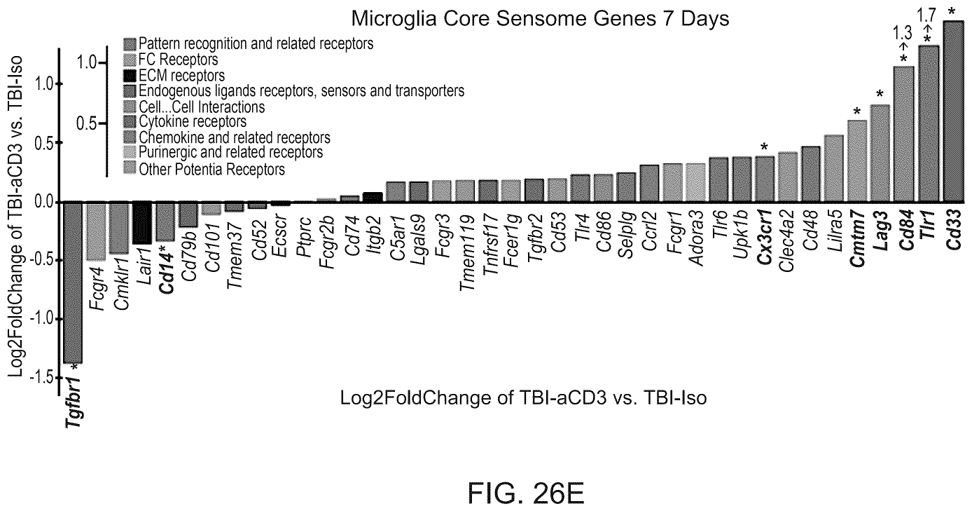

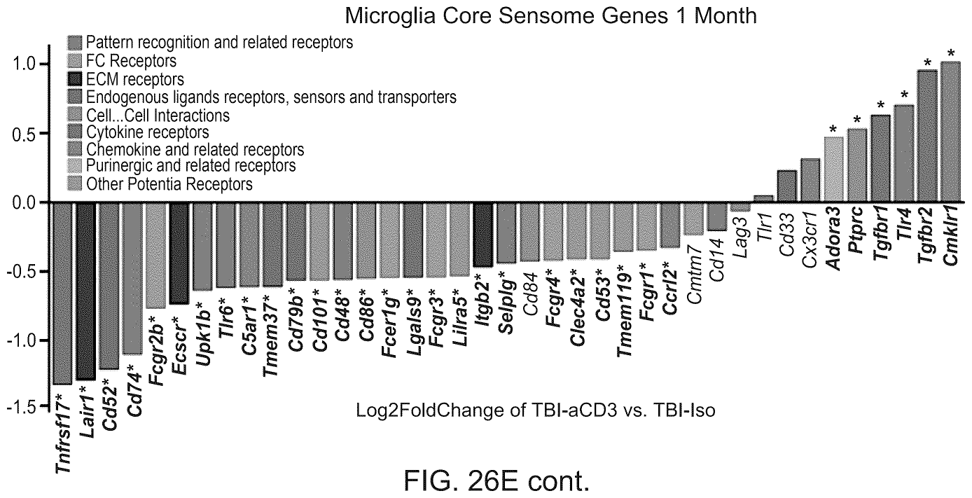

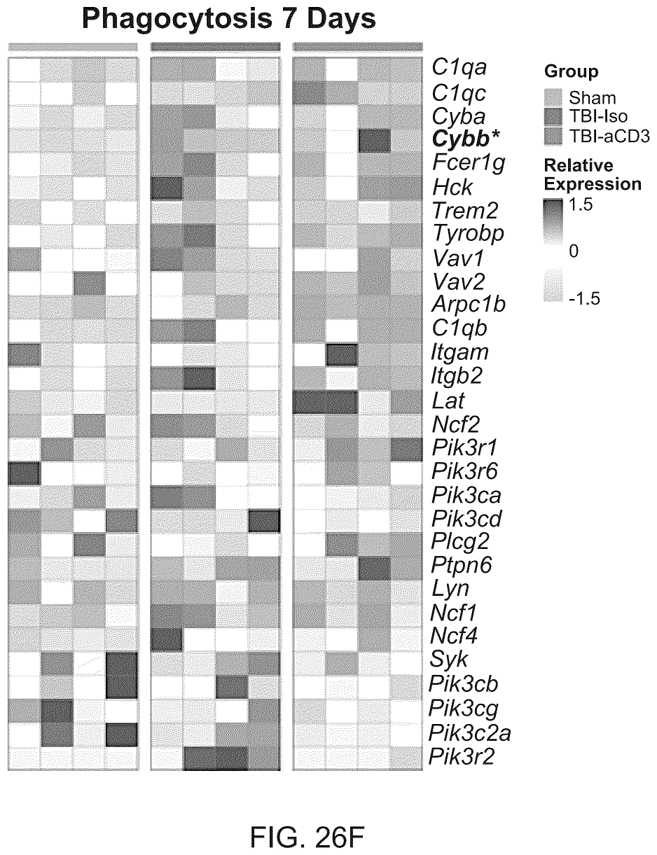

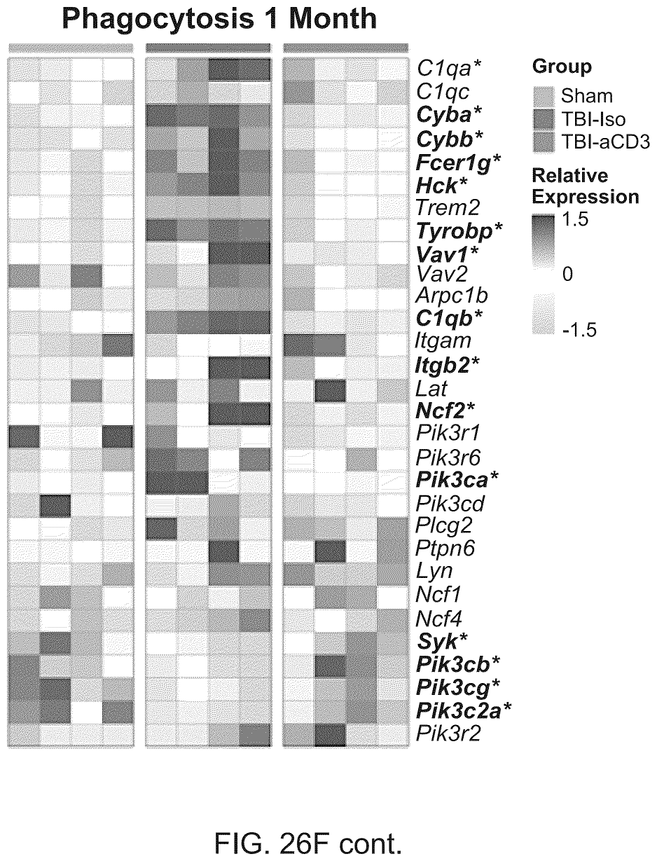

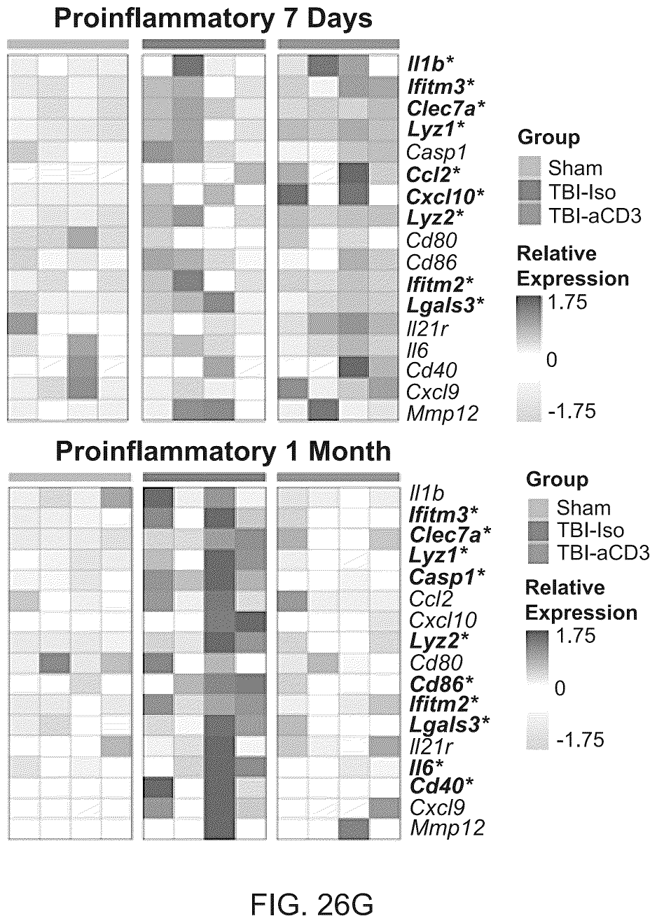

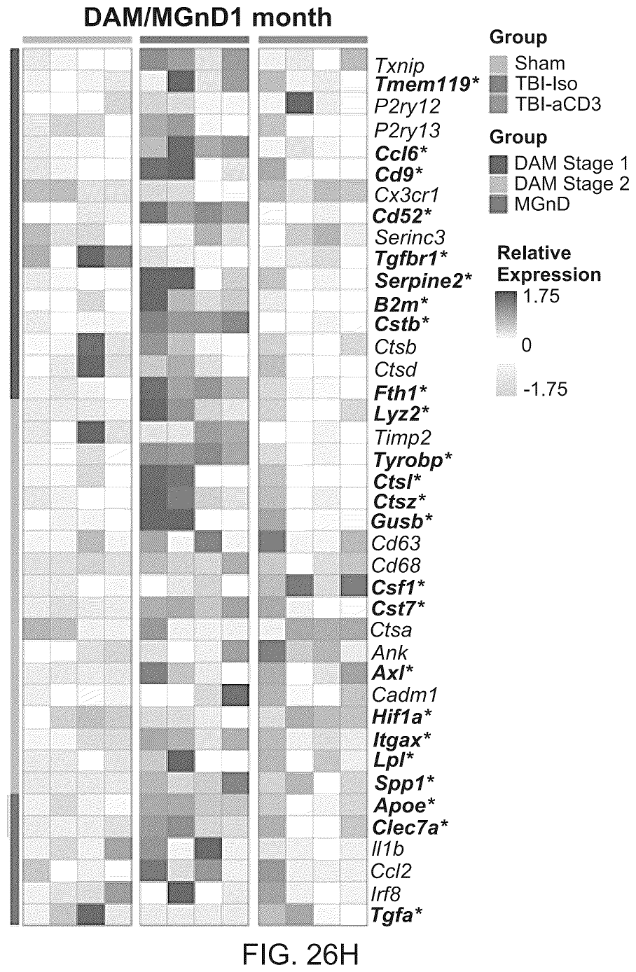

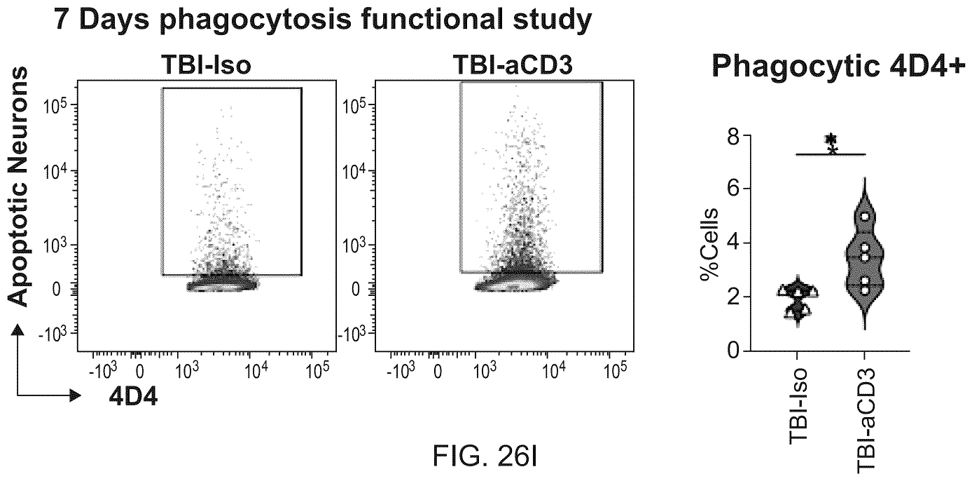

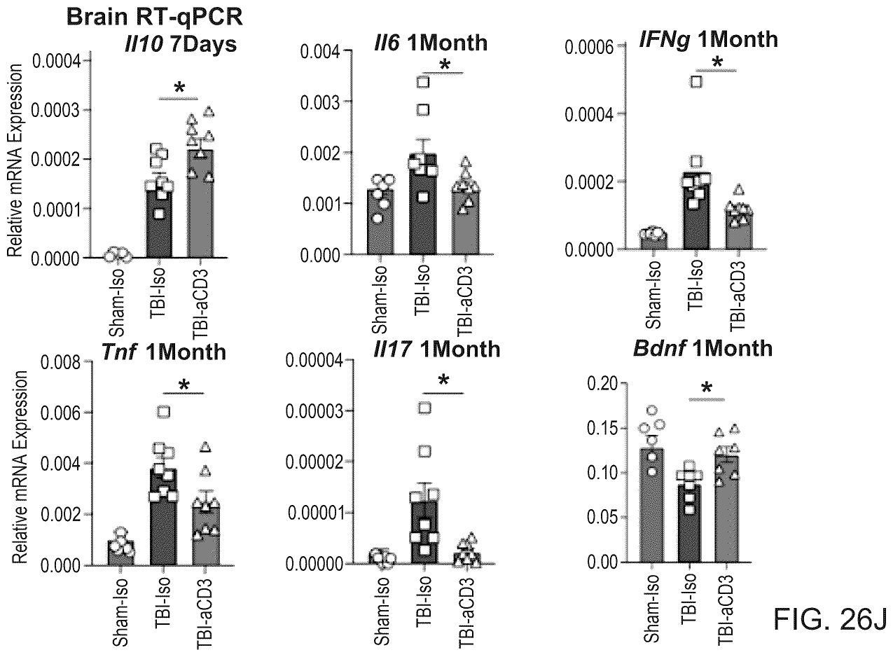

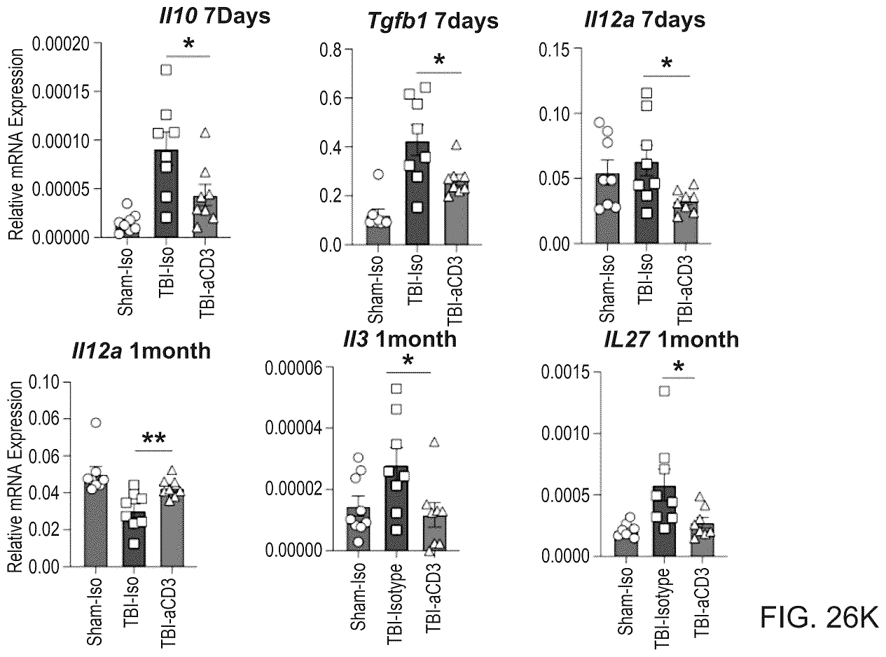

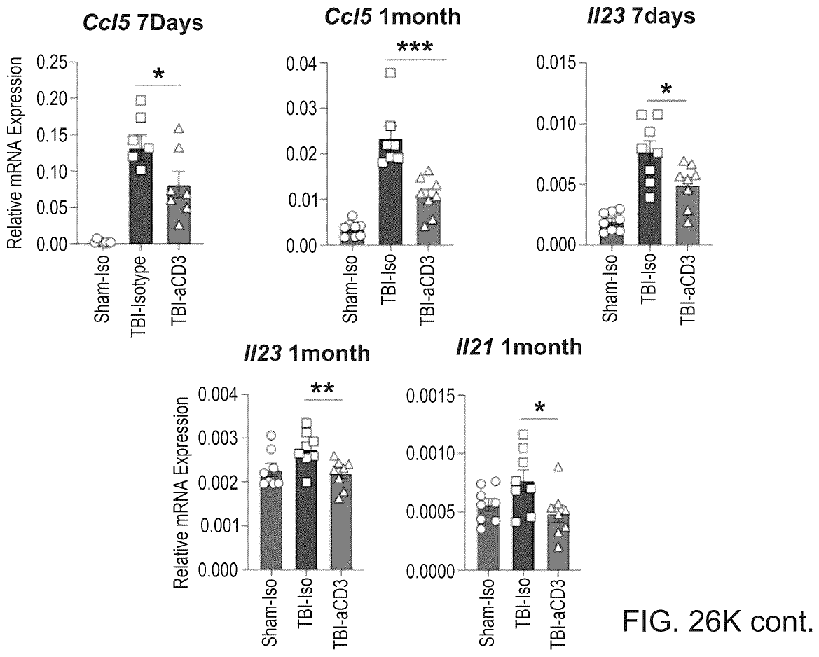

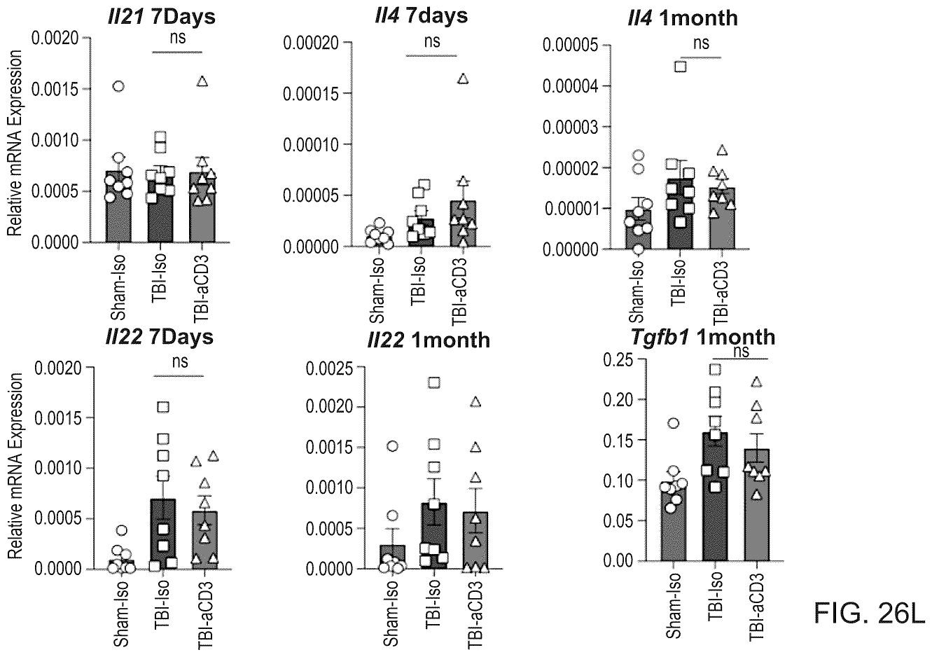

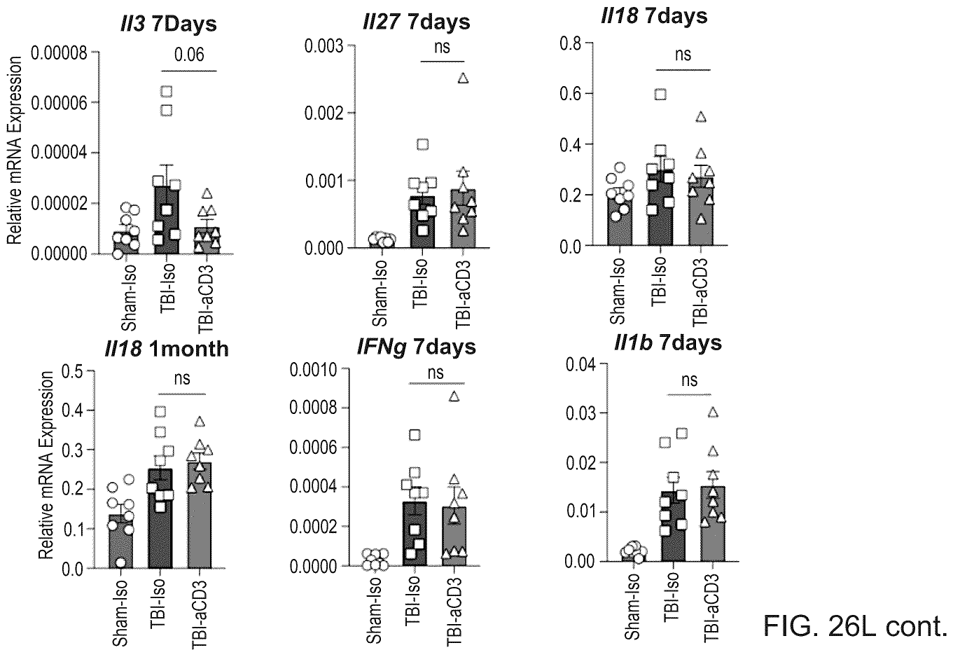

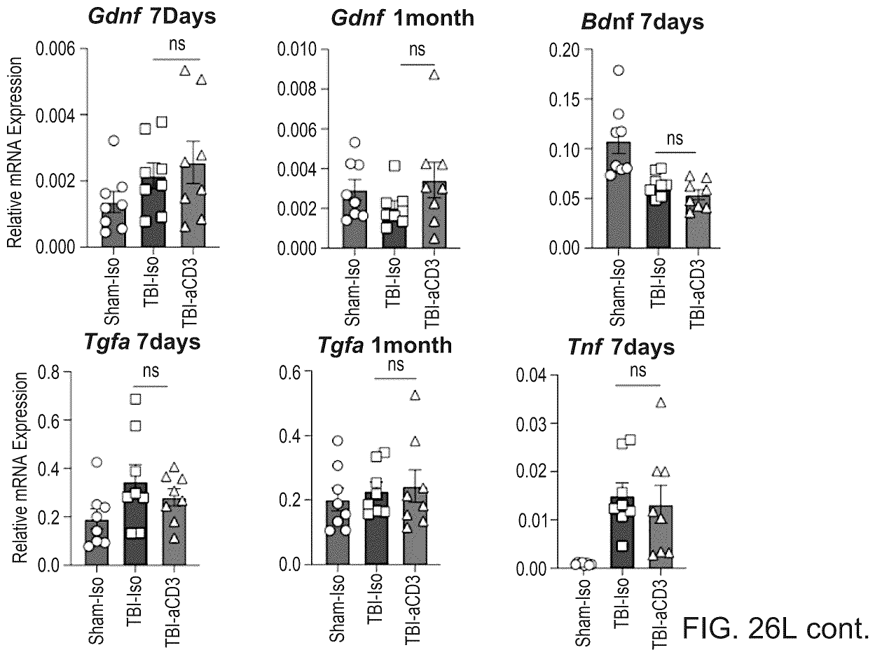

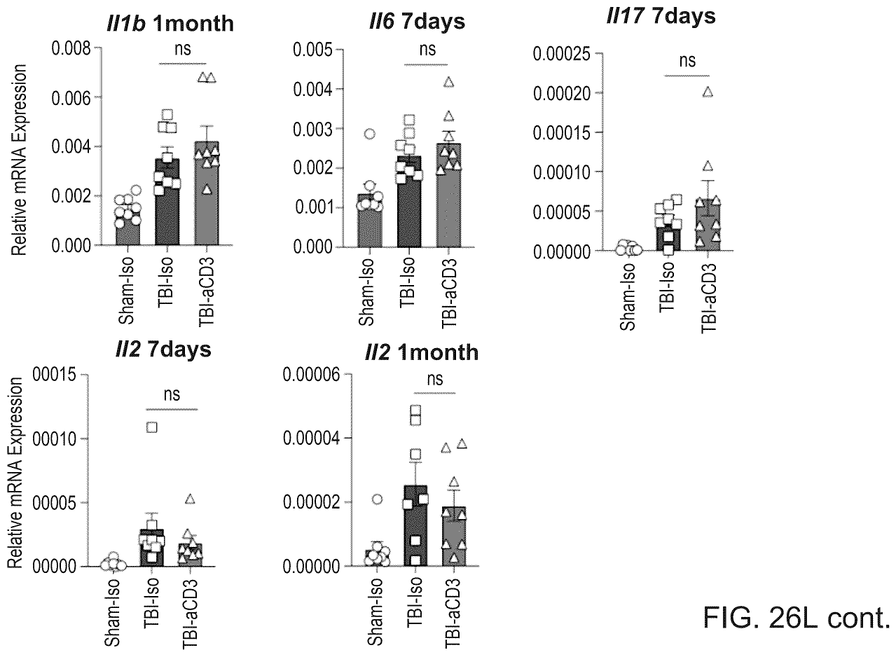

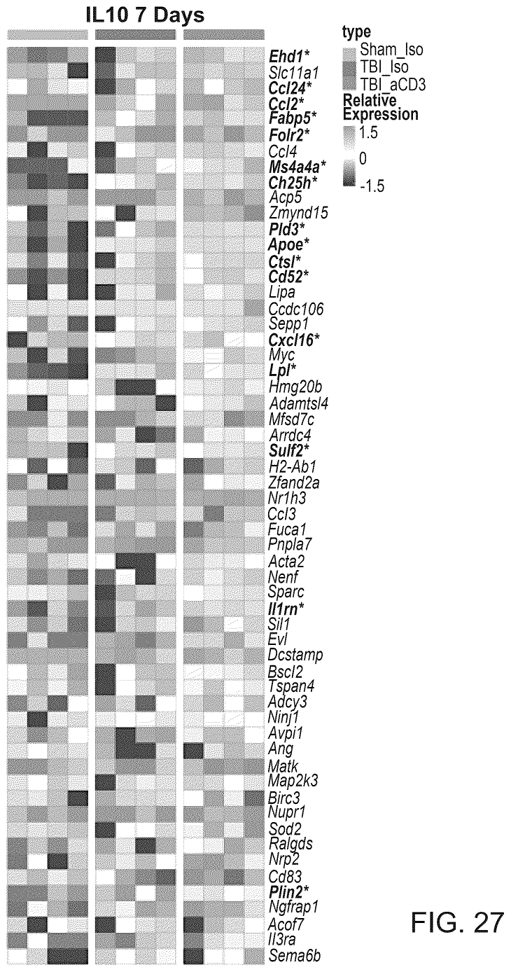

Microglia play a critical role in neuroinflammation, and their activation may contribute to long-term functional deficits after TBI (Jassam et al. Neuron 95, 1246-1265 (2017). To investigate the impact of TBI and nasal anti-CD3 treatment on the microglial inflammatory transcriptomic profile, microglia single-cell suspensions were made from the ipsilateral hemisphere of the mouse brains using the microglia-specific 4D4+ antibody (Krasemann et al. Immunity 47, 566-581 e569 (2017)) at 7 days and 1 month post-CCI(FIG. 26A). When analyzing the highest expressed genes in the Sham-Iso microglia, TBI-Iso microglia and TBI-aCD3 microglia groups, multiple microglial genes including Cx3cr1, HexB, P2ry12 and Tmem119 were among the most expressed genes (FIG. 25F). At 7 days post-injury, 927 differentially expressed genes (DEGs) were found (P<0.05) in microglia isolated from TBI-Iso vs Sham-Iso and only 473 DEGs in TBI-anti-CD3 vs Sham-Iso control. There were 695 DEGs shared between the two comparisons. At 1 month post-injury, the number of DEGs in microglia isolated from TBI-Iso vs Sham-Iso increased to 3954 genes, whereas the number of DEGs in TBI-anti-CD3 vs Sham-Iso control was decreased to 316 genes. In addition, the number of DEGs shared between the comparisons dropped to 488 at 1 month post CCI(FIG. 26B). The heatmap signature of the top 1000 DEGs across three studied groups showed that the TBI-Iso and TBI-aCD3 groups had a similar microglial transcriptomic signature at 7 days, while there was a clear modulation of microglial transcriptomic signature in TBI-aCD3 group towards the Sham-Iso phenotype at 1 month post-injury (FIG. 26C). At 7 days post-CCI, microglia from TBI-aCD3 group had upregulation of genes involved in regulation of microglial activation (Atp2a3, Pik3r5, Atg3) (Jin et al. Biochem Biophys Res Commun 399, 458-464 (2010); Morales-Ropero et al. Glia 69, 842-857 (2021); Friess et al. Mol Brain 14, 87 (2021); Zhao et al. Neurochem Int 157, 105341 (2022)) and downregulation of genes involved in neuroinflammatory responses (Pbx2, Nr4a1) (Wright et al. Genes Immun 9, 419-430 (2008); Rasmussen et al., Mol Brain 10, 43 (2017)), neuronal stress and synaptic dysfunction (Ttr, Dlgap1) (Rasmussen et al. Mol Brain 10, 43 (2017); Wang et al. J Neurosci 34, 7253-7265 (2014)), and regulation of cell death (Ccnd2, Sox9, Clu, Nr4a1, Bag6, Bag1, Tgfbr3, Abcb1a, Tgfb2, Atf5, Socs3, Dtnbp1, Birc5, Fmr, Mical1, Mad2l1, and Rbck1). At 1 month post-CCI, microglia from the TBI-aCD3 group had a homeostatic gene signature similar to the Sham-Iso control, including upregulation of genes involved in homeostasis (Tgfbr1, Tgfbr2, Mapk1, App, Hif1a, Smad3, Adgrg1, Mertk, Itgav, Rhob, Atp8a2, Abcc3). In addition, following nasal anti-CD3 treatment microglia had less inflammatory signature, similar to the Sham-Iso control, including the downregulation of proinflammatory genes such as Tyrobp, Cd36, Cstb, Casp1, Nfkbia, Fcer1g, C5ar1, Psmb3, C1qb and Tlr6 (Kim et al. J Neurosci 28, 4661-4670 (2008); Keren-Shaul et al. Cell 169, 1276-1290 e1217 (2017); Hernandez. et al. Mol Neurodegener 12, 66 (2017). https://doi.org:10.1186/s13024-017-0210-z).

GOBP pathways were performed and the TBI-Iso group and TBI-aCD3 group were compared to the Sham-Iso control group. TBI-Iso was associated with upregulation of inflammatory biological pathways involved in innate and adaptive immune responses including IFN-γ, IFN-α, and IFN-b responses at 7 days and 1 month post-injury, which is consistent with previous reports (Jassam et al., Neuron 95, 1246-1265 (2017)) (FIG. 26D, FIG. 25C). However, TBI-aCD3 treated animals had less upregulation of genes in these proinflammatory pathways and more upregulation in biological pathways involved in regulation of phagocytosis, cytokines production, leukocyte activation, and neuron maturation compared to TBI-Iso at 1 month post-injury (FIG. 26D).

Microglia express genes and a unique transcriptomic signature that allow them to perform microglial sensing, homeostatic, and housekeeping functions, which vary with the physiological and/or pathological state of the brain (Hickman et al. Nat Neurosci 21, 1359-1369 (2018)). To determine the effects of TBI and nasal anti-CD3 on these essential microglial functions, the microglial sensome dataset was examined for genes and pathways involved in each of these functions (Hickman et al. Nat Neurosci 16, 1896-1905 (2013)).

Microglia from nasal anti-CD3 treated group, compared to TBI-Iso, was associated with upregulation of homeostatic and sensing genes that involved in pattern recognition receptors (Tlr1), Fc receptors (Cmtm7), cell-cell interaction (Cd84 and Lag3), and chemoattractant and chemokine receptors (Cx3cr1) at 7 days post-injury (FIG. 26E). Cd33 and Lag3 were among the most significantly DEGs in TBI anti-CD3 treated group compared to TBI-Iso control at 7 days post-injury. Cd33 activity has been implicated in several processes including microglial endogenous ligand receptors and sensors, adhesion processing of immune cells and inhibition of cytokines release by monocytes (Crocker et al., Ann N Y Acad Sci 1253, 102-111 (2012); Crocker et al., Nat Rev Immunol 7, 255-266 (2007)). Lymphocyte activation gene-3 (Lag3) regulates T cell expansion and limits the duration and intensity of the immune response (Workman et al. Eur J Immunol 33, 970-979 (2003)). Moreover, the TBI anti-CD3 treated group showed downregulation of key regulators of microglial proinflammatory responses to injury including CD14 (Janova et al. Glia 64, 635-649 (2016)) at 7 days post-injury. At 1 month post-injury, nasal anti-CD3 treatment was associated with upregulation of several TGFβ-signaling genes including Smad3, Tgfbr1 and Tgfbr2 compared to TBI-Iso control (Zoller et al., Nat Commun 9, 4011 (2018)). TGF-β is required for maintaining the microglial homeostatic state (Butovsky et al. Nat Neurosci 17, 131-143 (2014)) and modulating microglial mediated inflammation after acute brain injury (Taylor et al. J Clin Invest 127, 280-292 (2017)). In addition, nasal anti-CD3 treatment downregulated sensing genes involved in ECM (Lair1, Ecscr, Itgb2), cytokine receptor (Tnfrsf17), FC receptor (Fcgr1, Fcgr4, Fcgr3, and Fcer1g) and pattern recognition receptors (Cd74, Tlr6, Selplg) compared to TBI-Iso control (FIG. 26E).

TBI results in large amounts of myelin and cell debris and microglia and macrophages play an important role in debris removal (Jassam et al., Neuron 95, 1246-1265 (2017)). Expression levels of microglial genes involved in phagocytosis were analyzed and TBI was found to be associated with upregulation of the microglial phagocytotic gene signature including Cybb, C1qa, C1qb, Cyba, Fcer1g, Itgb2, and Tyrobp, particularly at 1 month post injury. Conversely, nasal anti-CD3 treatment was associated with upregulation of the microglial chemotactic and phagocytic transcriptomic profile at 7 days post-CCI and downregulation at 1-month post-injury (FIG. 26F). Cybb, which encodes the gp91-phox component of the phagocyte oxidase enzyme complex and is involved in generating reactive oxygen and superoxide species (Frazao et al. J Cell Biochem 116, 2008-2017 (2015)) was upregulated at 7 days, but downregulated 1 month after CCI, in mice treated with nasal anti-CD3 mAb. Other critical regulators of microglial phagocytosis such as Syk and Pik3cg remained upregulated in the nasal anti-CD3 group at 1 month post-CCI. Nasal anti-CD3 treatment also upregulated Mertk, a functional regulator of myelin phagocytosis (Healy et al. J Immunol 196, 3375-3384 (2016)) at 1 month post-CCI compared to TBI-Iso control.

Several studies have reported a link between the chronic microglial pro-inflammatory response following TBI and chronic neurodegeneration (Jassam et al., Neuron 95, 1246-1265 (2017)). Thus, the expression levels of several pro-inflammatory microglial genes (FIG. 26G) and disease associated (DAM) (Keren-Shaul et al. Cell 169, 1276-1290 e1217 (2017) or neurodegenerative (MgnD) (Krasemann et al. Immunity 47, 566-581 e569 (2017)) microglia genes post-injury (FIG. 26H) were analyzed. TBI was associated with upregulation of several proinflammatory genes (Ifitm3, Clec7a, Ccl2, Lgals3, IL6, Casp1, CD86, Lyz1, Lyz2, CD40) and DAM 1 and 2 genes (Tmem119, B2m, Cstb, Cst7, Fth1, Ccl6, Cd9, Cd52, Tyrobp) at 1 month post-CCI compared to Sham-Iso control. Importantly, these genes were downregulated by nasal anti-CD3 treatment (FIGS. 26G-26H). In addition to modulating chronic inflammation, nasal anti-CD3 upregulated genes involved in synaptic pruning and remodeling such as Cx3cr1 at 7 days post-injury compared to TBI-Iso control (Cornell et al. Neural Regen Res 17, 705-716 (2022) (FIG. 26E).

To assess the phagocytosis capacity of microglia after TBI (with and without treatment), an in-vivo experiment was performed where the TBI induced lesion was injected with either labelled apoptotic neurons or DPBS on day 6 post-injury. In line with the microglial transcriptomic data, anti-CD3 treated animals had higher microglial phagocytic capacity to uptake the apoptotic neurons at 16 hours post injection compared to TBI-Iso group (FIG. 26I; FIGS. 25G-I).

Consistent with the microglial transcriptomic data, RT-qPCR from the ipsilateral hemisphere showed that TBI was associated with an increase in pro-inflammatory cytokines (Il12a, Il23 and Ccl5) at 7 days and (Il23, Ccl5, IFN-γ, Il6, Il17, Il27 and TNF) 1 month post-injury. Nasal anti-CD3 treatment increased the anti-inflammatory cytokine Il10 at 7 days and reduced the expression of several key proinflammatory cytokines (Il6, IFN-γ, TNF, Il17, Ccl5, Il23 and Il12a) compared to TBI-Iso control at 1 month post-injury (FIG. 26J; FIG. 26K). Of note, mice treated with nasal anti-CD3 showed upregulation of brain-derived neurotrophic factor (Bdnf), neurotrophins that have a critical role in neuronal survival and that are involved in synaptic plasticity, learning and memory, compared to the TBI-Iso control, at 1 month post-injury (FIG. 26J).

Taken together, these data indicate that nasal anti-CD3 modulated the microglial proinflammatory response post-TBI by upregulating microglial homeostatic, sensing and phagocytic genes and increasing microglial phagocytic capacity at the acute stage of injury and by downregulating pro-inflammatory and DAM/MgnD microglial genes at the chronic stage of injury.

### Nasal Anti-CD3 mAb Ameliorates TBI in an IL-10-Dependent Manner.

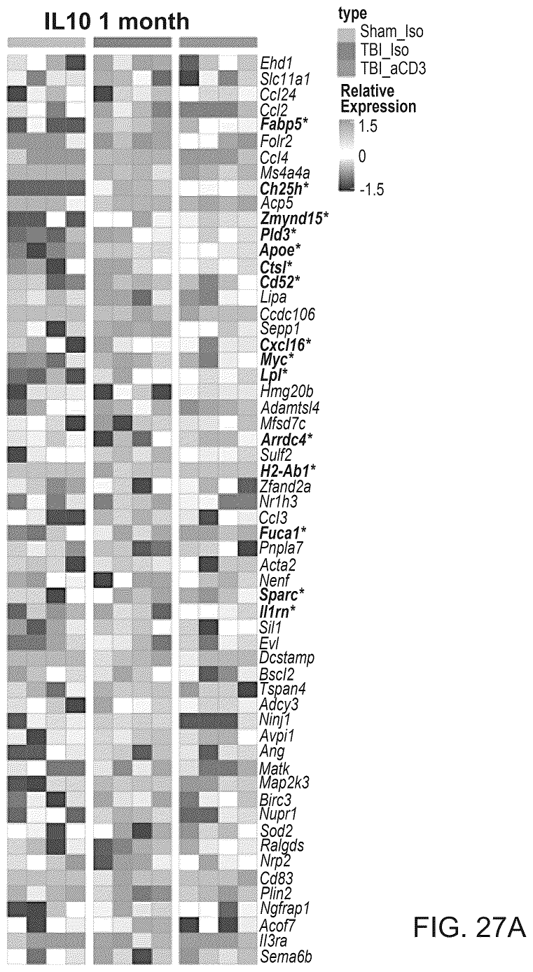

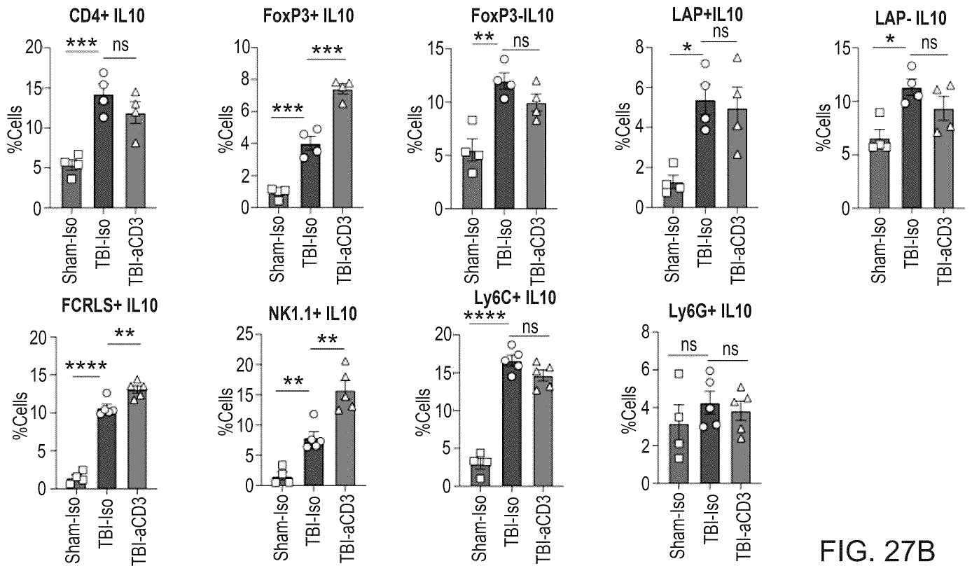

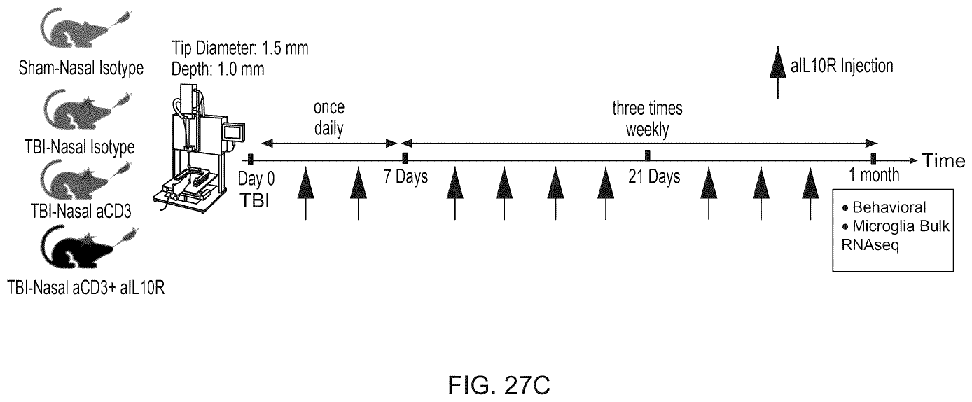

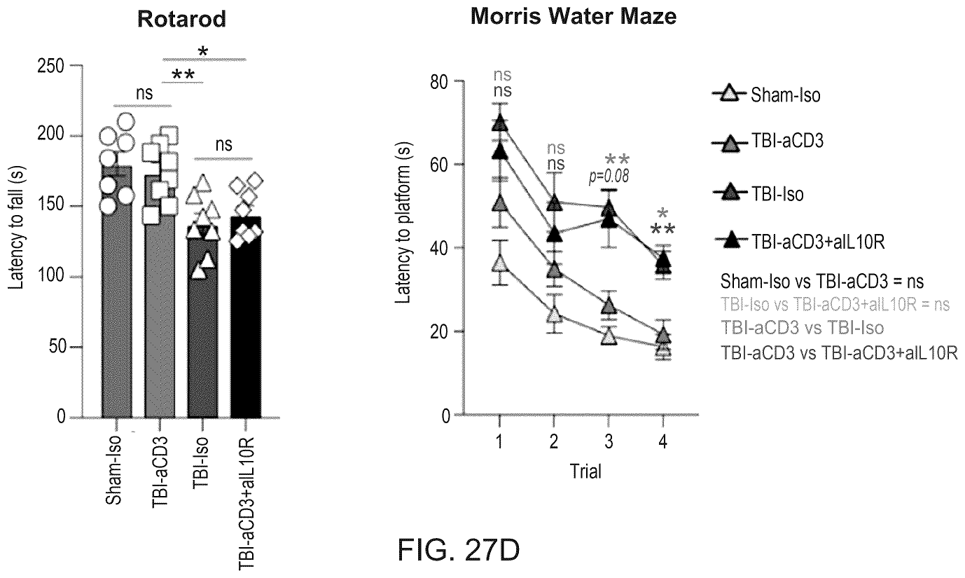

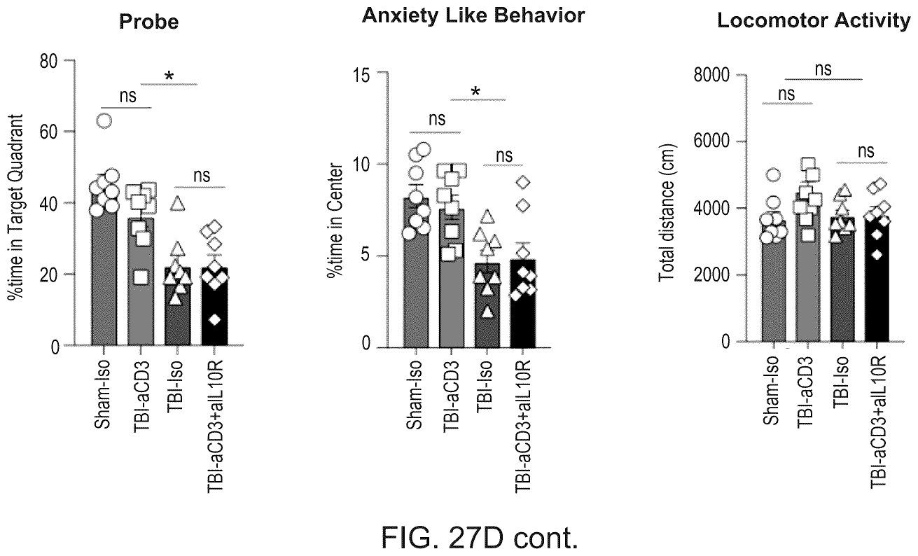

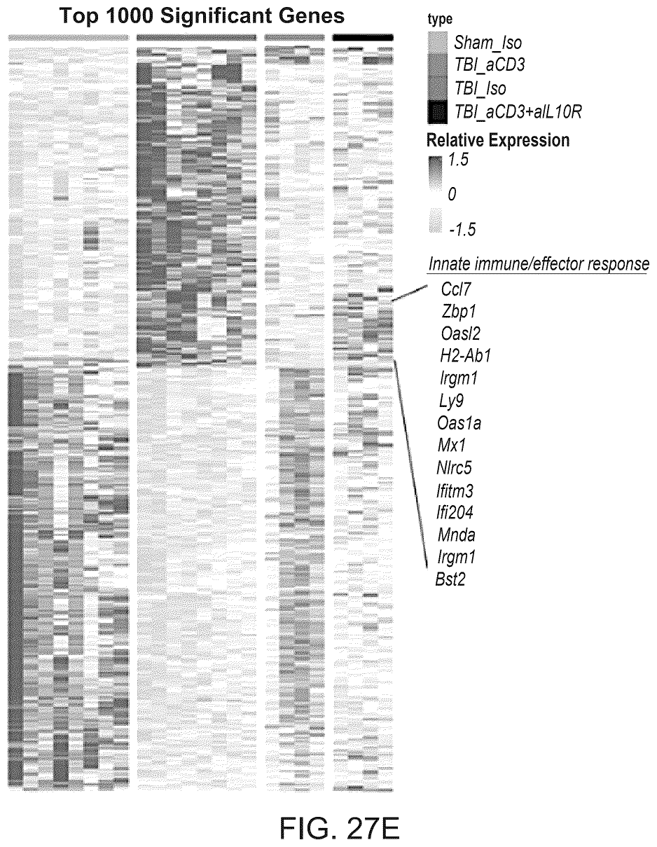

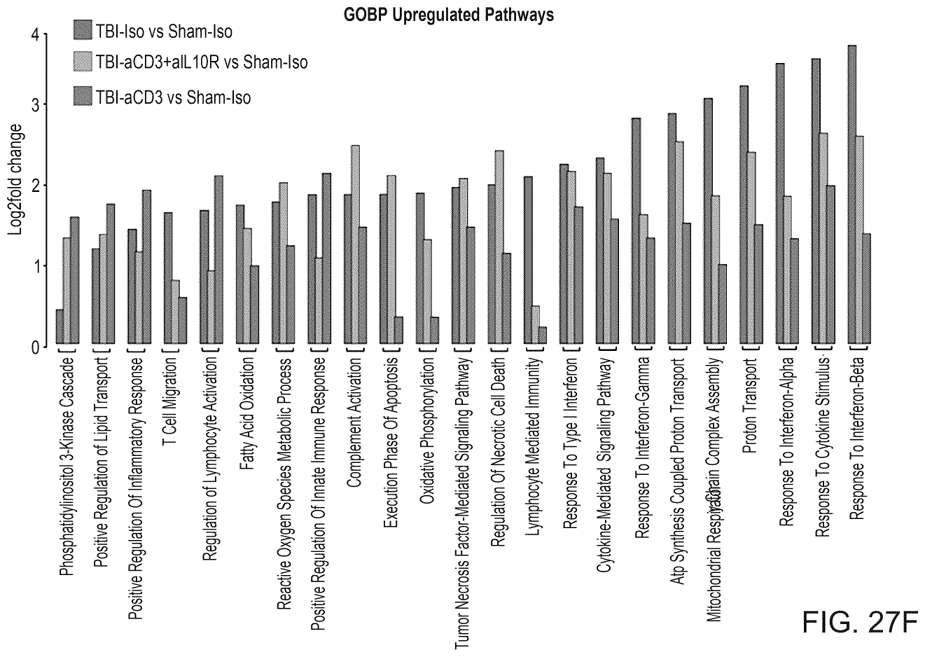

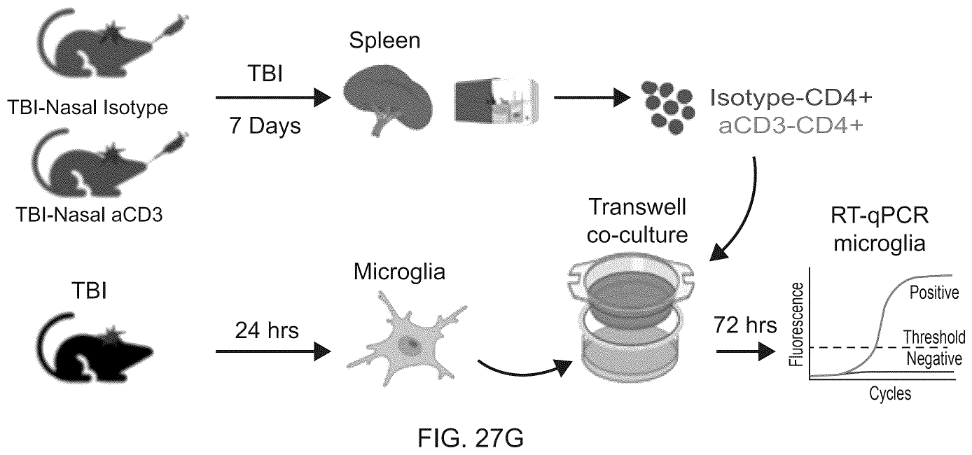

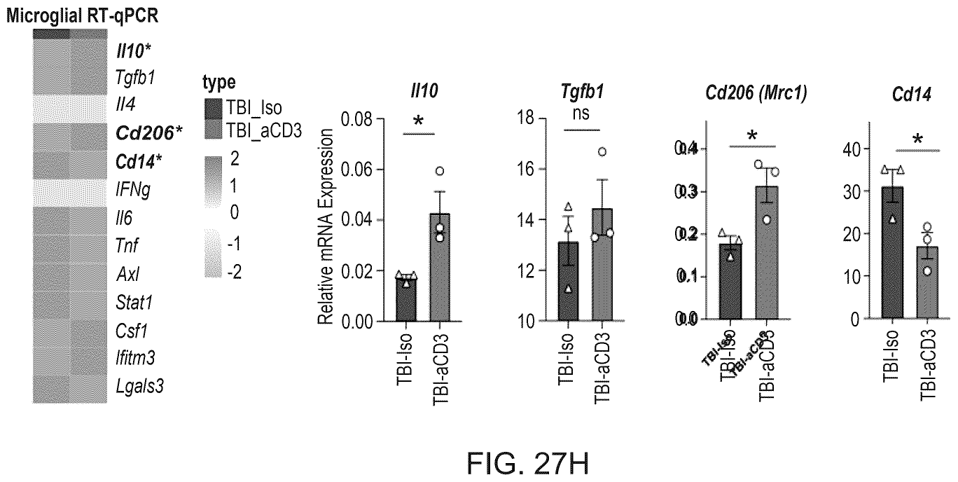

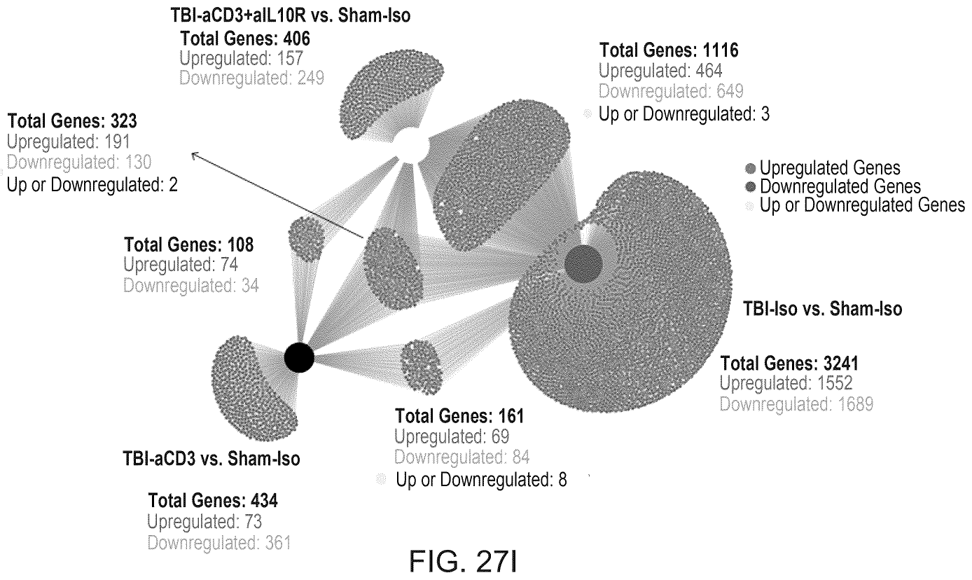

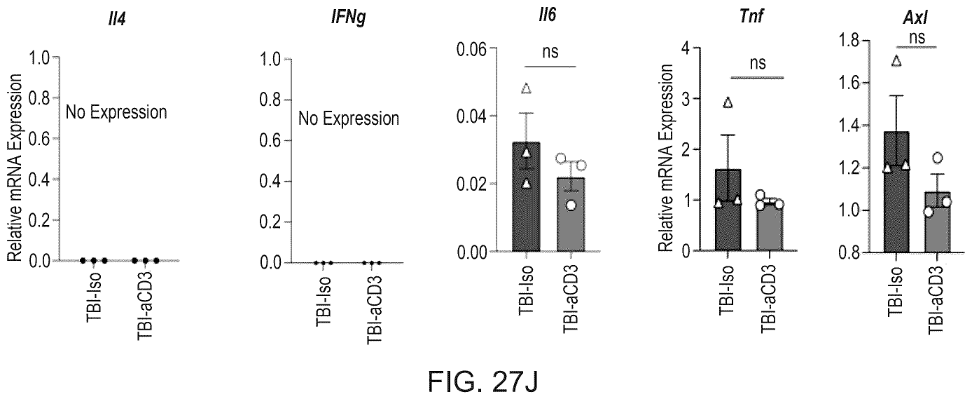

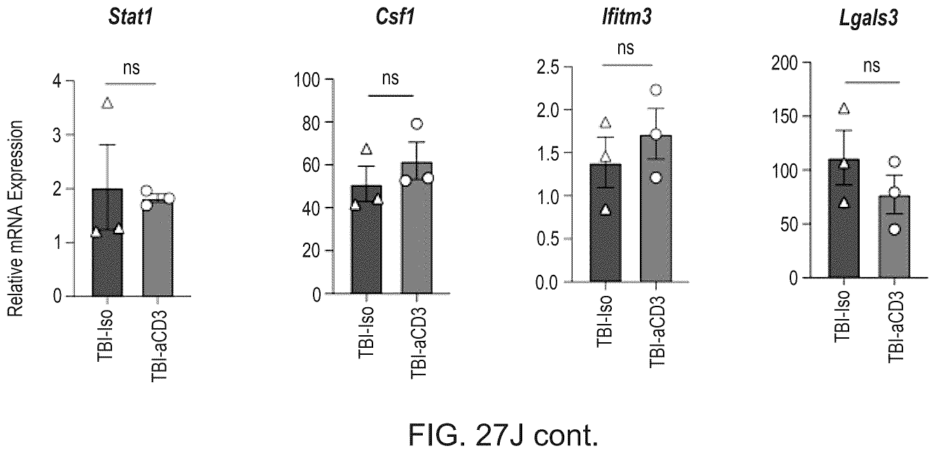

It has been previously shown that nasal anti-CD3 treats an autoimmune model of progressive multiple sclerosis by inducing IL-10+ Tregs (Mayo et al. Brain 139, 1939-1957 (2016)). In this work, IL-10 expression was increased in the ipsilateral hemisphere of TBI-aCD3 treated animals compared to Sham-Iso and TBI-Iso controls at 7 days post-CCI(FIG. 26J). There was also upregulation of IL-10-cytokine gene expression in microglia of TBI-aCD3 group compared to Sham-Iso and TBI-Iso controls at 7 days post-CCI(FIG. 27A). Thus it was investigated whether IL-10 plays a role in the beneficial effects of nasal anti-CD3 following an acute brain injury. The percentage of cells expressing IL-10 in CNS immune cells, including total CD4+, Treg subtypes (CD4+FoxP3+, CD4+LAP+, CD4+FoxP3−, and CD4+LAP− cells), NK cells, microglia, infiltrating monocytes, and neutrophils from the ipsilateral hemisphere of the brain were examined by flow cytometry at 7 days post-TBI in Sham-Iso, TBI-Iso, and TBI-aCD3 groups. TBI Iso and TBI-aCD3 groups had an increase in IL-10 expression in total CD4+, CD4+FoxP3+, CD4+FoxP3−, CD4+LAP+, CD4+LAP−, microglia, infiltrating monocytes, and NK cells at 7 days post-CCI compared to Sham-Iso control. However, nasal anti-CD3 treatment increased in the expression of IL-10 in FoxP3+ Treg population as well as microglia and NK cells at 7 days post-CCI compared to TBI-Iso control (FIG. 27B).

Then the effect of blocking the IL-10 receptor on the behavioral outcomes post-CCI (with and without nasal anti-CD3 treatment) was examined by administrating anti-IL10-receptor (aIL-10R) blocking antibody intraperitoneally every three days post-injury (FIG. 27C). The effect of blocking the IL-10 receptor was measured in Sham-Iso, TBI-Iso, TBI-aCD3 and TBI-aCD3+aIL10R groups. The improvement in motor and coordination functions, spatial memory, and the anxiety-like behavior observed in TBI-aCD3 was abrogated by blocking IL-10 (TBI-aCD3+aIL10R group) (FIG. 27D).

To investigate the impact of blocking the IL-10 receptor on the microglial inflammatory transcriptomic profile, microglia single-cell suspensions were isolated from the ipsilateral hemisphere using the microglia-specific 4D4+ antibody (Krasemann et al. Immunity 47, 566-581 e569 (2017)) at 1 month post-CCI. TBI-aCD3+aIL10R vs. Sham-Iso shared Il16 DEGs (P<0.05) with TBI-Iso vs Sham-Iso, while TBI-aCD3 vs. Sham-Iso had only 161 DEGs that overlapped with TBI-Iso vs Sham-Iso (FIG. 27I). The modulatory effect of nasal anti-CD3 on microglia (FIG. 26C-26D)) was abrogated by blocking IL-10 as shown in the heatmap signature of the top 1000 DEGs across the groups (FIG. 27E; Supplementary Ta. At 1-month post-CCI, similar to TBI-Iso control, microglia from TBI-aCD3+aIL10R group had a more proinflammatory signature compared to Sham-Iso and TBI-aCD3 group (FIG. 27E). Then, GOBP pathways was performed and it was found that the TBI-aCD3+aIL10R group was associated with upregulation of genes involved in pro-inflammatory pathways, including the response to IFN-γ (Ccl7, Ifitm3, H2-Abl, Irgm1, Bst2), IFN-α (Ifitm3, Bst2, Ifi204), and IFN-b (Mnda, Ifitm3, Irgm1, Bst2, Ifi204) and complement activation. Similar to TBI-Iso, the TBI-aCD3+aIL-TOR group showed more upregulation in ROS, necrotic cell death and apoptotic pathways compared to TBI-aCD3 group (FIG. 27F).

Taken together, these data demonstrate that nasal anti-CD3 induced CD+4 Tregs modulated the microglial response and improved outcomes post-injury in an IL-10 dependent manner.

### Tregs Suppress Microglia Activation In Vitro.

To further investigate the interaction between Tregs and microglia following TBI, an ex-vivo transwell co-culture system was employed, in which microglia were isolated from the ipsilateral hemisphere of CCI mice 24 hours after injury and Tregs were isolated from spleens of a separate cohort of mice subjected to CCI and treated with nasal anti-CD3 or isotype control for 7 days (FIG. 27G). Microglia were placed in the lower chamber and Tregs in the upper chamber and RT-qPCR of microglia was performed 72 h after incubation. There was an increase in IL-10 expression in TBI-aCD3 microglia but no differences in the expression levels of other Treg-related cytokines such as IL4 and Tgfb1 (FIG. 27H; FIG. 27J). There was also an increase in the expression of the microglial anti-inflammatory marker Cd206 and decrease of the pro-inflammatory marker Cd14. No differences in other pro-inflammatory cytokines including IL-6 and TNF, and pro-inflammatory microglial markers including Axl, Stat1, Csf1, and Ifitm3 compared to TBI-iso were found (FIG. 27H; FIG. 27J). These findings demonstrate that Tregs induced by the nasal administration of anti-CD3 suppress microglia activation in vitro in an IL-10 dependent fashion.

### CD4+FoxP3+ Regulatory T Cells Attenuate the Innate Inflammatory Response and Improve Behavioral Outcomes Following TBI.

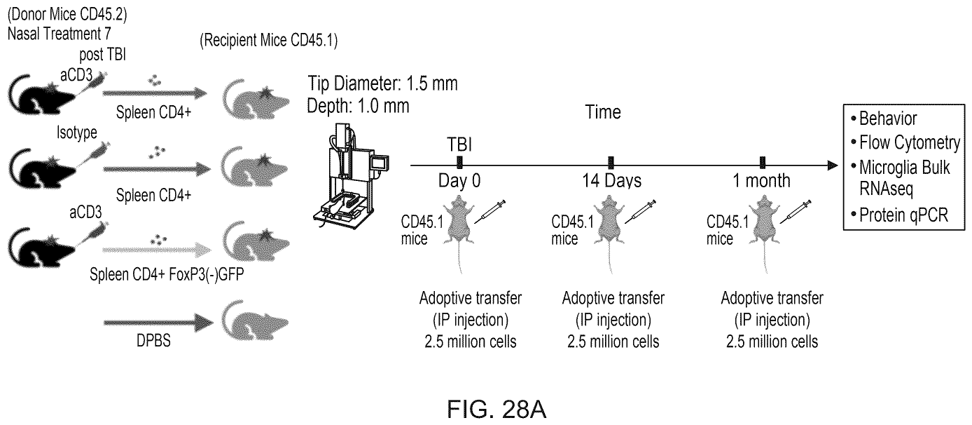

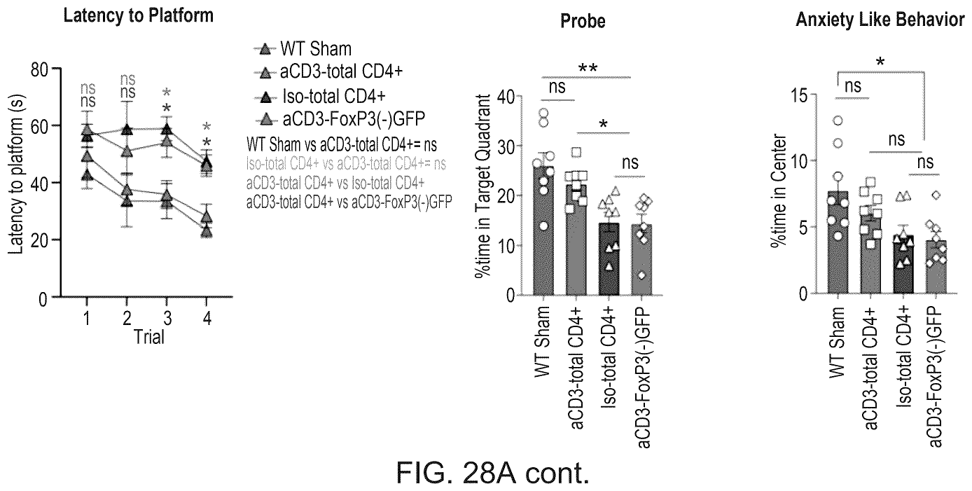

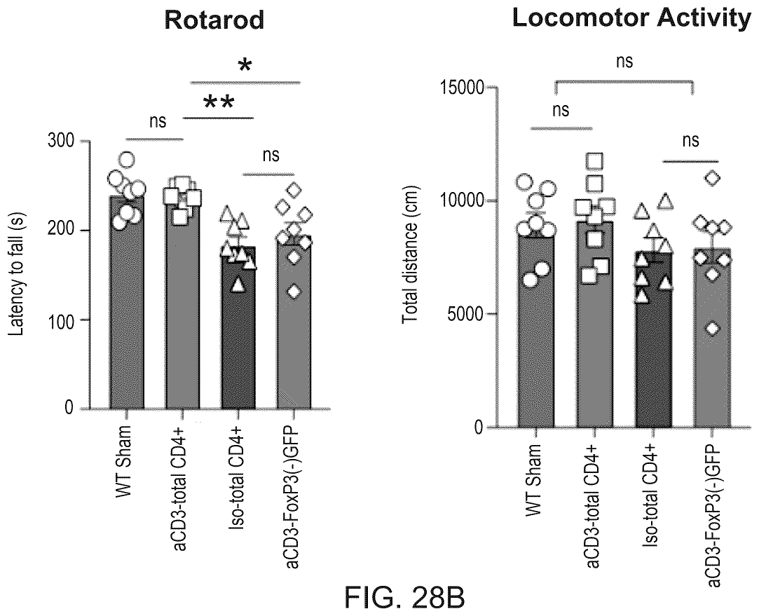

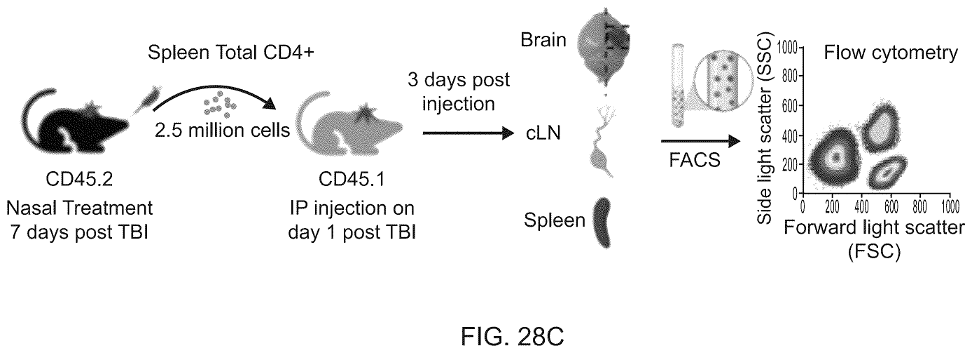

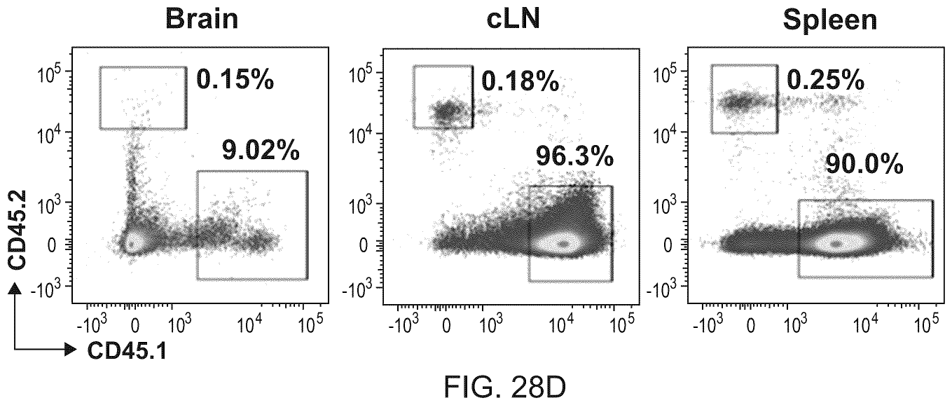

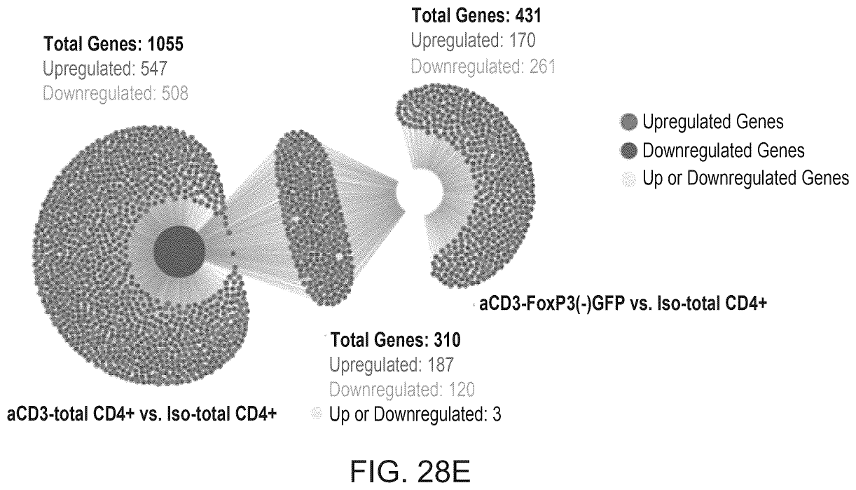

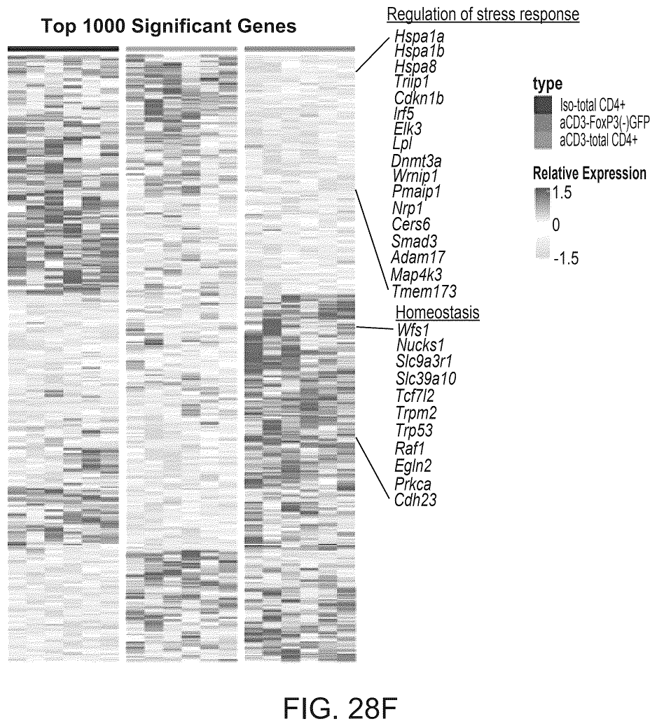

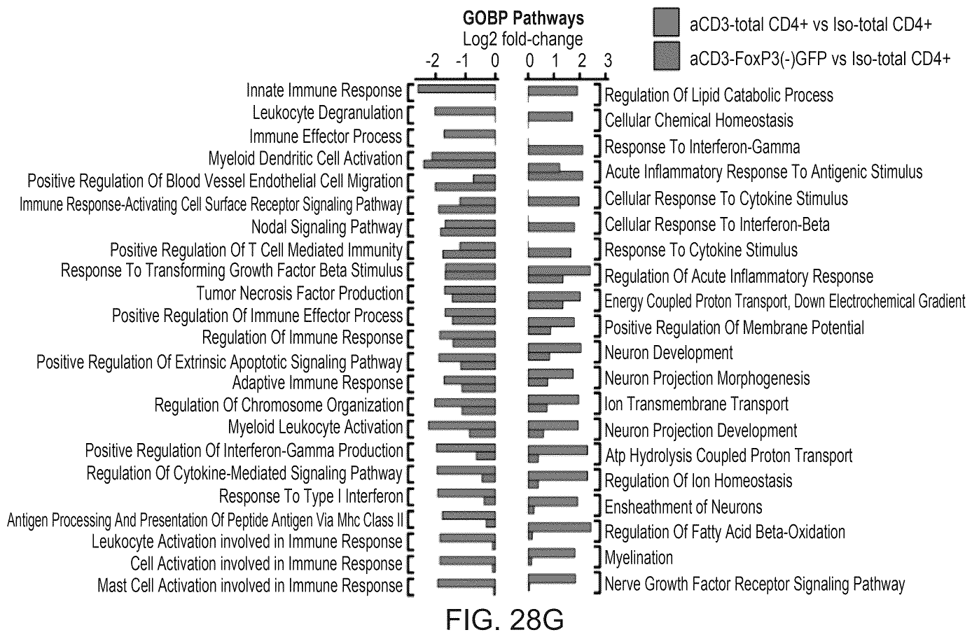

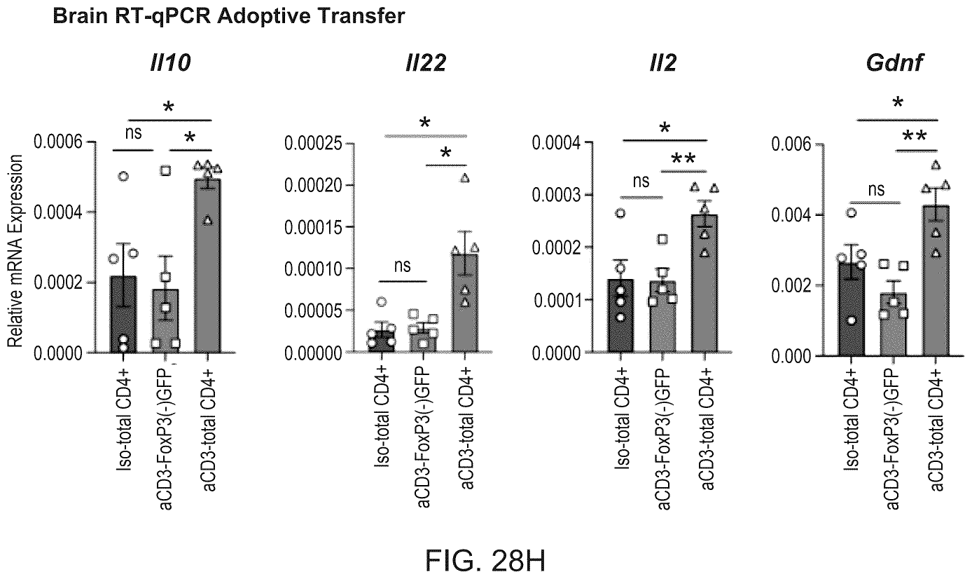

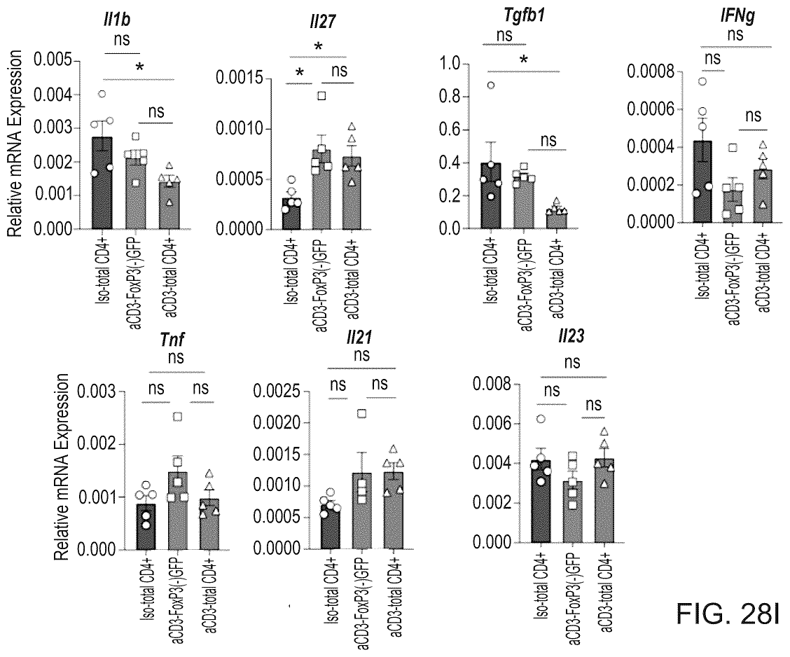

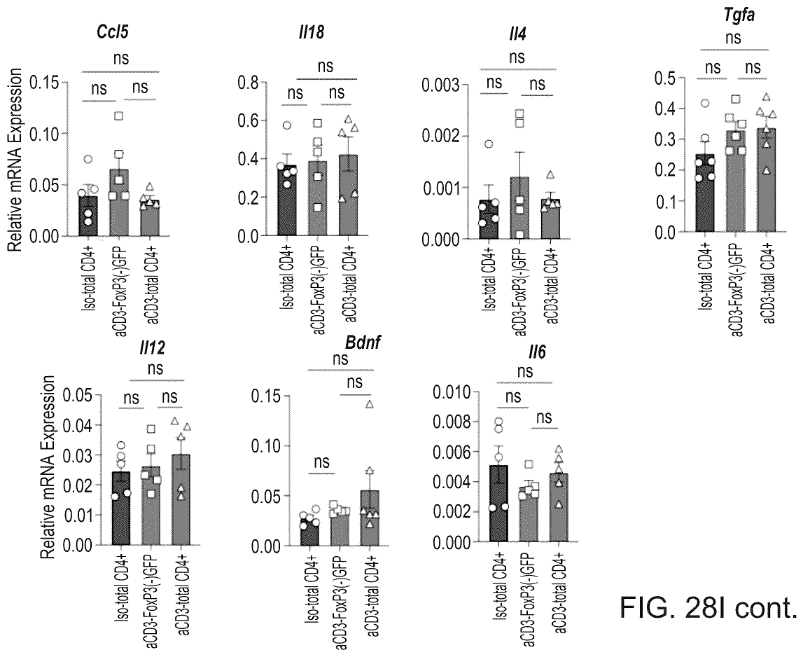

CD4+FoxP3+ cells were increased in TBI-aCD3 treated animals (FIG. 24I and FIG. 27B). Thus, their effects on behavior and microglial transcriptomic profiles following injury was assessed in adoptive transfer experiments. Total splenic T cells (CD45.2+CD4+) isolated from TBI-Iso (Iso-total CD4+) and TBI-nasal anti-CD3 treated mice (aCD3-total CD4+), and CD45.2+CD4+FoxP3GFP negative cells isolated from anti-CD3 treated animals (aCD3-FoxP3(−) GFP) post-CCI were intraperitoneally transferred into untreated but CCI-injured congenic CD45.1-expressing mice. Adoptive transfer was performed at 3 timepoints post-CCI with each mouse receiving 2.5 million cells per injection (FIG. 28A). Behavior was then measured using rotarod, MWM and open field tests. There was improvement in motor function and coordination, restoration of spatial memory and increased time spent in the target quadrant during the probe trial in mice that received total CD4+ T cells from anti-CD3-treated mice as compared to mice that received CD4+ T cells depleted of FoxP3 Tregs at 1 month post-CCI(FIG. 28B). No improvement in anxiety-like behavior or locomotor activity was found between groups.

Then adoptively transferred cells were tracked in recipient mice by transferring cells from CD45.2 mice into CD45.1 mice. Flow cytometric analyses of CD45.2-expressing cells in the brain, cLN, and spleen of recipients was preformed (FIG. 28C). CD45.2 transferred cells were found in all organs analyzed (FIG. 28D).

To investigate the impact of total CD4+ and CD4+FoxP3(−)GFP negative cells on the microglial transcriptomic profile post-injury, microglia were isolated from the ipsilateral hemisphere using the microglia-specific 4D4+ antibody (Krasemann et al. Immunity 47, 566-581 e569 (2017)).

from Iso-total CD4+, aCD3-total CD4+, and aCD3-FoxP3(−)GFP groups and performed bulk RNA-seq at 1 month post-CCI. 1055 DEGs (P<0.05) were found in microglia isolated from aCD3-total CD4+ vs. Iso-total CD4+ and 431 DEGs in aCD3-FoxP3(−)GFP vs. Iso-total CD4+(FIG. 28E). A heatmap signature of the top 1000 DEGs showed a distinct microglial transcriptomic signature of aCD3-total CD4+ compared to Iso-total CD4+ and aCD3-FoxP3(−)GFP groups at 1 month post-CCI(FIG. 28F). Then GOBP pathways was performed, comparing the studied groups and it was found that the aCD3-total CD4+ group was associated with downregulation of several pro-inflammatory pathways involved in innate and adaptive immune responses, immune effector processes and antigen presentation compared to aCD3-FoxP3(−)GFP group. In addition, upregulation in pathways involved in neuron development and morphogenesis and nerve growth factor receptor signaling was observed in aCD3-total CD4+ treated animals (FIG. 28G).

Consistent with the microglial transcriptomic data, RT-qPCR of the ipsilateral hemisphere showed an increase in the expression of several anti-inflammatory cytokines (Il10, Il22, and Il2), and growth factors including Gdnf at 1 month post CCI in the aCD3-total CD4+ group compared to Iso-total CD4+ and aCD3-FoxP3(−)GFP groups (FIG. 28H).

Taken together, these adoptive transfer experiments demonstrate a critical role for CD4+FoxP3+ Tregs in improving behavioral outcomes and attenuating the proinflammatory microglial response following TBI.

### Discussion

Neuroinflammation plays a crucial role in both acute and chronic stages of TBI (Algattas et al. Int J Mol Sci 15, 309-341 (2013)). TBI initiates a complex inflammatory cascade beginning with activation of resident microglia and release of cytokines, followed by peripheral monocyte and lymphocyte recruitment into the CNS which enhances chronic inflammation and contributes to secondary injury (Needham, E. J. et al. J Neuroimmunol 332, 112-125 (2019); Jassam et al. Neuron 95, 1246-1265 (2017)).

It has previously been reported the Treg-dependent immunomodulatory properties (Zhang et al. J Immunol 167, 4245-4253 (2001); Sasaki et al. Circulation 120, 1996-2005 (2009); Ochi et al., Nat Med 12, 627-635 (2006); 408; Ilan, Y. et al. J Cin Immunol 30, 167-177 (2010)) of anti-CD3 mAb in animal models of inflammation and autoimmune diseases (Mayo et al. Brain 139, 1939-1957 (2016); Herold et al. N Engl J Med 346, 1692-1698 (2002); Mathis et al. Pharmacol Res 120, 252-257 (2017); Notley et al. Arthritis Rheum 62, 171-178 (2010)). Nasal anti-CD3 treatment ameliorates chronic inflammatory diseases via the induction of IL-10-dependent CD4+LAP+FoxP3+ Tregs, whereas orally administered anti-CD3 induces TGFβ-1 and its downstream signaling (Wu et al. J Immunol 181, 6038-6050 (2008); Mayo et al. Brain 139, 1939-1957 (2016)). The role of nasal anti-CD3 is unexplored in TBI and other acute brain injury models in which the immune system is reacting to an insult, rather than initiating the insult. Nasal anti-CD3 mAb induced IL-10⁺ FoxP3⁺ Tregs that attenuated chronic microglial inflammation, reduced recruitment of peripheral monocytes and improved the neuropathological and behavioral outcomes following TBI in an IL-10-dependent manner.

Microglia play a critical role in neuroinflammation, and their persistent activation contributes to long-term functional deficits and neurodegeneration (Jassam et al. Neuron 95, 1246-1265 (2017)). A time-dependent change in the microglial transcriptomic phenotype with reduced homeostasis, housekeeping and sensing tissue damage in the early stages following contusional brain injury and with recovery, the transition to a pro-inflammatory state over time have previously been identified (Izzy et al. Front Cell Neurosci 13, 307 (2019)). In the present study, nasal anti-CD3 induced FoxP3⁺ Tregs were shown to enhanced the homeostatic, sensing and housekeeping microglial phenotype at 7 days post-injury, resulting in upregulation of genes such as Tlr1, Cmtm7, Cd33, Cx3cr1, Cd84, and Lag3. Moreover, it was associated with attenuation of chronic microglial proinflammatory transcriptomic phenotype following TBI, resulting in downregulation of proinflammatory genes (Ifitm3, Clec7a, Lgals3, Il6, Casp1, Cd86, Lyz1, Lyz2, Cd40). In addition, at 1 month post-CCI it downregulated MgnD and DAM genes (Tmem119, B2m, Cstb, Cst7, Fth1, Ccl6, Cd9, Cd52, Tyrobp), which are associated with neurodegeneration (Krasemann et al. Immunity 47, 566-581 e569 (2017).

TBI is associated with necrosis and death of neurons. Microglia play a role in recovery by migrating to sites of neuronal death to phagocytose dead or dying cells or debris, participate in synaptic remodeling to minimize neuronal injury and to restore tissue integrity in the injured brain (Hickman et al., Nat Neurosci 21, 1359-1369 (2018).) Nasal anti-CD3 was associated with the upregulation of genes associated with phagocytosis (Cd33 and Cybb) and synaptic pruning and remodeling (Cx3cr1) at 7 days and maintaining myelin homeostasis (Tgfbr1, Tgfbr2, Smad3, Mapk1, Hif1a, Adgrg1, Mertk, Itgav, Atp8a2) at 1-month post-injury. Moreover, it was demonstrated that nasal anti-CD3 treatment increased microglial phagocytic capacity to uptake the apoptotic neurons at 6 days post-injury. It also increased the expression of Bdnf, a key mediator of synaptic plasticity, which increases neuronal TrkB phosphorylation at the site of injury (Houlton et al. Front Neurosci 13, 790 (2019)). In removing cellular debris by phagocytosis early after injury and releasing neurotrophic factors and anti-inflammatory cytokines, microglia contribute to the reduced cell death and improved behavioral and neuropathologic outcomes observed in nasal anti-CD3 group following TBI.

The role of adaptive immunity following TBI is not well understood. Several studies have shown T lymphocyte infiltration into the brain after TBI which plays a role in the neuroinflammatory response following TBI (Xu et al. Cell Prolif 54, e13092 (2021)). Regulatory T cells comprise population of CD4+ T cells that include FoxP3⁺ Tregs and FoxP3⁻ Treg cells, the latter of which includes Th3 and Tr cells (Curotto de Lafaille, et al. Immunity 30, 626-635 (2009)). The therapeutic potential of these Tregs in TBI and their modulatory effects on the CNS innate immune system following injury remains largely unexplored. Deletion of FoxP3⁺ Tregs increased T cell CNS infiltration and expression of inflammatory IFN-γ after TBI. However, the function of the interaction between Tregs and microglia is largely unknown. This study demonstrates that anti-CD3 mAb induced IL-10-producing FoxP3⁺ Tregs that migrate to the CNS to downregulate microglia activation and to improved behavior in an IL-10 dependent manner. Blocking IL-10 receptor in vivo reversed the therapeutic effects of nasal anti-CD3 mAb demonstrating that in TBI the Treg/IL-10 axis is a key immune modulator of the innate immune response and a potential therapeutic target.

A major challenge for the treatment of inflammatory disease is how to induce Tregs in a fashion that is non-toxic and translatable to the clinic. Nasal anti-CD3 treatment is a unique immunotherapeutic approach to stimulate Tregs to downregulate CNS inflammation. Clinically, nasal anti-CD3 mAb could be immediately given to those who have suffered TBI. Nasal administration of Foralumab, a fully humanized anti-CD3 mAb, reduced lung Inflammation and blood inflammatory biomarkers in mild to moderate COVID-19 patients without side effects (Moreira et al. Front Immunol 12, 709861 (2021)). Of note, in animal studies, nasal anti-CD3 mAb was not detected in the brain following nasal administration and did not affect the ability of the lung to clear a bacterial infection (Mayo et al. Brain 139, 1939-1957 (2016)).

In conclusion, this study identifies a novel therapeutic approach that modulates the CNS innate immune response in an IL-10 dependent Treg fashion and is applicable for the treatment of TBI and potentially other types of acute brain injury.

### Materials and Methods

### Experimental Animals

Studies were performed using 8-week-old male C57BL6J mice (000664, Jackson Laboratories), B6.SJL-Ptprca Pepcb/BoyJ B6.CD45.1 mice (002014, Jackson Laboratories) and FoxP3-GFP mice (023800, Jackson Laboratory) including littermates. All mice were housed under specific pathogen free conditions, with food and water ad libitum. All animals were housed in temperature- and humidity-controlled rooms, maintained on a 12-h/12-h light/dark cycle (lights on at 7:00 AM). Mice were euthanized by CO₂ inhalation. The Institutional Animal Care and Use Committee (IACUC) at Harvard Medical School and Brigham and Women's Hospital has all experimental procedures involving animals.

### Treatment

Mice were nasally treated with a daily dose of 1 μg/mouse hamster IgG CD3-specific antibody (clone 145-2C11. BioXCell), or hamster IgG control antibody (BioXCell) dissolved in phosphate-buffered saline (PBS). For some experiments, mice were given 0.5 mg of monoclonal anti-IL-10R blocking antibody (clone 1B1.3A, Bioxcell), by intraperitoneal injection at the onset of TBI for seven straight days and henceforth every third day after the first week until experimental end point.

### Controlled Cortical Impact (CCI)

A CCI model was used as previously described (Bermpohl et al. J Cereb Blood Flow Metab 27, 1806-1818 (2007)). Mice were anesthetized with 4.5% isoflurane (Anaquest) in 70% nitrous oxide and 30% oxygen using a Fluotec 3 vaporizer (Colonial Medical). The mice were placed in a stereotaxic frame and a 5-mm craniotomy was made over the right somatosensory cortex using a drill and a trephine. The bone flap was removed and discarded, and a pneumatic cylinder with a 1.5 or 3-mm flat tip impounder with velocity 6 m/see, depth 1.0 or 1.5 mm, and dwell time of 0.8 s was used to induce CCI(Impact One, Leica Biosystems). The scalp was sutured closed, and the mice were returned to their cages to recover.

### Behavioral Studies

Open Field Testing: The open field (OF) test is used to measure general locomotor activity and anxiety-like behavior of the animals (Kraeuter et al. Methods Mol Biol 1916, 99-103 (2019)). The OF square chambers are made of blue Plexiglas with dimensions of 30×38×40 cm. For each testing session, the animal is allowed to explore the chamber for 15 min. A computer-assisted tracking system and software (Ethovision XT vs. 14, Noldus Information Technology) was used to record the behavior of the animals throughout the testing session. Total distance traveled (cm) and % time spent in the center was measured.

Rotarod: The Rotarod was done as previously described (Mayo et al. Brain 139, 1939-1957 (2016)). Mice were placed on a Rotarod apparatus (Ugo Basile 7650), accelerating from 4-60 RPM in 300 s. Each animal was given three trials and the times when the animal would fall no longer be able to hold on were recorded and averaged for analysis of motor function

Morris Water Maze: The Morris water maze was used to measure spatial learning and memory by training mice to use spatial cues to find a hidden platform to escape water (Vorhees et al. Nat Protoc 1, 848-858 (2006). The Morris apparatus is a circular pool with a diameter of 130 cm and 50 cm deep. During the first day, the platform was visible, and the animals were given three trials to find the platform. During the four-day training period, mice received 3 trials per day learning how to find the hidden platform. Twenty-four hours after the last training day, a probe trial was performed in which the platform was removed, and mice were allowed to swim for up to 60 sec. The amount of time spent by the animal to find the platform and the time spent in the target quadrant for the probe trial were calculated by Noldus EthoVision XT tracking software. Heatmaps were generated by the Ethovision XT software.

### Brain Edema

Brains were removed at 72 h after CCI, bisected into left and right hemispheres, and each hemisphere was weighed (wet weight). Brains were then dried at 60° C. for 48 h, and dry weights were obtained. The percentage of brain water content was expressed as (wet-dry weight)/wet weight×100% as previously described (Wu et al. Cell Death Dis 12, 1064 (2021).

### MRI-Imaging

Imaging was done using 7.0 T Bruker BioSpect® USR. In brief, mice were gently handled and placed in isoflurane anesthesia chamber. Then mice were placed inside the imaging apparatus with their nose in front of tubes releasing 2% of isoflurane. Electrocardiogram (ECG) leads were placed on the animal's paws and a pneumatic pillow sensor will be placed under the abdomen for continuous ECG and respiratory rate monitoring of the anesthetized animal. These waveforms were closely monitored throughout MRI scanning by the MRI operator. The animal was placed on an MRI compatible bed, which will be placed inside the magnet for imaging. The imaging sessions last between 15-60 minutes. Mice were then returned to their cages and were monitored continuously after being returned to their cages prior to returning to a fully alert status. The. Following parameters were obtained to generate the T2 sequence images: Slice Thickness: 0.5 mm, Repetition Time: 3000 ms, Echo Time: 50 ms, Number of Averages: 3, Spacing Between Slices: 0.5 mm, Echo Train Length: 8, Acquisition Matrix: 200×200, Flip Angle: 90, Field of View: 20 mm. Serial Images were viewed and analyzed using the 3D Slicer platform (Fedorov et al. Magn Reson Imaging 30, 1323-1341 (2012)).

### Immunohistochemistry

Animals were anesthetized with CO2 until respiration rate slowed and perfused transcardially with HBSS. Brains were post-fixed in 4% paraformaldehyde for 48 h, then transferred to a 15% sucrose solution for 24 h, then finally transferred to a 30% sucrose solution for 24 h. Brains were then flash frozen in Tissue-Tek Oct (Sakura, Compound 4583) and stored at −80C until the time of sectioning. Brains were subsequently sectioned at −20C using cryostat at the Bregma position for each targeted brain. Sections were cut at 0.2 mm in a 4-fold series interval. 5 total sections were placed on Colorfrost Plus™ treated adhesion slides (Thermo Fisher Scientific, Waltham, MA, USA) and stored at −20C until the time of staining. For immunofluorescence sections were blocked in in a 10% normal horse serum solution, containing 0.1% Triton X-100, 1% glycine, and 2% bovine serum albumin. Slides were incubated over night at for 4C with Anti Iba1 (rabbit, 1:1000, Wako). The Following day sections were washed and incubated with an AlexaFluor 647 goat anti-rabbit IgG (1:1000, Abcam, ab150075) for 1 hour at room temperature. Sections were also stained with haematoxylin and eosin (HE; Abcam, ab245880), and TUNEL (TUNEL Assay Kit—BrdU-Red, Abcam, ab66110) according to their corresponding kit protocols. Iba-1 and TUNEL stained slides were co-stained with DAPI mounting media (Vector Laboratories, UX-93952-24). 5 animals per group were used for each stain. Images were taken using a Leica DMi8 Widefield Microscope on the 20× objective.

### Image Analysis

Analysis of percent Iba-1, and number of TUNEL positive cells per surface area was performed on 5 photomicrographs per animal (n=4 or 5). The sections analyzed were taken between 300 and 1500 micrometers laterally from the coronal plane. Each scanned photomicrograph was used to produce images of the area of contusion. All the images were analyzed using ImageJ software (National Institute of Health, https://imagej.nih.gov/ij/). Images were split by color channel, and the channel of interest was threshold using the Yen setting and the percent area and number of positive cells were quantified as previously described (Izzy et al. Int J Mol Sci 22 (2021)).

### Flow Cytometry Microglial Sorting

For microglial cell sorting, mice were anesthetized with CO2 until respiration rate slowed and then transcardially perfused with 50 mL hanks balanced salt solution (HBSS) containing heparin (1:1000). Following perfusion, the ipsilateral hemisphere homogenised using a dounce glass tissue homogeniser. Cells were separated through Percoll (GE Healthcare Life Sciences) 30% gradient centrifugation. Cells were isolated from the Percoll layer and stained on ice for 30 min with combinations of PE/Cy7 rat anti-mouse CD11b (Biolegend, #101216, 1:100), APC/Cy7 rat anti-mouse CD45 (Biolegend, #103116, 1:100), FITC rat anti-mouse Ly6C (Biolegend, #128006, 1: 200) and APC rat anti-mouse 4D4 (Krasemann et al. Immunity 47, 566-581 e569 (2017)). (marking resident microglia; 1:1000) in blocking buffer containing 0.2% bovine serum albumin (BSA, Sigma-Aldrich) in HBSS. Cell sorting was performed using FACSAriaIII cell sorter (Becton Dickson). Microglial cells were identified as CD45+CD11b+Ly6C-4D4+ and Dead cells were also excluded based on 7-AAD (BD Bioscience) staining. Cells were sorted directly in 1.5 mL Eppendorf tubes and stored at −80° C.

### Flow Cytometry Intracellular Staining

Intracellular cytokine staining and cell isolation was done as previously described (Rezende et al. Nat Commun 9, 3151 (2018)). The ipsilateral brain hemispheres were isolated were isolated using the neuronal tissue dissociation kit (P) (Miltenyi Biotec #130-092-628) according to the manufacturer's specification. Following the enzyme dissociation, the cells were separated using Percoll (GE Healthcare Life Sciences) as described above. Cells isolated from the brain were only incubated for 2 hours instead of the 4 hours for both the splenic and cLN cells. Flow-cytometric acquisition was performed on a Fortessa or Symphony (BD Biosciences) by using DIVA software (BD Biosciences) and data were analyzed with FlowJo software versions 9.9 or 10.1 (TreeStar Inc.). Intracellular staining antibodies used Zombie Aqua Fixable Viability Kit (Biolegend, #423102, 1:1000) or Zombie UV (Biolegend, #423108, 1:1000) was used to exclude dead cells. The staining antibodies used are AF700 anti-CD45 (Biolegend, #103128, 1:200), BV785 anti-CD11b (BD Biosciences, #740861, 1:200), BV605 anti-CD3ε (Biolegend, #100351, 1:100), PE/Cyanine 7 anti-TCR-beta (Biolegend #109222, 1:100, BUV661 anti-CD45 (BD Biosciences, #565079, 1:200), PE anti-CD4 (BD Biosciences, #553730, 1:100), FITC anti-FoxP3 (eBioscience, #11-5773-82, 1:100), PE anti-LAP (Biolegend, #141404, 1:100), PE/Dazzle 594 anti-IL10 (Biolegend, #505034, 1:100), BV570 anti-CD19 (Biolegend, #127639, 1:100), BV605 anti-Ly6G (Biolegend, #127639, 1:100), APC anti-FCRLS⁶⁴ (1:1000) provided by Dr. Butovsky, BUV395 anti-NK1.1 (BD Biosciences, #564144, 1:100), and AF700 anti-Ly6C (Biolegend, #128024, 1:200).

### Quantitative Polymerase Chain Reaction

RNA was extracted with RNeasy® columns (Qiagen), cDNA was prepared and used for quantitative PCR (Applied Biosystems™, 437466) and the results were normalized to Gapdh (Mm99999915_g1). AppliedBiosystems, IL10 (Mm01288386_m1), IL6 (Mm00446190_m1), Tnf (Mm00443258_m1), IL-1b (Mm00434228_m1), IL-2 (Mm00434256_m1), IL-23a (Mm00518984_m1), IL-18 (Mm00434226_m1), INFg (Mm01168134_m1), Gapdh (Mm00484668_m1), IL-3 (Mm00439631_m1), IL-27 (Mm00461162_m1), Tgfa (Mm00446232_m1), IL18 (Mm00434226_m1), IL12a (Mm00434169_m1), Bdnf (Mm04230607_s1), Gdnf (Mm00599849_m1), CCL5 (Mm01302427_m1), Csf1 (Mm00432686_m1), Lgals3 (Mm00802901_m1), Ifitm3 (Mm00847057_s1), Stat1 (Mm01257286_m1), Axl (Mm01169744_m1), CD14 (Mm01158466_g1), Mrc1 (CD206)(Mn01329359_m1), IL4 (Mn00445259_m1), Tgfb1 (Mm01178820_m1), IL-17a (Mn00439618_m1), IL-21 (Mm00517640_m1). 2−ΔΔCt method was used to calculate relative expression of each gene.

### Isolation of Primary Neurons

Primary neuron isolation was done as previously described (Krasemann et al. Immunity 47, 566-581 e569 (2017)). In short, primary neurons were prepared from embryos at age E18. Cell density was determined using a hemocytometer and cells were seeded. DMEM with 10% FBS was used for initial plating, and the medium was changed to Neurobasal supplemented with 1×B27 (Invitrogen) 3 h later. Media was changed every 3 days.

### Induction of Apoptosis and Labeling of Neurons

Apoptosis and labeling of neurons was done as previously described (Krasemann et al. Immunity 47, 566-581 e569 (2017)). Neurons were irradiated with UV light (302 nm) with intensity of 6×15 W for 15 min. The apoptotic neurons were labelled with labeling dye (Alexa488 5-SDP Ester or Alexa405 NHS Ester, Life Technologies/Thermo Fisher Scientific). Neurons were resuspended at a density of 260,000 cells per 4 uL for stereotactic injections.

### Stereotactic Injections

Mice were anesthetized by intraperitoneal injection of Ketamine (100 mg/kg). Apoptotic neurons or Sterile DPBS were injected in the lesion of TBI mice at two depths of 1 mm and 2 mm. 2 uL were injected at each depth using stereotaxic equipment (Harvard Apparatus). After recovery from surgery, animals were returned to their cages. Post-surgery (16 h), mice were euthanized by CO₂ inhalation and brains were processed for flow cytometry analysis of phagocytic microglia.

### Adoptive Transfer

To test the in vivo regulatory function of the nasally induced T cells, freshly isolated whole splenic CD4+ or CD4+ T cells depleted of FoxP3+ cells from anti-CD3 or isotype control treated TBI mice (CD45.2) during the acute phase of TBI (Day 7) were transferred to a new cohort of TBI (CD45.1) mice at the onset of TBI, day 14, and day 30. Each recipient received 2.5×10⁶ T cells intravenously. Splenocytes were purified and enriched using CD4 cell isolation microbead kit (Militenyi Biotech, #130-104-454) on a magnetic MACS separator prior to sorting. Cell sorting was performed using FACSAriaIII cell sorter (Becton Dickson) and APC anti-mouse CD4 antibody (GK1.5, Biolegend) and 7-AAD (BD Bioscience) was used to identify the live CD4+ population and the FITC channel was used to exclude the FoxP3+ population in the FoxP3+ depleted CD4+ population.

### In Vitro Cell Culture

Sorted 4D4+ microglia 24 hours post TBI were cultured as previously described (Xie et al. Eur J Immunol 45, 180-191 (2015)). at a number of 200,000 cells in a 24 well plate (Kemtec™ 4422A). The microglia culture media composed of 10% fetal bovine serum (FBS; Gibco, #10438026), 100 U/mL penicillin-streptomycin mixture (Lonza, #DE17-602E), supplemented in Dulbecco's Modified Eagle Medium (DMEM)/F-12 Glutamax media (Gibco, #10565018). into a Total CD4+ Tregs from 7 days anti-CD3 and Isotype control treated TBI mice were sorted into a lymphocyte culture media composed of 10% fetal bovine serum (FBS; Gibco, #10438026), 100 U/mL penicillin-streptomycin mixture (Lonza, #DE17-602E), 55 μM 2-mercaptoethanol (Gibco, #21985023), 1% sodium pyruvate (Lonza, #BE13-115E) and 1% HEPES (Lonza, #BE17-737E) supplemented in Roswell Park Memorial Institute (RPMI) 1640 media (Gibco, #11875119) and placed on the top of the hanging cell culture 0.4 μm insert (Millicell, PTHT24H48) at 800,000 cells per insert and placed on top of the cultured microglia and the assay was left for 72 hours in. a CO2 Cell culture Incubator (InCusafe). After 72 hours the microglia were lysed with Buffer RLT and RNA was extracted with RNeasy® columns (Qiagen) and qPCR was done.

### Bioinformatics:

Microglia Bulk RNA-Sequencing: Bulk RNA sequencing was performed as previously described (Butovsky et al. Nat Neurosci 17, 131-143 (2014)). Briefly, 2,000 isolated microglia CD45+CD11b+Ly6C-4D4+ were lysed in 5 ul TCL buffer+1% β-mercaptoethanol. Smart-Seq2 libraries were prepared and sequenced by the Broad Genomic Platform. cDNA libraries were generated from sorted cells using the Smart-seq2 protocol 5. RNA sequencing was performed using Illumina NextSeq500 using a High Output v2 kit to generate 2×38 bp reads. The processing of the bulk RNA-seq data was based on an established computational pipeline (Pertea et al. Nat Protoc 11, 1650-1667 (2016)). Sequencing data were demultiplexed and provided by the Broad Institute in FASTQ format. FastQC was used to assess sequencing quality control. Trimmomatic was used for adaptor trimming of reads. Reads were then aligned to the ‘mm10’ reference genome using HISAT. The generated SAM files were then converted into BAM files using SAMtools. StringTie was used for transcript assembly and quantification. Transcript abundances were then imported into R Studio (version 4.1.2) and converted to gene-level estimated counts using the ‘tximport’ package (version 1.22.0) from Bioconductor. Genes that achieved less than 10 counts summed across all samples were considered very low expressed genes and thus filtered out. Sample read counts were normalized using the variance stabilizing transformation method (VST) from the DESeq2 (version 1.34.0) built-in VST function. These normalized sample read counts were used to plot heatmaps using pheatmap (version 1.0.12) and ComplexHeatmap (version 2.13.1) and bar-plots using ggpubr (version 0.4.0) and ggplot2 (version 3.3.6). Principal component analysis (PCA) plots were generated by utilization of the DESeq2 built-in PCA function using the default settings.

There were three different cohorts which underwent separate RNA-sequencing: nasal anti-CD3 cohort, nasal anti-CD3/anti-IL10R cohort, and the adoptive transfer cohort. For the nasal anti-CD3 cohort, there were two timepoints: 7 days and 1-month post-TBI. Samples were divided into three different groups for each timepoint separately: Sham-Iso, TBI-Iso, and TBI-aCD3. For the nasal anti-CD3/anti-IL10R cohort, samples were divided into three different groups at 1-month post-TBI: Sham-Iso, TBI-Iso, and TBI-aCD3+aIL10R. For the adoptive transfer cohort, samples were divided into three different groups at 1-month post-TBI: Iso-total CD4+, aCD3-total CD4+, and aCD3-FoxP3(−)GFP. To directly compare the differences in gene expression between TBI-aCD3 and TBI-aCD3+aIL10R with TBI-Iso and Sham-Iso, the nasal anti-CD3/anti-IL10R cohort was integrated with the 1-month post-TBI group of the anti-CD3 cohort and batch effects were corrected using ComBat-seq⁹¹ through the sva package (version 3.42.0).

Differential Gene Expression and Pathway Analysis: Differential gene expression analysis was carried out with DESeq2. Genes identified using DESeq2 that featured a P value<0.05 (Benjamini-Hochberg method) were considered significant differentially expressed genes (DEGs). Comparisons of gene expression across three or more sample groups were done using the reduced Likelihood Ratio Test (LRT) method. For pair-wise comparisons of gene expression between two different sample groups and for pathway analysis, the Wald Test was used with standard parameters and log 2 fold-changes were subsequently shrunken using DESeq2. Pair-wise comparisons of differentially expressed genes were visualized using DiVenn. All gene set and pathway analyses were performed through the GAGE package (version 2.44.0). Statistical significance for all pathway analyses and tests was defined as P value<0.05.

Statistical analysis: Statistical analysis was performed using GraphPad Prism 9 software. Data are presented as mean±s.e.m and Student's t tests (unpaired) or One-way and Two-way ANOVA multiple comparison tests with Tukey's multiple comparisons was used to assess statistical significance between the groups were used to assess statistical significance. All n and P values and statistical tests are indicated in figure legends.

### REFERENCES

1. Wang M, Gao M, Miller K D, Zheng Q H. Synthesis of [(1)(1)C]PBR06 and [(1)(8)F]PBR06 as agents for positron emission tomographic (PET) imaging of the translocator protein (TSPO). Steroids 2011; 76:1331-40.
2. Lartey F M, Ahn G O, Shen B, et al. PET imaging of stroke-induced neuroinflammation in mice using [18F]PBR06. Mol Imaging Biol 2014; 16:109-17.
3. James M L, Belichenko N P, Nguyen T V, et al. PET imaging of translocator protein (18 kDa) in a mouse model of Alzheimer's disease using N-(2,5-dimethoxybenzyl)-2-18F-fluoro-N-(2-phenoxyphenyl)acetamide. J Nucl Med 2015; 56:311-6.
4. Singhal T, O'Connor K, Dubey S, et al. 18F-PBR06 Versus 11C-PBR28 PET for Assessing White Matter Translocator Protein Binding in Multiple Sclerosis. Clin Nucl Med 2018; 43:e289-e95.
5. Singhal T, O'Connor K, Dubey S, et al. Gray matter microglial activation in relapsing vs progressive MS: A [F-18]PBR06-PET study. Neurol Neuroimmunol Neuroinflamm 2019; 6:e587.
6. Singhal T, Cicero S, Pan H, et al. Regional microglial activation in the substantia nigra is linked with fatigue in MS. Neurol Neuroimmunol Neuroinflamm 2020; 7.
7. Owen D R, Yeo A J, Gunn R N, et al. An 18-kDa translocator protein (TSPO) polymorphism explains differences in binding affinity of the PET radioligand PBR28. J Cereb Blood Flow Metab 2012; 32:1-5.
8. Dickstein L P, Zoghbi S S, Fujimura Y, et al. Comparison of 18F- and 11C-labeled aryloxyanilide analogs to measure translocator protein in human brain using positron emission tomography. Eur J Nucl Med Mol Imaging 2011; 38:352-7.
9. Nair A, Veronese M, Xu X, et al. Test-retest analysis of a non-invasive method of quantifying [(11)C]-PBR28 binding in Alzheimer's disease. EJNMMI Res 2016; 6:72.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating or alleviating a sign or symptom of amyotrophic lateral sclerosis (ALS) in a subject, the method comprising administering to the subject by intra-nasal administration a daily dose between 25-175 μg of an anti-CD3 antibody for at least 3 months.

2. The method of claim 1, wherein the anti-CD3 antibody is a monoclonal antibody.

3. The method of claim 1, wherein the anti-CD3 antibody is a fully human or humanized.

4. The method of claim 1, wherein the anti-CD3 antibody comprises a heavy chain complementarity determining region 1 (CDRH1) comprising the amino acid sequence GYGMH (SEQ ID NO: 1), a heavy chain complementarity determining region 2 (CDRH2) comprising the amino acid sequence VIWYDGSKKYYVDSVKG (SEQ ID NO: 3), a heavy chain complementarity determining region 3 (CDRH3) comprising the amino acid sequence QMGYWHFDL (SEQ ID NO: 4), a light chain complementarity determining region 1 (CDRL1) comprising the amino acid sequence RASQSVSSYLA (SEQ ID NO: 5), a light chain complementarity determining region 2 (CDRL2) comprising the amino acid sequence DASNRAT (SEQ ID NO: 6), and a light chain complementarity determining region 3 (CDRL3) comprising the amino acid sequence QQRSNWPPLT (SEQ ID NO: 7)

5. The method of claim 1, wherein the anti-CD3 antibody comprises a variable heavy chain amino acid sequence comprising the amino acid sequence of SEQ ID NO: 8 and a variable light chain amino acid sequence comprising the amino acid sequence of SEQ ID NO: 9.

6. The method of claim 1, wherein the anti-CD3 antibody comprises a heavy chain amino acid sequence comprising the amino acid sequence of SEQ ID NO: 10 and a light chain amino acid sequence comprising the amino acid sequence of SEQ ID NO: 11.

7. The method of claim 1, wherein the daily dose is between 25-100 μg.

8. The method of claim 7, wherein the daily dose is about 50 μg or about 100 μg.

9. The method of claim 1, wherein the intra-nasal administration is by nasal spray, wherein each administration delivers 25-200 μL.

10. The method of claim 1, wherein the daily dose is split equally between each nostril.

11. The method of claim 1, wherein the daily dose is administered in a single nostril.

12. The method of claim 1, wherein the daily dose is administered three times a week.

13. The method of claim 1, wherein the daily dose is administered to the subject in at least one cycle, wherein the cycle is once daily three times a week for two weeks.

14. The method of claim 13, wherein the cycle is followed by a drug holiday.

15. The method of claim 14, wherein the drug holiday is a week.

16. The method of claim 1, wherein the subject is administered the anti-CD3 antibody for at least 6 months, at least 9 months, at least 12 months, at least 18 months, at least 2 years, at least 3 years, or at least 5 years.

17. The method of claim 1, wherein the daily dose is sufficient to induce a reduction in microglial activation of at least 20%.

18. The method of claim 17, wherein microglial activation is reduced in cerebral cortex, thalamus, white matter, cerebellum, or whole brain.
